(19) United States
(12) Patent Application Publication
(10) Pub. No.: US 2019/0365332 A1
(43) Pub. Date: Dec. 5, 2019

(54) DETERMINING WELLNESS USING ACTIVITY DATA

(71) Applicant: Gero LLC, Moscow (RU)

(72) Inventors: Petr Olegovich FEDICHEV, Dolgopmdniy (RU); Sergei Alexandrovich FILONOV, Moscow (RU); Maksim Nikolaevich KHOLIN, Moscow (RU); Timofey Vladimirovich PYRKOV, Moscow (RU); Glib Igorevich IVASHKEVYCH, Moscow (RU); Stanislav Alexandrovich LEONTENKO, Moscow (RU); Evgeny Gennadievich GETMANTSEV, Moscow (RU); Boris Arturovich ZHUROV, Zelenograd (RU)

(21) Appl. No.: 16/448,556

(22) Filed: Jun. 21, 2019

Related U.S. Application Data
(63) Continuation of application No. PCT/RU2017/050126, filed on Dec. 12, 2017.

(30) Foreign Application Priority Data
Dec. 21, 2016 (RU) RU2016150479

(51) Int. Cl. A61B 5/00; A61B 5/11; G16H 50/30

(52) U.S. Cl. CPC A61B 5/7282; A61B 5/1118; A61B 5/6898; G16H 50/30; A61B 5/681; A61B 5/1123

(57) ABSTRACT

Methods and apparatus comprise a model to accurately assess and track changes in physical activity and locomotor patterns measured by an activity sensor such as accelerometer or step counter of mobile and wearable devices to evaluate the age, hazard rate or hazard ratio, frailty, obesity and type 2 diabetes status. The model is capable of detecting age-related and age-independent hazard rate or hazard ratio and other related parameters such as age, biological age, frailty, obesity and type 2 diabetes status that are detectable in activity sensor data acquired from freely moving subjects engaged in routine activities. The disclosed methods and apparatus have sufficient accuracy for practical implementation in personal and corporate wellness with readily available mobile devices such as personal smartphones and wristbands.

### CROSS-REFERENCE TO RELATED APPLICATIONS

This application is a continuation of PCT Application No. PCT/RU2017/50126 filed on Dec. 12, 2017, which claims priority to Russian Patent Application No. RU2016150479 filed on Dec. 21, 2016, the entireties of which are incorporated herein by reference.

### BACKGROUND

Prior methods and apparatus to determine the wellness of subjects can be less than ideal in at least some respects. The prior methods and apparatus can be overly complex, and may not adequately identify people who are not living a healthy lifestyle or who may be at risk. Also, many of the prior approaches do not assess freely moving physical activity of subjects and in some instances can be used only in laboratory or controlled experimental conditions and/or require a subject to perform sets of specially designed medical or physical activity examinations, which may be less than ideal solutions to health assessment.

Many people are exposed to environmental factors of modern society, leading to elevated stress levels, unhealthy lifestyles often characterized by lack of exercise, sleep disturbance, consuming unhealthy food. These present the risk factors to personal wellness and successful aging. Unfortunately, at least some of these risk factors are not timely recognized in many instances, resulting in health conditions in many people that may result in disease and may require medical interventions. Such medical interventions can place a significant burden on social healthcare systems. An example of a condition which places a significant and increasing burden on the health care system of some countries is type 2 diabetes. Early detection of lifestyle risk factors leading to premature aging and type 2 diabetes would be helpful for implementing lifestyle interventions including physical exercise, maintaining normal body weight, and healthy diet before the onset of pathological conditions.

Although screening and monitoring to detect health risk factors have been proposed, the prior methods and apparatus are less than ideally suited to detect at risk individuals to address the health of a population on a large scale. For example, although healthcare organizations can conduct tests, the complexity and amount of time required for such tests can result in fewer people being examined and fewer conditions being detected in a timely manner. Also, tests conducted in a clinic do not present a complete picture of the activity and lifestyle of a person on a daily basis and may be unable to examine a wide population in a reasonable time range. While physical activity measurements have become affordable and cost-effective with advances in technology of low power and compact sensors, the prior utilization of such data to detect lifestyles and at risk individuals has been less than ideal. For example, although publicly available databases such as the National Health and Nutrition Examination Survey (NHANES) and UK biobank provide accelerometer data and health related conditions on people, the utilization of such data to determine the health status of an individual outside the study group has been unable to achieve an acceptable accuracy level without using additional questionnaire information in at least some instances.

Although prior studies have attempted to assess risk factors of subjects for diabetes and mortality based on accelerometer data, at least some of these studies have not utilized aspects of the data which could be helpful in assessing age, diabetes and hazard rate or hazard ratio, for example, in freely moving conditions. Data taken outside of the clinical setting can result in data which has imperfections, is sampled intermittently, and may in some instances require a specialized approach to extract quantitative characteristics that are robust to noise. There is a need for more accurate health risk assessment under conditions of freely moving activity where the subject is allowed to engage in normal and random activity and the physical activity levels are not regulated by the experimental conditions.

In light of the above, it would be advantageous to have more accurate methods and apparatus for accurate assessment of health and wellness parameters capable of being used on a population without the need to attend specialized health or research centers and without interfering with one's routine daily life. Ideally, such assessment methods and apparatus would be suitable for combination with compact sensors embedded in wearable and carried devices that are capable of measuring acceleration, motion, and/or heart rate, to assess the health and wellness of an individual and populations

### SUMMARY

The present disclosure relates generally to assessment and tracking of individual wellness and health risk factors with models responsive to measurements of physical activity and locomotor patterns. The present methods and apparatus can be used to assess and track changes in physical activity and locomotor patterns measured by an activity sensor such as accelerometer or step counter of mobile and wearable devices to evaluate the age, hazard rate or hazard ratio, frailty, obesity and type 2 diabetes status risk factors. The present disclosure in many instances relates to evaluation of hazard rate or hazard ratio in the field of survival analysis, or in the field of analysis of disease onset and remission, and to evaluation of age, frailty, obesity, type 2 diabetes status as the important factors of hazard rate and hazard ratio. Although specific reference is made to human subjects, the methods and apparatus disclosed herein will have application in many fields, such as laboratory animals, farm animals and companion animals.

The presently disclosed methods and apparatus provide improved assessment and monitoring of individuals and populations with models having improved accuracy to detect health risks that may lead to pathological conditions. Work in relation to the present disclosure indicates that age, hazard rate or hazard ratio, frailty, obesity and type 2 diabetes status risk factors present biomarkers that are detectable in activity sensor data acquired from freely moving subjects engaged in routine activities. The disclosed methods and apparatus have sufficient accuracy for practical implementation with readily available mobile devices such as personal smartphones and wristbands. The methods and apparatus disclosed herein can be used without any special user skill and do not interfere with routine daily activities. Personalized recommendations can be provided for individuals and population groups related to wellness, health, nutrition, fitness and other aspects of personal lifestyle. The methods and apparatus disclosed herein can be used to assess individual health risk factors, as a screening tool for early detection of various health problems to prevent chronic diseases, and to reduce the likelihood of recurrence. The present methods and apparatus can also be used for stratification of groups based on risks, as well as for many other practical applications related to population health, and can be used by authorized healthcare, insurance agencies and employers.

In some embodiments, data is obtained from an activity sensor such as an accelerometer externally coupled to the subject when the subject is free to move and conduct daily activities, and a feature extracted from the data as a quantitative characteristic of physical activity and locomotor patterns. The data can be arranged to provide low resolution signals for feature extraction in accordance with a model, and the features can be derived from frequencies below about 1 Hz. Alternatively or in combination, the feature can be extracted from high resolution data corresponding to frequencies above about 1 Hz. The feature can also be derived from transitions among activity levels of the user, and low resolution data or high resolution data from transitions among activity levels can be used to extract the feature. The data can be arranged in many ways to provide low resolution signals for feature extraction, for example by arranging a series of data to provide low resolution series of sensor measurements to extract the features. The low resolution data may comprise pedometer measurements arranged in sequence for feature extraction, for example. High resolution sensor data such as data sampled directly from an accelerometer can be processed to provide low resolution sensor data for feature extraction. The features extracted from high resolution data of the activity sensor can provide improved accuracy of the evaluated wellness parameter such as age, hazard rate or hazard ratio, frailty, obesity and type 2 diabetes status risk factors.

In some embodiments, the extracted features used with the model comprise quantitative characteristics related to characteristics of the sensor data, such as power spectral density, autocorrelation or probability distribution of activity states, autocorrelation or probability distribution of occupancy of density states, or autocorrelation or probability distribution of transitions between activity states. The activity states may comprise different activity levels or different patterns in the sensor data. Data obtained from the sensor in freely moving conditions may be subject to noise and, in many instances, an predetermined amount of data may be accumulated before the evaluation is performed. The presently disclosed methods and apparatus may perform feature extraction in a manner that is robust to noise generated in freely moving conditions, thus enabling its use by a user without any special skills or training for collection of sensor data.

In a first aspect, a method to evaluate a wellness parameter of a subject in response to freely moving physical activity of the subject comprises receiving a plurality of measurements of freely moving physical activity of the subject obtained by a sensor coupled to the subject. A feature from the plurality of measurements is extracted. The wellness parameter of the subject is evaluated with a model in response to the feature. In some instances, the wellness parameter is selected from the group consisting of an age, a hazard rate, a hazard ratio, a type 2 diabetes status and a body mass index.

In another aspect, a method to evaluate a wellness parameter or a derived parameter of a subject in response to freely moving physical activity of the subject comprises evaluating the wellness parameter or the derived parameter with a model in response to the plurality of features, extracted from the plurality of measurements of freely moving physical activity of the subject received by a sensor externally coupled to the subject. The wellness parameter is selected from the group consisting of an age, a hazard rate, a hazard ratio, a type 2 diabetes status and a body mass index. In some instances, the derived parameter is evaluated in response to the evaluated wellness parameter. Optionally, the derived parameter is evaluated in response to a plurality of evaluated wellness parameters.

In another aspect, a method to output or use a wellness parameter or a derived parameter of a subject comprises outputting or using the wellness parameter or the derived parameter evaluated with a model in response to a plurality of features, extracted from a plurality of measurements of freely moving physical activity of the subject obtained by a sensor externally coupled to the subject. The wellness parameter is selected from the group consisting of an age, a hazard rate, a hazard ratio, a type 2 diabetes status and a body mass index. In some instances, the derived parameter is evaluated in response to the evaluated wellness parameter. Optionally, the derived parameter is evaluated in response to a plurality of evaluated wellness parameters.

In another aspect, a tangible medium is configured with instructions that when executed cause a processor to perform the method.

In another aspect, an apparatus to evaluate a wellness parameter of a subject in response to freely moving physical activity of the subject comprises a processor comprising the tangible medium.

In some embodiments, the apparatus further comprises an activity sensor.

In some embodiments, the apparatus further comprises a computer chip. The computer chip comprises the processor. The computer chip is coupled to the activity sensor to receive the plurality of measurements from the activity sensor. The processor is configured with instructions to extract a plurality of features from the plurality of measurements and output the wellness parameter of the subject. The computer chip is configured to be carried by the subject and optionally comprises a length, a width and a height of no more than 5 mm, 5 mm and 3 mm, respectively.

In another aspect, a tangible medium to evaluate a wellness parameter of a subject in response to freely moving physical activity of the subject is configured with instructions that when executed cause a processor to receive a plurality of measurements of freely moving physical activity of the subject from a sensor coupled to the subject, and output the wellness parameter in response to the plurality of measurements with an output device, the output device selected from the group consisting of a display, an audio output, a haptic output and a brain computer interface. The wellness parameter of the subject is selected from the group consisting of an age, a hazard rate, a hazard ratio, a body mass index and a diabetes type 2 status.

In another aspect, a method to evaluate a wellness parameter of a subject in response to freely moving physical activity of the subject comprises receiving a plurality of measurements of freely moving physical activity of the subject obtained by a sensor coupled to the subject, and outputting the wellness parameter. The wellness parameter is selected from the group consisting of an age, a hazard rate, a hazard ratio, a type 2 diabetes status and a body mass index. Optionally, the wellness parameter has been evaluated in response to a feature extracted from the plurality of measurements.

In some embodiments, a derived parameter is evaluated with the model in response to a wellness parameters, the wellness parameter selected from the group consisting of an age, a hazard rate, a hazard ratio, a type 2 diabetes status and a body mass index. Optionally, the derived parameter is evaluated in response to a plurality of wellness parameters selected from the group consisting of the age, the hazard rate, the hazard ratio, the type 2 diabetes status and the body mass index.

In some embodiments, the derived parameter is output. The output wellness parameter is used to evaluate the derived parameter and optionally the plurality of wellness parameters used to evaluate the derived parameter.

In some embodiments, the feature is derived from frequencies of activity sensor data of no more than about 1 Hz. Optionally, the feature is derived from frequencies above about 1 Hz.

In some embodiments, the feature is derived from activity sensor data having frequencies selected from the group consisting of no more than about 0.1 Hz, no more than about 0.01 Hz, no more than 0.001 Hz, no more than about 0.0001 Hz, no more than about 0.0002, no more than about 0.000011 Hz and no more than about 0.00001 Hz.

In some embodiments, the feature is derived from activity sensor data comprising an average power spectral density selected from the group consisting of no more than about 0.1 Hz, no more than about 0.01 Hz, no more than 0.001 Hz, no more than about 0.0001 Hz, no more than about 0.0002, no more than about 0.000011 Hz, and no more than about 0.00001 Hz.

In some embodiments, the feature comprises a plurality of features derived from frequencies from activity sensor data of no more than about 100 Hz.

In some embodiments, the feature comprises a plurality of features derived from frequencies from activity sensor data of no more than about 1 Hz.

In some embodiments, the plurality of features is derived from transitions among activity levels having frequencies selected from the group consisting of no more than about 0.1 Hz, no more than about 0.01 Hz, no more than 0.001 Hz, no more than about 0.0001 Hz, no more than about 0.0002, no more than about 0.000011 Hz, and no more than about 0.00001 Hz.

In some embodiments, the feature is derived from activity sensor data having frequencies within a range selected from the group consisting of any two of the following values: 0.1 Hz, 0.01 Hz, 0.001 Hz, 0.0001 Hz, 0.0002 Hz, 0.000011 Hz, 0.00001 and 0.000001 Hz.

In some embodiments, the plurality of measurements comprises a series of data. Data of the series of data is separated by a time within a range selected from the group consisting of one millisecond to one second, one millisecond to one minute, one millisecond to one hour, one millisecond to one day, five seconds to one minute, five seconds to one hour five seconds to one day, one minute to one hour, and one minute to one day.

In some embodiments, the plurality of measurements comprises of a low-resolution series of measurements.

In some embodiments, the plurality of measurements comprises a high-resolution series of measurements.

In some embodiments, the plurality of measurements comprises a series of measurements selected from the group consisting of a low-resolution series of measurements and a high-resolution series of measurements. In some instances, the low-resolution series of measurements represents a low resolution time evolution profile of physical activity levels of the subject with a time resolution longer than 1 s and optionally with the time resolution within a range from 5 seconds to 1 hour. In some instance, the high-resolution series of measurements represents a fast time evolution profile of a physical quantity related to motion of the subject with the time resolution shorter than 1 s and optionally with a time resolution shorter than 250 ms.

In some embodiments, a shortest time resolution of the plurality of measurements is 0.01 s. In some instances, the shortest time resolution of the plurality of measurements is 0.05 s.

In some embodiments, each high-resolution series of measurements has time resolution selected from the group consisting of shorter than 100 ms, shorter than 50 ms and shorter than 20 ms.

In some embodiments, the plurality of measurements comprises a plurality of measurements that meet requirements of preprocessing filtering. In some instances, the requirements correspond to the total accumulated amount of a low-resolution series of measurements. In some instances, the requirements correspond to a total accumulated daily amount of measurements sampled on one day from the subject, the total accumulated daily amount within a range from 8 to 16 hours after the subject woke up.

In some embodiments, a total accumulated amount of a set of low-resolution series of measurements corresponds to at least about 12 hours of activity, at least about 24 hours, at least about 48 hours, and at least about 72 hours. In some instances, an interval between a first measurement and a last measurement of the total accumulated amount is selected from the group consisting of at least about 1 day, at least about 3 days and at least about 1 week. In some instances, the total accumulated amount corresponds to freely moving physical activity over a plurality of days.

In some embodiments, a set of low-resolution series of measurements comprises measurements sampled within a range from 8 to 16 hours after the subject woke up and a total accumulated amount of low-resolution series of measurements sampled within the range from 8 to 16 hours after the subject woke up corresponds to at least 4 hours of freely moving physical activity. In some instances, the total accumulated amount within the range corresponds to amounts measured over a plurality of days. In some instances, the total accumulated amount is selected from the group consisting of at least 8 hours and at least 24 hours.

In some embodiments, a set of high-resolution series measurements corresponds to at least about 3 hours, at least about 6 hours, at least about 12 hours, at least about 24 hours of freely moving physical activity of the subject. In some instances, an interval between a first measurement and a last measurement of the set of high resolution series measurements is selected from the group consisting of at least about 1 day, at least about 3 days, and at least about 1 week. In some instances, the total accumulated amount corresponds to freely moving physical activity over a plurality of days.

In some embodiments, a set of high-resolution series of measurements comprises measurements sampled between 8 and 16 hours after the subject woke up and total accumulated amount of high-resolution series of measurements sampled between 8 and 16 hours after the subject woke up corresponds to at least 3 hours of freely moving physical activity. In some instances, the total accumulated amount within the range corresponds to at least 6 hours of freely moving physical activity. In some instances, the total accumulated amount within the range corresponds to at least 12 hours of freely moving physical activity. In some instances, the total accumulated amount within the range corresponds to amounts measured over a plurality of days.

In some embodiments, the apparatus, tangible medium, computer chip, or method further comprises a preprocessing procedure to filter out series of measurements which span too short time range, or have inappropriate time resolution, or correspond to activity level period of inappropriate length, or otherwise are not appropriate for the evaluating of the wellness parameter.

In some embodiments, the apparatus, tangible medium, computer chip, or method further comprises preprocessing the plurality of measurements with a transformation to generate output comprising a low-resolution series of measurements, transformation selected from the group consisting of calculating average, counting patterns of motion, switching to frequency domain and alike. In some instances, the apparatus, tangible medium, computer chip, or method further comprises calculating desired activity level values in response to the low-resolution series of measurements. In some instances, the plurality of measurements comprises a plurality of series of measurements and each series of measurements undergoing the step of preprocessing spans a continuous time interval selected from the group consisting of at least 5 seconds long, at least 20 seconds long, at least 1 minute long. In some instances, each series of measurements undergoing the preprocessing operation comprises a high-resolution series of measurements.

In some embodiments, the plurality of measurements comprises a low-resolution series of measurements, and the series of measurements comprising an interval between successive measurements of the series. In some instances, a physical activity level of the subject corresponds to a level of overall physical activity of the subject over a period of time. In some instances, the period of time corresponding to the level of overall physical activity is not less than one tenth of the interval between successive measurements, not less than 5 s, and is not longer than ten times the length of the interval between measurements and is not longer than 1 hour.

In some embodiments, the plurality of measurements comprises a low-resolution series of measurements, and the series of measurements comprising an interval between successive measurements of the series. In some instances, a physical activity level of the subject corresponds to a level of overall physical activity of the subject over a period of time. In some instances, the period of time corresponding to the level of overall physical activity is approximately equal to the interval between successive measurements.

In some embodiments, a physical activity level of the subject during a period of time is selected from the group consisting of an integral characteristic of motion of the subject during the period, a number of specific patterns of motion during the period and a measured physiological quantity of the subject related to an amount of physical activity of the subject during the period. In some instances, the integral characteristic of the subject motion is selected from the group consisting of an average, an area under curve, a total variation, a standard deviation and another similar characteristic of a physical motion signal. In some instances, the integral characteristic is determined in a domain selected from the group consisting of a time domain and a frequency domain. In some instances, the number of specific patterns of motion is selected from the group consisting of a number of steps, a number of steps of a specific type and a set of numbers of steps. In some instances, each number comprises a number of steps of a specific type or alike. In some instances, the steps are classified according to a classification selected from the group consisting of upstairs steps, downstairs steps, walking steps, running steps and alike. In some instances, the measured physiological quantity changes in response to the freely moving physical activity of the subject. In some instances. the physiological quantity is selected from the group consisting of a number of heartbeats, a peripheral oxygen saturation and another physiological parameter measured during an activity level interval, and.

In some embodiments, the wellness parameter is output to a user within about an hour of receiving a last measurement received of the plurality of measurements from the sensor and optionally within about one minute of receiving the last measurement.

In some embodiments, the feature comprises a plurality of features.

In some embodiments, the feature comprises a single feature.

In some embodiments, the feature, or the plurality of features is selected from the group consisting of time domain features, frequency domain features, and transition rates between different activity states.

In some embodiments, the feature or the plurality of features comprises probability distribution properties or occupancy states of the plurality of measurements in a domain selected from the group consisting of a time domain and a frequency domain.

In some embodiments, the feature, or the plurality of features comprise correlation properties of the plurality of measurements in a domain selected from the group consisting of a time domain and a frequency domain.

In some embodiments, the feature or the plurality of features comprise correlation properties of the plurality of measurements. In some instances, the correlation properties comprise autocorrelation in time domain.

In some embodiments, feature or the plurality of features is selected from the group consisting of autocorrelation, power spectral density and transition rates between different activity states and probability distribution properties.

In some embodiments, the transition rates between different activity states comprise a set of transition rates between activity states. In some instances, transition rates between different activity states comprise a matrix of transition rates between the activity states. In some instances, the transition rates between different activity states comprise a full set of transition rates between all activity states. In some instances, the transition rates between different activity states comprise the matrix of transition rates between all activity states.

In some embodiments, the output parameter is evaluated with a combined set of features comprising data from a transition matrix and a power spectral density from the plurality of measurements.

In some embodiments, the tangible medium is configured with instructions selected from the group consisting of preprocessing the plurality of measurements prior to extracting the feature or the plurality of features and post-processing the feature or the plurality of features. In some instances, the preprocessing or post-processing is performed prior to evaluating the wellness parameter.

In some embodiments, preprocessing comprises a procedure to filter the received plurality of measurements according to quality requirements for the wellness parameter being evaluated.

In some embodiments, the apparatus, tangible medium, computer chip or method further comprises preprocessing the plurality of measurements by performing a preprocessing operation.

In some embodiments, preprocessing the plurality of measurements comprises determining whether the received measurements meet a quality requirement; and filtering out the measurements that do not meet the quality requirement.

In some embodiments, the preprocessing operation is selected from the group consisting of down-sampling a series of measurements to lower resolution, calculating a length of vector physical quantity, splitting a series of measurements into slices of fixed or variable duration, filtering out slices of measurements with near-zero activity and logarithm scaling.

In some embodiments, the preprocessing operation comprises a slicing operation that converts the plurality of measurements into a set of slices of predefined length along a time axis.

In some embodiments, the sensor is selected from the group consisting of an accelerometer and a gyroscope and preprocessing the plurality of measurements comprises converting measurements of time evolution of acceleration or rotational quantity along individual axes into measurements of a time evolution selected from the group consisting of an absolute value of acceleration, an angular velocity, an angular acceleration, a rotational speed, and a rotational acceleration.

In some embodiments, extracting a set of features from the plurality of measurements further comprises: quantifying the plurality of measurements into bins of different activity states comprising binned measurements distributed among a plurality of bins; analyzing the binned measurements in response to a statistical distribution among data points of signal levels of the binned measurements in each bin of the plurality of bins; and calculating a transition rate between the signal levels of the measurements in each bin to yield an activity transition matrix. In some instances, the transition rate comprises a feature of the plurality of measurements.

In some embodiments, the postprocessing procedure is selected from the group consisting of imputation of missing or near-zero values, logarithm scaling, and dimensionality reduction.

In some embodiments, dimensionality reduction further comprises linear detrending or principal component analysis decomposition.

In some embodiments, the feature comprises a plurality of features and a single feature is extracted from the plurality of features. In some instances, the wellness parameter is evaluated in response to the single feature.

In some embodiments, the single feature is extracted by dimensionality reduction of the plurality of features.

In some embodiments, the feature or the plurality of features comprises a feature vector and the dimensionality reduction comprises a linear projection of the feature vector onto one or more vectors.

In some embodiments, the wellness parameter comprises a plurality of wellness parameters of the subject, the plurality of wellness parameters selected from the group consisting of the age, the hazard rate, the hazard ratio, the type 2 diabetes status and the body mass index. In some instances, the plurality of wellness parameters comprises a first wellness parameter and a second wellness parameter. In some instances, the second wellness parameter is evaluated in response to a combination of the first evaluated wellness parameter and the feature or the plurality of features

In some embodiments, the plurality of measurements comprises a low-resolution series of measurements comprising a number specific patterns of motion during a period of time. In some instances, an accuracy of the evaluated age corresponds to a Pearson correlation of about 0.55 or higher with actual age for a group of subjects with a uniform distribution of actual age in range from 20 to 70 years old. In some instances, the Pearson correlation is within a range from about 0.55 to about 0.75. In some instances, the number of specific patterns of motion comprises a number of steps of the subject.

In some embodiments, an accuracy of the evaluated age corresponds to a Pearson correlation of about 0.65 or higher with actual age for a group of subjects with a distribution of actual age in range from 20 to 70 years old. In some instances, the distribution comprises a uniform distribution. In some instances, the Pearson correlation is within a range from about 0.65 to about 0.85.

In some embodiments, the accuracy of the evaluated age corresponds to Pearson correlation of about 0.7 or higher with actual age for a group of subjects with a uniform distribution of actual age in range from 40 to 70 years old. In some instances, the Pearson correlation is within a range from about 0.7 to about 0.9. In some instances, the subject is a member of the group of subjects. In some instances, the subject is not a member of the group of subjects.

In some embodiments, the evaluated age of the subject comprises a biological age.

In some embodiments, the evaluated age of the subject is classified among a plurality of classes, the plurality of classes selected from the group consisting of young, adult, old and alike.

In some embodiments, the age is evaluated without inputting an actual age of the subject.

In some embodiments, the wellness parameter comprises the diabetes type 2 status. In some instances, an accuracy of the evaluated diabetes type 2 status corresponds to a sensitivity and a selectivity selected from the group consisting of a sensitivity of at least 0.6 and at a selectivity of least 0.8, a sensitivity within a range from about 0.6 to about 0.9 and a selectivity within a range from about 0.8 to about 0.95, a sensitivity of at least 0.75 and a selectivity of at least 0.75, and a sensitivity within a range from about 0.75 to about 0.95 and a selectivity within a range from about 0.75 to about 0.95. In some instances, the accuracy is determined for a group of subjects. In some instances, the subject is a member of the group of subjects. Optionally, the subject is not a member of the group of subjects.

In some embodiments, the feature is associated with an age of the subject. In some instances, the diabetes type 2 status of the subject is evaluated in response to the evaluated age of the subject combined with a body mass index of the subject. In some instances, the body mass index of the subject comprises the evaluated body mass index wellness parameter or a body mass index input from another source.

In some embodiments, the evaluated diabetes type 2 status of the subject is classified among a plurality of classes, the plurality of classes selected from the group consisting of normal, borderline, diabetic and alike.

In some embodiments, the apparatus, tangible medium, computer chip or method further comprises evaluating hazard rate of a subject with the model in response to the evaluated hazard ratio of the subject combined with a reference hazard rate. In some instances, the reference hazard rate comprises an average hazard rate of a reference population.

In some embodiments, the apparatus, tangible medium, computer chip or method further comprises evaluating hazard ratio of a subject with the model in response to the evaluated hazard rate of the subject combined with a reference hazard rate. In some instances, the reference hazard rate comprises an average hazard rate of a reference population.

In some embodiments, an accuracy of the evaluated hazard rate or hazard ratio is greater than an ROC AUC of about 0.6. Optionally, the ROC AUC is within a range from about 0.6 to about 0.9. In some instances, the accuracy is determined for a group of subjects for which the ROC AUC is determined. In some instances, the subject is a member of the group of subjects. Optionally, the subject is not a member of the group of subjects.

In some embodiments, an accuracy of the evaluated hazard rate or hazard ratio is greater than a concordance index of about 0.6. Optionally, the concordance index is within a range from about 0.6 to about 0.9. In some instances, accuracy is determined for a group of subjects for which the concordance index is determined.

In some embodiments, the hazard ratio comprises a ratio of hazard rates between the subject and a reference hazard rate. Optionally, the reference hazard rate comprises an average hazard rate of a reference population.

In some embodiments, the evaluated hazard rate or hazard ratio comprises a hazard rate or a hazard ratio for 5-year follow up.

In some embodiments, evaluating the hazard ratio comprises evaluating an age-dependent hazard ratio component and an age-independent hazard ratio component of a hazard ratio of the subject. In some instances, evaluating the age-independent hazard ratio component comprises evaluating an age-detrended hazard ratio of the subject.

In some embodiments, evaluating the hazard rate comprises evaluating an age-dependent hazard rate component and an age-independent hazard rate component of a hazard rate of the subject. In some instances, evaluating the age-independent hazard rate component comprises evaluating an age-detrended hazard rate of the subject.

In some embodiments, evaluating the hazard ratio of the subject is performed according to a Cox proportional hazards model.

In some embodiments, evaluating the hazard rate or hazard ratio of the subject is performed according to an accelerated failure time model.

In some embodiments, evaluating the hazard rate or hazard ratio of the subject is performed according to optimization parameters of a Gompertz-Makeham law of mortality.

In some embodiments, the derived parameter is selected from the group comprising signal, information, action or other object evaluated or created or changed or used or transmitted or indexed or delivered in response to evaluated wellness parameter or in response to change in evaluated wellness parameter of the subject.

In some embodiments, the derived parameter is selected from the group consisting of a frailty index, a physiological resilience, a survival function, a force of mortality, a life expectancy, a life expectancy from birth, and a remaining life expectancy, a life span, an average lifespan, a maximum life span, a healthy life span, a health span, a fertile life span, an age when menopause occurs of the subject. In some instances, the derived parameter is evaluated in response to an evaluated hazard rate or hazard ratio of the subject.

In some embodiments, outputting of evaluated wellness parameter is made in the form of adjustment coefficient, or a customized information, content, setting, set of options, service, recommendation, price, term, product or in the form of generation or providing or using or indexation or changing of anything selected from the group: information or object or process, or in the form of triggering or stopping a process.

In some embodiments, the apparatus, tangible medium, computer chip or method, further comprises evaluating a status selected from the group consisting of a type 2 diabetes status and a smoking status of the subject in response to the evaluated hazard rate or hazard ratio of the subject.

In some embodiments, the evaluated life expectancy, or life span, or average life span, or maximum life span, or healthy life span, or health span, or fertile life span, or age when menopause occurs of the subject is classified among a plurality of classes, the plurality of classes selected from the group consisting of short, normal, long and alike.

In some embodiments, the evaluated hazard rate or hazard ratio of the subject is classified among a plurality of classes, the plurality of classes selected from the group consisting of low, neutral, high and alike.

In some embodiments, the evaluated body mass index of the subject is classified among a plurality of classes, the plurality of classes selected from the group consisting of slim, normal, overweight, and alike.

In some embodiments, the apparatus, tangible medium, computer chip or method further comprises evaluating a pregnancy status in response to changes in the body mass index of the subject. In some instances, the change of the body mass index of the subject comprises a change from a first body mass index to a second body mass index greater than the first body mass index.

In some embodiments, the wellness parameter is evaluated exclusively in response to a combination selected from the group consisting of an input gender of the subject, the feature and the plurality of features extracted from the plurality of measurements obtained by the sensor coupled to the subject. In some instances, the wellness parameter is evaluated exclusively in response to a combination selected from the group consisting of feature and the plurality of features extracted from the plurality of measurements obtained by sensor coupled to the subject.

In some embodiments, the sensor comprises a sensor externally coupled to the subject.

In some embodiments, the sensor comprises a non-invasive sensor.

In some embodiments, the sensor is selected from the group consisting of an accelerometer, a MEMS sensor, a MEMS accelerometer, a MEMS gyroscope, a MEMS magnetometer, a pedometer, an optical heart rate monitor and a pulse oximeter sensor.

In some embodiments, sensor does not have electrodes contacting the subject.

In some embodiments, sensor comprises a measuring device. In some instances, the measuring device measures a physical quantity related to physical activity of the subject.

In some embodiments, the measuring device comprises a MEMS sensor. Optionally, the measuring device comprises a plurality of measuring devices.

In some embodiments, sensor comprises a measuring device selected from the group consisting of an accelerometer, a gyroscope, a magnetometer, and a high-precision location sensor or similar sensor. In some instances, the sensor is capable of detecting or measuring an aspect of a physical movements of the subject.

In some embodiments, the measuring device comprises an accelerometer. Optionally, the measuring device comprises of a plurality of measuring devices each of the plurality of measuring devices comprises an accelerometer.

In some embodiments, sensor comprises a measuring device, which measures a physical quantity related to a physical activity of the subject. In some instances, the plurality of measurements comprises readings from the measuring device.

In some embodiments, sensor comprises a set of one or more of measuring devices which measure a physical quantity related to physical activity of the subject. In some instances, the sensor further comprises a processor which transforms the readings from the set of measuring devices. In some instances, the plurality of measurements comprises the transformed readings.

In some embodiments, the transformations, performed by the processor within the sensor, comprise a combination of noise reduction, normalization according to the measuring device calibration, transformation of the raw measurements into physical activity level, detection of the patterns of motion, combining the measurements from several measuring devices into a single quantity and alike.

In some embodiments, sensor comprises a pedometer, a pulsometer, a pulse oximetry sensor or a similar device capable of reporting level of physical activity of the subject or a quantity related to it.

In some embodiments, sensor is embedded into a single physical item designed to be carried by the subject or to be worn by the subject or to be otherwise attached to the subject.

In some embodiments, sensor comprises a sensor of a mobile device.

In some embodiments, the computer chip comprises one or more of measuring devices comprising a sensor.

In some embodiments, the computer chip comprises the sensor.

In some embodiments, the length, the width and the height of the computer chip are within a range from about 1 mm to about 5 mm, about 1 mm to about 5 mm and about 0.5 mm to about 3 mm, respectively. Optionally, the width and the height are within a range from about 0.5 mm to about 2.5 mm, about 0.5 mm to about 0.5 mm and about 0.25 mm to about 1.5 mm, respectively.

In some embodiments, the chip comprises an application specific integrated circuit (ASIC).

In some embodiments, the activity sensor comprises an accelerometer.

In some embodiments, the dimensions of the chip comprise a packaging of the chip.

In some embodiments, the plurality of measurements received from the sensor is transmitted to a remote server. Optionally, the plurality of measurements received from sensor is transmitted to a database of the remote server. Optionally, the plurality of measurements received from sensor is transmitted over the Internet.

In some embodiments, the wellness parameter is evaluated by a processor selected from the group consisting of a remote server and a mobile device configured to be carried by the subject.

In some embodiments, the wellness parameter is provided as an output to a user from an output device in proximity to the user, the user selected from the group consisting of the subject and a user who is not the subject.

In some embodiments, the output is transmitted to the output device from a remote server. Optionally, the output is transmitted to the output device from a database of the remote server. Optionally, the output is transmitted to the output device over an Internet.

In some embodiments, the plurality of measurements from the sensor is transmitted to the remote server over the Internet.

In some embodiments, the output is selected from the group consisting of a visual display, a message, a message in a social network, a message from a chat-bot, a voice, a sound, a haptic device, a brain-computer interface, and a vibration.

In some embodiments, sensor comprises a sensor of a mobile device carried by the subject. In some instances, the user is not the subject.

In some embodiments, the output device is selected from the group consisting of a mobile device carried by the subject, a mobile device worn by the subject, a computer display of a smartphone carried by the subject, a wrist worn device carried by the subject, smart glasses carried by the subject, a smartwatch carried by the subject, and a wristband carried by the subject.

In some embodiments, the mobile device comprises a processor configured with instructions to receive the plurality of measurements from the sensor and to transmit the output to the output device of the mobile device.

In some embodiments, the apparatus, tangible medium, computer chip or method further comprises a second mobile device configured to be carried by the subject, the second mobile device comprising the sensor.

In some embodiments, the sensor comprises a sensor of a smartphone carried by the subject, the smartphone comprising the output device.

In some embodiments, the freely moving physical activity of the subject is a physical activity of a free living subject.

In some embodiments, subject is selected from the group consisting of a human, a pet, a farm animal, and a laboratory animal.

In some embodiments, the plurality of measurements from the sensor comprises measurement from an independent self-supported movement of the subject. In some instances, the independent self-supported movement is selected from the group consisting of crawling, walking, jogging, a movement of the subject over a distance of at least 3 meters, a movement of the subject over a distance of at least 30 meters, and a movement of the subject over a distance of no more than 30 meters.

In some embodiments, the processor comprises a plurality of processors.

In some embodiments, the tangible medium comprises a non-transitory computer readable medium.

In some embodiments, in the apparatus, tangible medium, computer chip or method, the evaluation of wellness parameter of the subject is performed in response to the received plurality of measurements and based on instructions and parameters generated using machine learning techniques for determining the wellness indication for the subject. In some instances, the feature or the plurality of features are extracted according to the instructions and parameters generated using machine learning techniques.

In some embodiments, the method comprises a method for assessing the health and wellness of the subject and the wellness parameter comprise a wellness indication for the subject. The method comprises: providing a plurality of automated evaluation pipelines, each automated evaluation pipeline independently performing the steps of: extracting a set of features associated with the subject from the plurality of measurements; receiving instructions and parameters generated using machine learning techniques for determining the wellness indication for the subject; and processing the extracted set of features and the received instructions and parameters to evaluate the wellness indication for the subject.

In some embodiments, the method further comprises preprocessing the received measurements by performing a preprocessing operation.

In some embodiments, preprocessing the received measurements further comprises: determining whether the received measurements meet a quality requirement; and filtering the measurements that meet the quality requirement by applying transformation to the measurements that meet the quality requirement.

In some embodiments, the preprocessing operation is selected from the group consisting of down-sampling a series of measurements to a lower frequency, calculating a magnitude of acceleration, splitting a series of measurements into slices of fixed or variable duration, filtering out slices of measurements with near-zero activity and a plurality of preprocessing operations.

In some embodiments, the preprocessing operation comprises a slicing operation that converts the received measurements into a set of slices of predefined length along a time axis to reduce computational costs associated with further processing.

In some embodiments, the sensor is selected from the group consisting of an accelerometer and a gyroscope and filtering the measurements comprises converting measurements of time evolution of acceleration along individual axes into measurements of time evolution, absolute value of acceleration, rotational speed, or rotational acceleration.

In some embodiments, filtering the measurements further comprises removing artifacts or outliers from the measurements. In some instances, the transformation is selected from the group consisting of threshold cutoff clipping, frequency band filtering, averaging or smoothing using a moving window, and logarithm scaling.

In some embodiments, each of the extracted features has the same predefined number of feature values.

In some embodiments, extracting a set of features from the measurements further comprises: combining the extracted features into a single set of feature values; and performing an operation to the single set of feature values to yield a final combined feature vector.

In some embodiments, the received measurements comprise high and low-frequency representations. In some instances, combining the extracted features comprises combining features of high and low-frequency representations into a single set of feature values to yield a final combined feature vector.

In some embodiments, extracting a set of features from the measurements further comprises: quantifying the received measurements into bins of different activity levels of activity; analyzing the binned measurements based on determining a statistical distribution among data points of signal levels of the measurements in each bin; and calculating a transition rate between the signal levels of the measurements in each bin to yield an activity transition matrix as a feature of the received measurements.

In some embodiments, the method further comprises post-processing the extracted set of features by performing a post-processing operation.

In some embodiments, the operation is selected from the group consisting of imputation of missing values, logarithm scaling, and dimensionality reduction.

In some embodiments, dimensionality reduction further comprises linear detrending or principal component analysis decomposition.

In some embodiments, the instructions and parameters for determining a wellness indication for the subject are generated by training and validating a neural network using annotated measurements of freely moving physical activity of a plurality of subjects, each of the subjects having a known wellness indication.

In some embodiments, the received measurements comprise high and low-frequency representations. In some instances, combining the extracted features comprises combining features of high- and low-resolution representations into a single set of features.

In some embodiments, the instructions and parameters for extracting evaluating wellness parameter for the subject are generated by machine learning techniques using training and validation set of annotated measurements of freely moving physical activity of a plurality of subjects, each of the subjects having a known wellness indication.

In some embodiments, the method comprises a method of determining a health status of the subject, the wellness parameter corresponding to the health status. In some instances, the health status is selected from the group consisting of the age, the hazard rate, the hazard ratio, the type 2 diabetes status and the body mass index.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of environment in which a system, method, and apparatus can be used according to some embodiments described herein;

FIG. 2 shows examples of different applications for some embodiments of the systems and apparatus described herein;

FIG. 3.1 shows an exemplary categorization of different types of sensors and sensor data;

FIG. 3.2 shows exemplary data obtained according to some embodiments described herein;

FIG. 3.3 shows exemplary embodiments and physical implementation of one or more sensors;

FIG. 3.4 shows an exemplary format for individual series of measurements.

FIG. 4.1 shows exemplified embodiments of the system and apparatus described in the invention;

FIG. 4.2 shows exemplary user interfaces enabled to output one or more wellness parameters and derived wellness parameters;

FIG. 4.3 shows an embodiment of a client-server software-implemented apparatus;

FIG. 4.4 shows an embodiment of a unified software-implemented apparatus;

FIG. 4.5 shows an embodiment of a client-server software-implemented apparatus with integrated sensors;

FIG. 4.6 shows an embodiment of a unified software-implemented apparatus with integrated sensors;

FIG. 4.7 shows an embodiment of a low-level hardware-based apparatus;

FIG. 4.8 shows an embodiment of a hardware-based apparatus;

FIG. 5.1 shows an exemplary overview of the evaluation pipeline.

FIG. 5.2 shows exemplary stages of a feature extraction pipeline.

FIG. 5.3 shows an exemplary procedure that can be used to obtain optimized parameters for the evaluation pipeline disclosed in the invention.

FIG. 5.4 shows exemplary steps for analyzing data collected from a high-resolution sensor.

FIG. 5.5 shows exemplary steps for analyzing data collected from a low-resolution sensor.

FIG. 6.1 illustrates accuracy performance for evaluation of age of a subject in response to high-resolution accelerometer measurements.

FIG. 6.2 illustrates accuracy performance for evaluation of type 2 diabetes status of a subject in response to high-resolution accelerometer measurements.

FIG. 7.1 illustrates accuracy performance for evaluation of age of a subject in response to low-resolution step counter measurements.

FIG. 7.2 illustrates accuracy performance for lifestyle-associated distribution of evaluated hazard ratio of a subject in response to low-resolution step counter measurements.

FIG. 8.1 illustrates accuracy performance for scoring of 5-year followup mortality events by evaluated hazard ratio of a subject in response to low-resolution accelerometer measurements.

FIG. 8.2 illustrates accuracy performance for smoking habit-associated distribution of evaluated hazard ratio of a subject in response to low-resolution accelerometer measurements.

FIG. 8.3 illustrates accuracy performance for diabetes-associated distribution of evaluated hazard ratio of a subject in response to low-resolution accelerometer measurements.

FIG. 9.1 shows a general architecture of an apparatus which comprises a software-implemented system, wherein the front-end module is implemented on a mobile device;

FIG. 9.2 shows a general pipeline of the procedures performed by the module for receiving and accumulating high-resolution plurality of measurements of an apparatus which comprises a software-implemented system;

FIG. 9.3 shows an example screenshot of an output for evaluated age;

FIG. 9.4 shows an example screenshot of an output for evaluated type 2 diabetes status and evaluated body mass index;

FIG. 9.5 shows an example screenshot of an output for evaluated lifestyle-associated hazard ratio (“Health Score”) and its time evolution profile according to embodiments described herein;

FIG. 9.6 shows an example screenshot of an output for an alternative presentation of evaluated hazard ratio (“Lifestyle Hazard Ratio”);

FIG. 10.1 shows a general architecture of an apparatus which comprises a software-implemented system, wherein both the front-end and the back-end modules run on a server;

FIG. 10.2 shows an example screenshot of an output for evaluated age;

FIG. 10.3 shows an example screenshot of a user's percentile rank of physical activity characteristics;

FIG. 11.1 shows an example schematic diagram of an electronic module implementing methods and systems described herein;

FIG. 11.2 shows an example schematic diagram of an electronic module which uses a pedometer, implementing methods and systems described herein; and

FIG. 11.3 shows an example circuit implementation of the apparatus and system described herein.

### DETAILED DESCRIPTION

The method and system disclosed herein comprises receiving data from a non-invasive sensor configured to measure freely moving physical activity of a subject, extracting one or more features from the received data, analyzing and processing the data, and evaluating the data to provide an estimation for the subject's wellness using the extracted features. In some embodiments, sensor data may comprise measurements or a plurality of measurements that can be preprocessed before feature extraction and the extracted features can optionally be postprocessed before a final step wherein the resulting estimation, wellness indicator, set of estimations, or set of wellness indicators, is subjected to an evaluation step, method, protocol, or procedure.

The methods and apparatus disclosed herein are capable of evaluating the wellness parameter of a subject with a model and outputting the wellness parameter in a manner that the subject or another user can monitor or can take action to improve the wellness of the subject. The evaluated wellness parameter of the subject may comprise one or more of an evaluated age of the subject, a hazard rate of the subject, a hazard ratio of the subject, a type 2 diabetes status of the subject or a body mass index (BMI) of the subject.

Some embodiments of the invention may use one or more of the evaluated wellness parameters, also referred to herein as “the primary wellness parameters”, to evaluate one or more derived parameters. The derived parameter may be a wellness parameter, other than the one already evaluated; or a quantity evaluated in response to wellness parameters in a straightforward manner; or a suggested action to improve subject's wellness; or a placement of the subject among groups of subjects; or a rating of the subject relative to a population of subjects or a group of subjects; or a score to be used for personalisation of products and services. The derived parameters can include the wellness parameters such as survival function, life expectancy, life expectancy from birth, remaining life expectancy, life span, average life span, maximum life span, healthy life span, health span, fertile life span, age when menopause occurs, frailty index, physiological resilience, adjustment coefficient for health insurance, tuning parameters for account customization and the like. The derived parameters can include signal, information, action, or other object data evaluated, created, changed, used, transmitted, indexed, or delivered in response to one or more evaluated wellness parameters or a change in one or more evaluated wellness parameters. A range of derived parameters are exemplified herein. Some embodiments of the invention may output one or more of the primary wellness parameters and, optionally, the evaluated derived parameters, while other embodiments may output only the derived parameters. The wellness parameter may be output in the form of an adjustment coefficient, or a customized information, content, setting, set of options, service, recommendation, price, term, product or in the form of generation or providing or using or indexation or changing of anything selected from the group: information or object or process, or in the form of triggering or stopping a process.

Some embodiments of the invention may produce a series of values for a wellness parameter or a derived wellness parameter, separated by at least one day. Each evaluation in this case can be performed in response to a new obtained plurality of measurements and, optionally, the pluralities of measurements of consecutive evaluations can partially overlap. The produced series of evaluated wellness parameter or a derived wellness parameter represent the time evolution profile of the said parameter and can be used to determine trends of subject's wellness. The said identified trends in turn can be associated with changes in personal lifestyle habits or changes in living environment reflecting a continuous gradual change or an event. The trends of time evolution profile of the said parameter can have a time delay in range from one or more days to several months depending on individual traits or age of the subject. Furthermore, inferred associations between trends in time evolution profile of the said parameter and any human interpretable actionable lifestyle metric can be used to provide recommendations on lifestyle managing, lifestyle coaching, lifestyle intervention, changing living environment for the subject. The inferred associations can also be used to monitor wellness status in an individual and personalized manner and to provide recommendations on treatment for the subject with the aim of early detection, prevention and disease interception. The inferred associations can also be used by authorized user for monitoring, managing or customization in corporate wellness, corporate health, and/or insurance.

The methods and apparatus disclosed herein are well suited for combination with many types of externally worn sensors to measure free living activity of a subject. The methods and apparatus can incorporate measurements of freely moving physical activity from one or more of many commercially available devices such as smart phones, smart watches, and externally worn sensors commercially available from many manufacturers such as Apple, Samsung and Fitbit, for example. As such, the methods and apparatus disclosed herein can benefit and improve the wellness of many people.

As used herein, the term “subject” may encompass a moving being such as a person. Although reference is made to measurements on people, the methods and apparatus disclosed herein can be used with many types of subjects.

As used herein, the term “user” may encompass a person who uses a device, apparatus, system, or method as disclosed herein. A user may be a subject, or another person who has a relationship with the subject, such as a friend, a family member, a caregiver, a physician, a person performing a medical, social or other study the subject is participating in, or a person authorized to collect information comprising wellness parameters and/or derived wellness parameters of the subject.

As used herein, the term “processor” may refer to one or more processors used to process data, and can be one or more processors coupled to an activity sensor, or a processor system, and any combination of distributed processors. The processor may also comprise circuitry required for the functioning of a processor or a processor system, such as a memory, an IO controller, wiring and alike. The term “processing circuitry” is used herein interchangeably with the term “processor”.

As used herein, the term “physical activity” may refer to any bodily movement produced by skeletal muscles that require energy expenditure. In some instances, physical activity may refer to the self-sustained motion of the subject's body or of some major part of the subject's body such as, for example, the limb, head or torso.

As used herein, the term “sensor” refers to a device or a set of devices which performs the measurements of the physical activity of the subject and provides data comprising the plurality of measurements received by the invention. In the context of describing the nature of the measured quantity, the term “sensor”, more particular “an accelerometer”, “a gyroscope”, “a pedometer”, “a pulse oximetry sensor”, “a photoplethysmograph” and alike, may refer to a device or a set of devices providing measurements of a particular physical quantity. In the context of describing implementation details of an apparatus, the term “sensor”, more particular “a sensor of a mobile device”, “a sensor of a wristband”, “a sensor embedded into a single physical item” and alike, may refer to a sensor or a set of sensors or a part or parts thereof embedded into a particular object. The entire set of sensors, data from which comprises a plurality of measurements received by an invention, may also be referred to as “a sensor”.

As used herein, the term “measurement” refers to a single value corresponding to a measured quantity at the time of a measurement. The said value can be a single number or, for non-scalar physical quantities, several numbers.

As used herein, the term “hazard” may refer to the risk of an age-related event such as death, or an onset of an age-related disease or condition, including those reported as major causes of death (Global Health Estimates 2016: Life expectancy, 2000-2016. Geneva, World Health Organization; 2018), or menopause.

All these events have a common age-dependent increase of incidence. This is known as the Gompertz law for death (Gompertz, Benjamin. “A Sketch of an Analysis and. Notation Applicable to the Estimation of the Value of Life Contigencies.” Philosophical Transactions of the Royal Society of London 110 (1820): 214-332), was reported for age-related diseases (Shah, Anoop Dinesh, et al. “Type 2 diabetes and incidence of cardiovascular diseases: a cohort study in 1·9 million people.” The lancet Diabetes & endocrinology 3.2 (2015): 105-113; Podolskiy, Dmitriy I., et al. “Analysis of cancer genomes reveals basic features of human aging and its role in cancer development.” Nature communications 7 (2016): 12157) and for menopause (Greer, William. “Preprocessing histograms of age at menopause using the fast Fourier transform.” Maturitas 44.4 (2003): 267-277).

The term “hazard rate” may refer to the probability of hazardous event per unit of time. The term “hazard ratio” may refer to the ratio of estimated hazard rate to the reference hazard rate, wherein the reference hazard rate may be an average populational rate or an average rate in a specific cohort. If hazard rate is related to death it is also referred to as mortality rate and can be used to estimate derived parameters such as survival function, life span, life expectancy, remaining life expectancy, life expectancy from birth, as is known to one skilled in the art of survival analysis and reliability theory. If hazard rate is related to onset of a disease it is also referred to as morbidity rate and can be used to estimate derived parameters such as healthy life expectancy, health span, as is known to one skilled in the art of survival analysis, analysis of disease onset and remission, and reliability theory. If hazard rate is related to menopause, the hazard rate can be used to estimate the expected age when menopause occurs.

The terms system, apparatus and device are used interchangeably in the present disclosure.

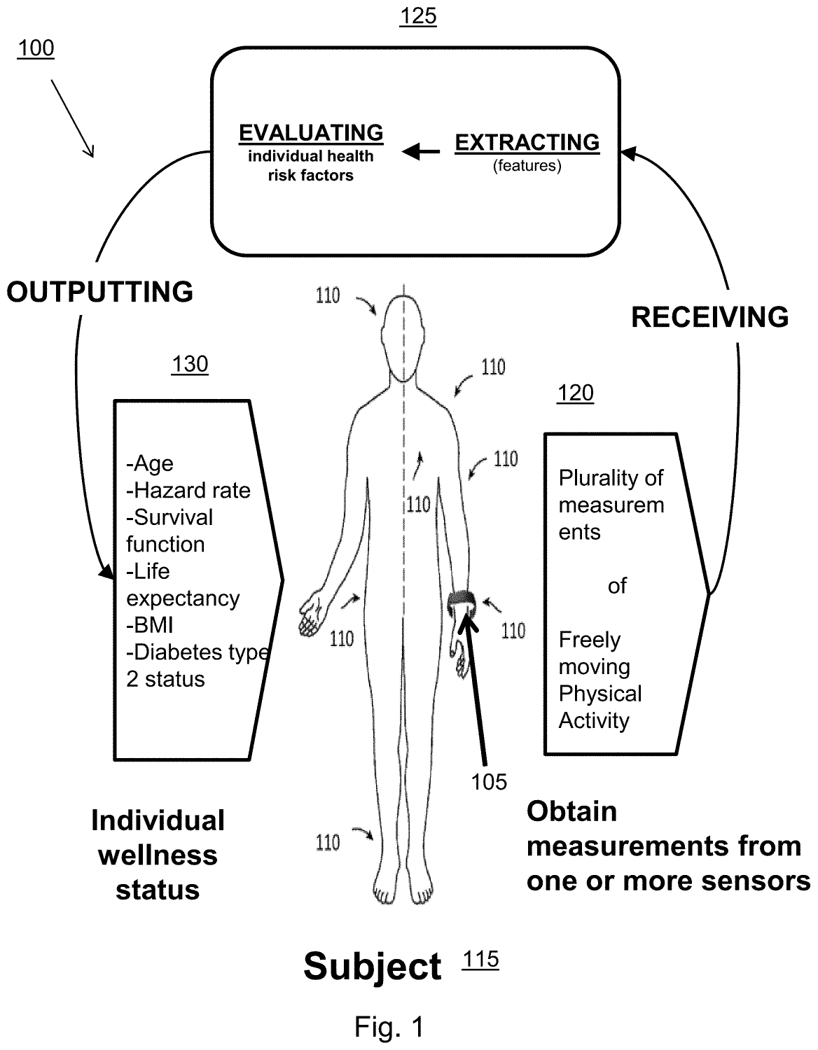

FIG. 1 shows an example system, method, and apparatus according to some embodiments described herein. The system 100 may include an apparatus 105. The apparatus (e.g., wearable device optionally comprising one or more activity sensors 105) described herein may obtain a plurality of measurements 120 from one or more subjects 115. One or more features from the obtained measurements may be extracted and evaluated, according to an evaluation procedure using a model as described herein. The evaluation procedure may yield individual wellness parameters or derived wellness parameters (or estimated wellness parameters or derived wellness parameters) of the subjects or changes in time thereof. Individual wellness parameters or derived wellness parameters may be indicative of the wellness or health status of the subjects, and one or more of the wellness parameters or derived wellness parameters or their changes can be outputted to the subjects, to the users, or to one or more devices. The evaluation procedure may yield a plurality of wellness parameters or derived wellness parameters. The derived wellness parameter(s) may for example be evaluated in response to one or more wellness parameters.

For each subject 115—which may or may not be a human—there may be a characteristic timescale corresponding to the subject's movement. For example, a characteristic timescale may correspond to the typical duration of large-scale movements of major body parts during the subject's self-sustained movement. The characteristic timescale may be used herein to describe data constraints and to categorize sensors. For example, the characteristic timescale for a typical human subject may be taken as the average duration of a step during a fast-paced walk (e.g., 0.5 seconds).

The subject may for example be a human, a pet, a farm animal, a laboratory animal, or the like.

The plurality of measurements 120 from one or more subjects 115 can comprise data originally collected from one or more sensors. In some embodiments, the sensors can convey data to the apparatus in real-time, or near real-time. In other embodiments, data from the sensors can be accumulated and transmitted over to the disclosed system once the amount of obtained data reaches a certain threshold. Alternatively, the plurality of measurements may be obtained from one or more databases.

One or more types of sensors may take measurements from the subject. In some instances, the system can comprise a smart wearable device 105, which can be a smartwatch, smartphone, pocket smartphone, wristband, headband, knee guard, pair of glasses, and the like. The smart wearable device 105 can be externally coupled to an individual subject to measure a freely moving activity of the subject. The smart wearable device 105 can be placed on almost any part of the body 110, and can be coupled to the subject in a variety of ways. For example, the smart wearable device 105 may be placed on the wrist, neck, ankle, chest, waist, head, shoulder, hip, elbow, ear, nose, arm, and legs 110. The wearable device 105 can also be placed inside a pocket of the subject's clothing, or placed inside or integrated into any other object supported by the user.

The smart wearable device 105 may include one or more physical activity sensors that measure a physical aspect of a subject related to the activity of the subject, such as speed, velocity, acceleration, orientation, angular velocity, angular acceleration, longitude, latitude, altitude, heartrate, step rate, peripheral oxygen saturation, galvanic skin response (GSR), etc. and produce activity data regarding the subject. Examples of these sensors include a micro-electromechanical system (MEMS) sensor, an accelerometer, a MEMS accelerometer, a gyroscope, a MEMS gyroscope, a magnetometer, a MEMS magnetometer, a pedometer, an optical heart rate monitor, a heartrate sensor, an electrocardiogram sensor, a photoplethysmograph, a pulsometer, or a pulse oximetry sensor. The sensor may have electrodes contacting the subject. The sensor may not have electrodes contacting the subject.

The sensor may comprise a measuring device. The measuring device may measure or detect a physical quantity related to physical activity or an aspect of physical movement of the subject. The measuring device may comprise a plurality of measuring devices. The measuring device may comprise a micro-electromechanical system (MEMS) sensor, an accelerometer, a MEMS accelerometer, a gyroscope, a MEMS gyroscope, a magnetometer, a MEMS magnetometer, a pedometer, an optical heart rate monitor, a heartrate sensor, an electrocardiogram sensor, a photoplethysmograph, a high-precision location sensor, or a pulse oximetry sensor, or combinations thereof. The plurality of measurements may comprise readings from the measuring device.

The sensor may comprise one or more measuring devices. The sensor may optionally comprise a processor which transformes the readings from the one or more measuring devices. The plurality of measurements may comprise the transformed readings. The transformations may comprise a combination of noise reduction, normalization according to the measuring device calibration, transformation of the raw measurements into physical activity level, detection of the patterns of motion, combining the measurements from several measuring devices into a single quantity and the like.

A computer chip may comprise one or more measuring devices comprising a sensor. A computer chip may comprise a sensor.

One or more features (e.g. a single feature or a plurality of features) from the obtained measurements may be derived from frequencies from activity sensor data. The activity sensor data may comprise frequencies selected from the group consisting of no more than about 0.1 Hz, no more than about 0.01 Hz, no more than about 0.001 Hz, no more than about 0.0001 Hz, no more than about 0.002 Hz, no more than about 0.000011 Hz, and no more than about 0.00001 Hz. The activity sensor data may comprise frequencies of no more than about 100 Hz. The activity sensor data may comprise frequencies of no more than about 1 Hz. The activity sensor data may comprise frequencies within a range consisting of any two of the following values: 0.1 Hz, 0.01 Hz, 0.001 Hz, 0.0001 Hz, 0.002 Hz, 0.000011 Hz, 0.00001 Hz, and 0.000001 Hz.

The activity sensor data may comprise an average power spectral density selected from the group consisting of no more than about 0.1 Hz, no more than about 0.01 Hz, no more than about 0.001 Hz, no more than about 0.0001 Hz, no more than about 0.002 Hz, no more than about 0.000011 Hz, and no more than about 0.00001 Hz.

One or more features may be derived from transitions among activity levels having frequencies selected from the group consisting of no more than about 0.1 Hz, no more than about 0.01 Hz, no more than about 0.001 Hz, no more than about 0.0001 Hz, no more than about 0.002 Hz, no more than about 0.000011 Hz, and no more than about 0.00001 Hz.

The one or more features may comprise probability distribution properties or occupancy states of the plurality of measurements in a time domain or a frequency domain. The one or more features may comprise correlation properties of the plurality of measurements in a time domain or a frequency domain. The one or more features may comprise correlation properties of the plurality of measurements comprising autocorrelation in the time domain. The one or more features may be selected from the group consisting of autocorrelation, power spectral density and transition rates between different activity states and probability distribution properties, or any combination thereof.

In some embodiments, a single feature may be extracted from a plurality of features. The wellness parameter may be evaluated in response to a single feature as described herein. The single feature may be extracted by dimensionality reduction as described herein of the plurality of features. The feature or plurality of features may comprise a feature vector. The dimensionality reduction may comprise a linear projection of the feature vector onto one or more vectors.

The plurality of measurements 120 may comprise data collected from freely moving physical activities of the subjects 115. As used herein, “freely moving subject” or “freely living subject” may generally refer to a subject performing activities, wherein sensors obtain measurements of one or more physical activities of the subjects in a non-invasive fashion. For example, obtaining a plurality of measurements ideally should neither interfere with the subject's activities nor constrain the subject's motion, which means that “freely moving subjects” may not be required to, for example, perform special exercises, undergo examinations or tests, be located in a “special environment”, visit medical or other specialized facilities. The subject may not be in a “special environment” as long as the subject's presence in the building or room is not required, but rather is part of a routine, un-interfered visit. For example, measuring the subject's movements within a building or a room via one or more sensors installed therein may not constitute being in a “special environment”. Similarly, for laboratory animals, equipping an animal cage with a sensor system does not turn the cage into “a special environment” as long as the cage or the animal's movement within the cage is not significantly altered by the sensors. For example, an optical motion detector may be a suitable sensor for the cage if the cage remains structurally unaltered. Splitting the cage in two sections with a sensor-equipped gate significantly alters the cage structure, and may constitute a “special environment”. For a human subject, each sensor may be embedded or integrated into a device, which in turn may be routinely carried by, worn, or otherwise attached to the subject for prolonged periods of time. Examples include, but are not limited to, a watch, smartwatch, fitness wristband, mobile phone, smartphone, or sensor-embedded jewellry or apparel worn by the subject.

Estimated values for one or more wellness parameter can be produced by a sequence of data-transforming operations, which may be collectively referred to as “the evaluation pipeline” 125. Each evaluation pipeline 125 may require a different set of sensors. In some embodiments, data from all required sensors must be present within a received plurality of measurements for a given evaluation pipeline to be applicable. “Evaluation procedure” may generally refer to an implementation of the collection of evaluation pipelines used by an embodiment to produce various estimations for one or more wellness parameter. A set of estimations for various wellness parameters may be produced from a given plurality of measurements by invoking the evaluation procedure.

Individual wellness parameters include, but are not limited to, an estimate of age, BMI, type 2 diabetes status, a hazard rate, and a hazard ratio of a subject based on the plurality of measurements of the physical activity of the freely-moving subject. Estimated age, BMI, type 2 diabetes status, hazard rate, hazard ratio are all essentially based on the same type of data, and thus are hereon collectively referred to as “wellness indications” or “estimations.” The terms “wellness indications” or “estimations” may be used interchangeably.

For each wellness parameter the evaluation procedure may further comprise procedures to determine whenever a valid estimate for the wellness parameter can be produced, and optionally determine the quality of the produced estimate. The determination may be based on a plurality of measurements obtained from one or more sensors. In some embodiments, several types of quality requirements may exist for sensor data as well as several different preferences. These requirements and preferences are disclosed herein, along with other particularities of the plurality of measurements and sensor data.

In some embodiments, the evaluation procedure may not comprise any data quality checks and estimations are always produced. Such estimations are only valid when data quality requirements are actually met. In such embodiments, the user must ensure that the plurality of obtained measurement are of acceptable quality.

In other embodiments, the evaluation procedure comprises several different evaluation pipelines for the same wellness indicator or parameter. The evaluation procedure may further comprise steps to determine which of the evaluation pipelines are applicable. If more than one pipeline is applicable, the procedure may further determine which evaluation pipeline may be the most preferable, and output an estimate of the wellness parameter from the identified pipeline. Alternatively, an embodiment may invoke all applicable pipelines and may output several estimations for the same individual wellness parameter. In some embodiments, the estimations can be accompanied by a preferability and/or quality indication.

In some embodiments, the plurality of wellness parameters may comprise a first wellness parameter and a second wellness parameter. The second wellness parameter may be evaluated in response to a combination of the first evaluated wellness parameter and the feature or plurality of features.

In some embodiments, one or more of the estimations can be outputted via one or more data communication protocols well known in the art, including, but not limited to, Wi-Fi, Bluetooth, I₂C, DART, USB, Ethernet, TCP/IP, Remote Procedure Calls (RPCs), or custom-designed data transmitting protocols over wired or wireless channels. Such embodiments may be part of a larger system. For example, the embodiment may be embedded into a smart apparel or smartphone for enhanced data processing and storage power or may be used as part of a health monitoring system.

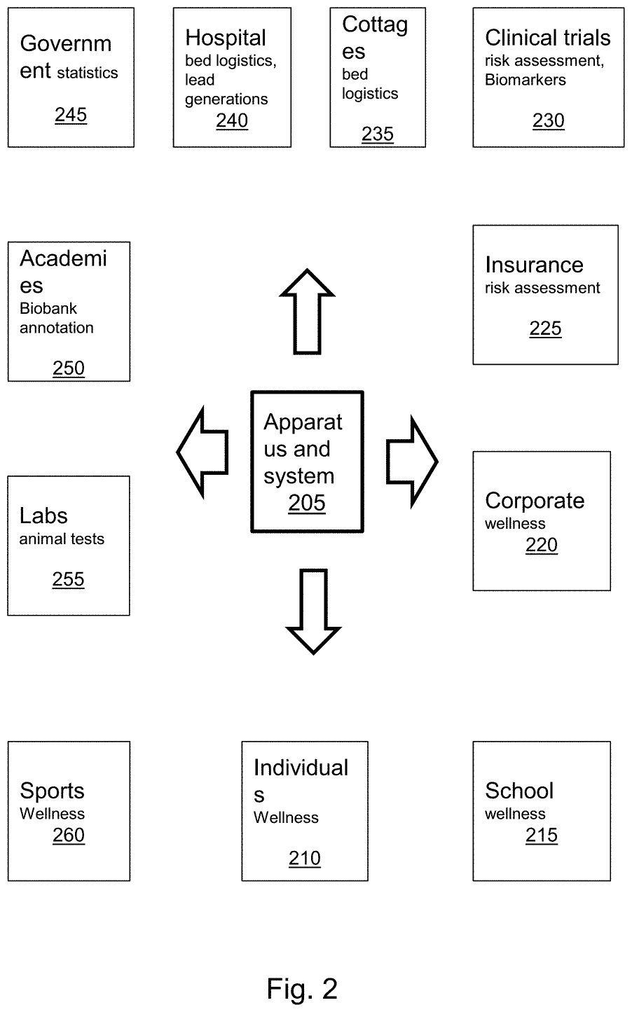

In some embodiments, like the one illustrated in FIG. 1, the apparatus includes a human-oriented interface and conveys one or more of the estimations directly to the person using the invention. The invention may be well-suited for providing estimations to a variety of end users or devices. In some environments, like the one illustrated in FIG. 1, the estimations are conveyed directly to the human subject, wherein the subject is the user of the invention. In other environments, the estimations are used by a third party, for example, by the subject's relative, friend or caretaker, by a representative of insurance company, by a representative of employer or employment agency, by a person or institution authorized to conduct a medical or social study in which the subject is participating, or by a medical service provider or by a government agency. Possible users of the invention are exemplified in FIG. 2.

FIG. 2 shows examples of different applications for some embodiments of the systems and apparatus described herein. The system and apparatus 205 may be applicable in different industries and environments, and for a variety of different purposes. For example, it may benefit individuals 210 by measuring and tracking their wellness indicators. In some instances, students and employees may benefit by tracking their respective wellness indicators at school 215 or at work 220. Other examples may include, but are not limited to, risk assessment in the insurance context 225, risk assessment and biomarkers during clinical trials 230, bed logistics for cottages 235, bed logistics and lead generations for hospitals 240, statistical analysis for the government 245, biobank annotation and statistical analysis for academies 250, animal testing in research labs 255, and wellness and fitness in sports activities 260.

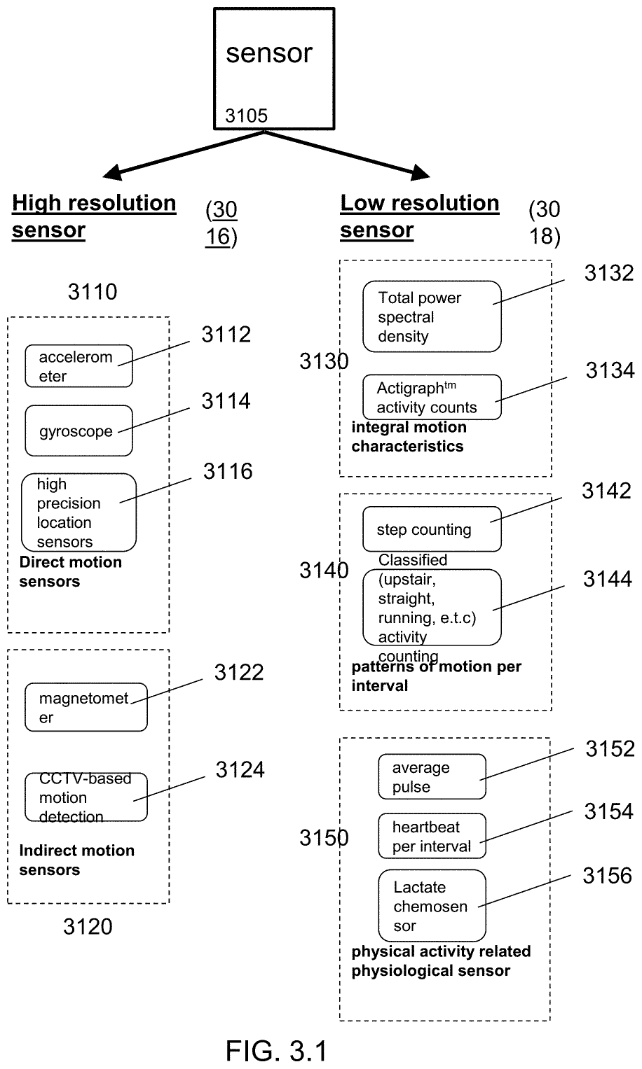

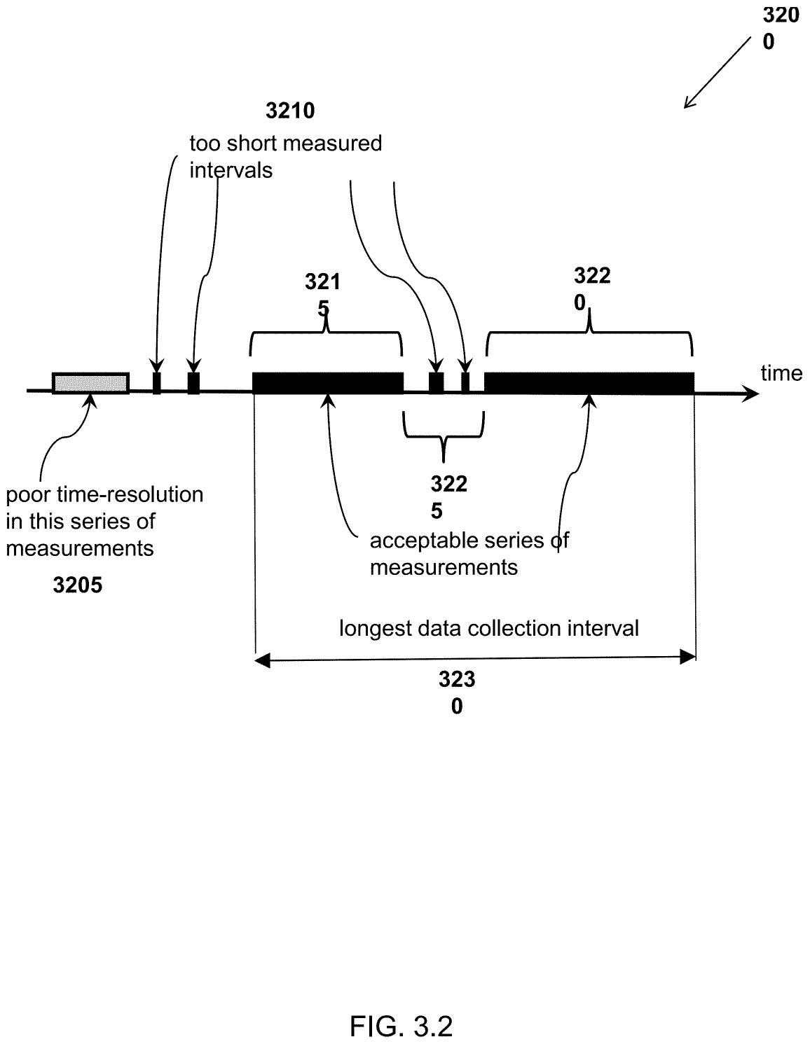

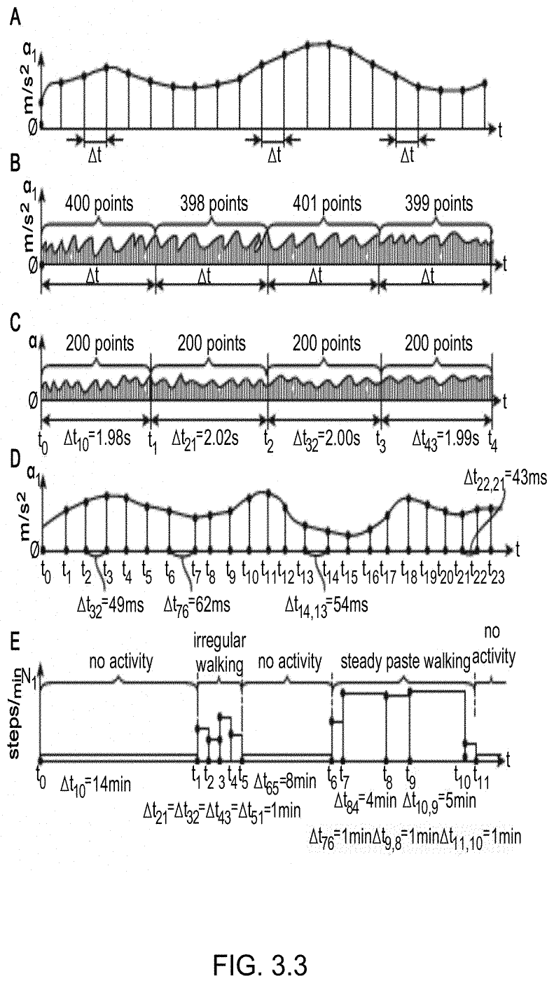

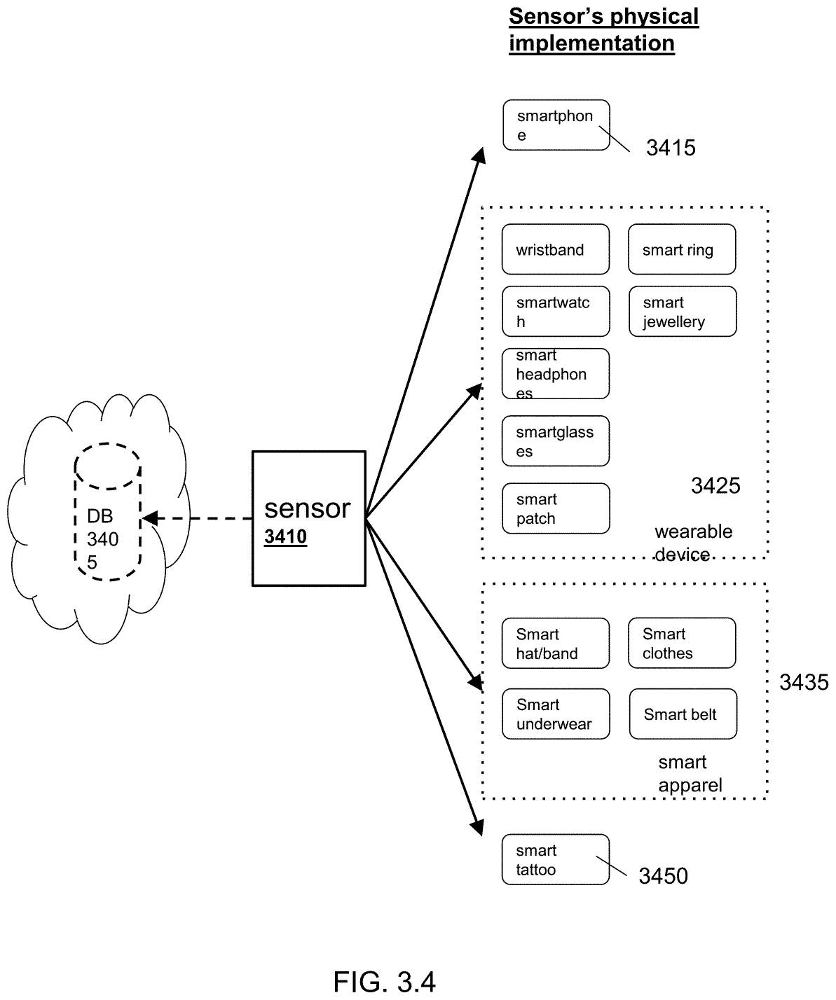

The plurality of measurements comprises one or more series of measurements, each of the said series comprising measurements of some aspect of a physical activity of the subject continuously made by a sensor. Each of the said series, used by the invention, may be categorized into either high-resolution or low-resolution. FIG. 3.1 shows as exemplary categorization of different sensors 3105 according to the type of series of measurements they produce.

High-Resolution Series of Measurements

A high-resolution series of measurements may generally refer to a series of measurements with a time resolution significantly better than the characteristic timescale. A high-resolution series of measurements may be significantly longer than the characteristic timescale. A sensor, producing high-resolution series of measurements, is referred to as a “high-resolution sensor”.

In some embodiments, the system does not analyze the full trajectory, full velocity evolution or similar characteristics of a physical motion, but rather analyzes the patterns of changes within a motion. Since patterns of changes within a motion are relatively similar between different physical quantities, a high-resolution sensor can measure any physical quantity directly related to location, orientation, velocity, angular velocity, acceleration, angular acceleration or to the change thereof. While similarities exist between the patterns of changes obtained from different types of sensors, the system may require an evaluation pipeline specific to, and optimized for, the particular sensor.

Some sensors may fail to produce a series of measurements with resolution and/or duration satisfying the high-resolution sensor criteria. A sensor may be considered “high-resolution” if there are some series of measurements left after dropping the non-satisfactory series of measurements from consideration. For the purposes of applicability and preferability, the non-satisfactory series of measurements can be treated as if they do not exist at all (i.e., null data). Dropping out such non-satisfactory series of measurements is performed at the preprocessing pipeline, which is described later herein.

Exemplary data from a high-resolution sensor are shown in FIG. 3.2. An exemplary time series sensor data 3200 is obtained according to some embodiments described herein. There are four overly short series of measurements 3210 and one series of measurements with resolution too low 3205 for high-resolution sensor in the exemplary sensor data. There are two completely satisfactory series of measurements 3215, 3220 (designated “acceptable series of measurements” in FIG. 3.2). Consequently, a sensor can be used as a high-resolution sensor. In some embodiments, poor series of measurements are completely dropped out from the consideration: the exemplary sensor data will be treated as if there are only two series of measurements (corresponding measured intervals are 3215 and 3220 in FIG. 3.2) separated by a single gap (3225 in FIG. 3.2) and as if there are no data outside the three mentioned intervals. For example, the longest data collection interval 3230 which may be adaptively selected from the exemplary sensor data is depicted in FIG. 3.2.

In some embodiments, the time resolution of high-resolution sensors for a human subject must be better than 250 ms, preferably better than 100 ms, 50 ms, 20 ms. No upper limit exists for the time resolution suitable for the invention. Data with time resolution better than 10 ms are, however, effectively downgraded and processed as if the time resolution does not exceed 10 ms.

For a human subject, a series of measurements from a high-resolution sensor must be longer than 2 s, preferably longer than 5 s, 10 s, 20 s, 60 s, 120 s, 300 s. The time resolution and minimal length of series of measurements for high-resolution sensors for non-human subjects are as described above for the human subject with appropriate scaling using the ratio of non-human to human characteristic timescales.

Examples of high-resolution sensors (e.g. 3016 in FIG. 3.1) attached to the subject and suitable for the invention include 1-axis, 2-axis, and 3-axis accelerometers 3112, gyroscope 3114 and magnetometer 3122. Examples of high-resolution sensors not attached to the subject include motion detectors, such as laser-based, CCTV-based or passive infrared motion detectors 3124. Examples of high-resolution sensor attached to the subject and coupled with an external equipment include a high-precision location sensor 3116, such as a local radio-based location system or a satellite navigation system. Note that the exemplified sensor comes in a variety of designs and only the designs satisfying both the freely-moving requirement and high-resolution time resolution requirements may be suitable as high-resolution sensors.

FIG. 3.3 shows an exemplary format for sensor data comprising one or more series of measurements. In particular, sensor data comprising measurements or a plurality of measurements may further comprise one or more “series of measurements.” Each of the series of measurements can be considered as a continuous period of time for which a sensor actually performs measurements. Such measurements may be reported in a format such that the time evolution of the measured quantity can be restored for the entirety of the said series of measurements. Many such formats are well-known in the art of data science and are all suitable for the use with the method and system described herein.

One example of a suitable format is a time series, i.e a time-ordered sequence of measurements made at some fixed rate, as illustrated in FIG. 3.3(a). In some sensors, the fixed sample rate may in fact slightly vary and an independent high-precision clock is used to determine the actual sampling rate. Such sensors may report the data in the form of modified time series as illustrated in FIG. 3.3(b) and FIG. 3.3(c). Another example of a suitable format is a time-stamped sequence of measurements made at varying rate, as illustrated in FIG. 3.3(d). Yet another example of a suitable format comprise time-stamped reports about a change of the measured quantity, wherein the measured quantity remains constant between the reports, as illustrated in FIG. 3.3(e).

Low-Resolution Series of Measurements

A low-resolution series of measurements may generally refer to a series of measurements of the subject's physical activity level with a time resolution lower than the characteristic timescale but significantly better than 24 hours. A sensor, producing a low-resolution series of measurements, is referred to as a “low-resolution sensor”.

A physical activity level is understood herein as a total amount of physical activity of the subject during some period of time referred herein to as “the activity level period”. An individual measurement within a low-resolution series of measurements can, for example, be an integral characteristic of the subject motion during the activity level period, or a number of specific patterns of motion during the activity level period, or a physiological quantity related to the amount of physical activity of the subject during the activity level period.

An integral characteristic of the subject motion (e.g. 3130 in FIG. 3.1) during the activity level period can be an average, an area under curve, a total variation, a standard deviation or another similar characteristics of the physical motion signal, wherein the signal is considered in the time domain or in the frequency domain. Alternatively or in combination, this integral characteristic of the subject motion during the activity level period can be the amount of time spent in one or more activity states such as sleeping, sitting, heaving a meal, standing, walking periods or the like. Such characteristics may, for example, be generated according to a Human Activity Recognition Dataset as described by [Jorge-Luis Reyes-Ortiz, Luca Oneto, Alessandro Ghio, Albert Sama, Davide Anguita and Xavier Parra. Human Activity Recognition on Smartphones With Awareness of Basic Activities and Postural Transitions. Artificial Neural Networks and Machine Learning, ICANN 2014. Lecture Notes in Computer Science. Springer. 2014]. Herein “signal” is any physical quantity, suitable for the high-resolution sensor as described herein, or a combination thereof, measured during the activity level period.

A number of specific patterns of motion (e.g. 3140 in FIG. 3.1) during the activity level period can be a number of steps, or a number of steps of a specific type, or a set of numbers, each number being a number of steps of a specific type, wherein “a step of a specific type” can refer to one of upstairs steps, downstairs steps, walking steps, running steps and alike.

A physiological quantity (e.g. 3150 in FIG. 3.1) related to the amount of physical activity of the subject during the activity level period can be the number of heartbeats per activity level period, or an average peripheral oxygen saturation level, or another physiological parameter measured during the activity level period that changes in response to the subject physical activity.

The durations of the activity level periods for different measurements need not to be equal. In some embodiments, the activity level periods are of constant duration within individual low-resolution series of measurements, or within all low-resolution series of measurements produced by the same sensor, or with all low-resolution series of measurements within the entire plurality of measurements.

For a human subject, a low resolution series of measurements may have a time resolution lower than 1 s, and optionally between 5 s and 1 hour. The duration of the activity level period can be between 5 s and 1 hour. In some embodiments, the activity level period can be between one tenth and ten times the interval between consecutive measurements within a low-resolution series of measurements. In some embodiments, the activity level period is approximately equal to the interval between consecutive measurements within a low-resolution series of measurements.

The physical activity level of the subject may correspond to a level of overall activity of the subject over a period of time. The physical activity level of the subject during a period of time may be selected from the group consisting of an integral characteristic of motion of the subject during the period, a number of specific patterns of motion during the period, and a measured physiological quantity of the subject related to an amount of physical activity of the subject during the period as described herein. The activity level period may be no less than one tenth of the interval between measurements. The activity level period may be no less than about 5 seconds. The activity level period may be no longer than about ten times the length of the interval between measurements. The activity level period may be no longer tan about 1 hour.

Some embodiments may use the low-resolution sensor with time resolution lower than the duration of activity level period. This may not be a preferred mode of operation but is still acceptable provided that the time resolution and the activity level period duration satisfy the requirements for the low-resolution sensor as described above.

An example of such a non-preferred but acceptable sensor is a sensor that continuously reports the number of a human subject's heartbeats for 30 second intervals every 120 seconds. The 30 second interval is long enough to determine the current the physical activity level and drastic changes of heartbeat rate within a 120 second interval are not typical. Therefore, the exemplified sensor adequately represents the time evolution of the level of subject's physical activity and thus is suitable for the invention.

Any series of measurements not satisfying the requirements for the low-resolution sensor are dropped out from consideration in the same manner as described herein above for the high-resolution sensors.

In some embodiments, the plurality of measurements comprises series of measurements which can be transformed into low-resolution series of measurements. The transformation may comprise calculating average of the signal, counting patterns of motion within the signal, switching to frequency domain, filtering and alike or a combination thereof, wherein the signal is the time evolution of quantity measured by the sensor and represented in the original series of measurements. The said transformation is one of the operations which can be performed within a preprocessing pipeline. In some embodiments, original series of measurements may be high-resolution series of measurements. An embodiment wherein some of the low-resolution series of measurements are obtained via transformation described herein can be treated as if the said low-resolution series of measurements are obtained from some low-resolution sensor directly.

In some embodiments, series of measurements are transformed into low-resolution series of measurements wherein the corresponding physical activity level is an integral characteristic of the subject motion and the original series of measurements represent the suitable signal to be transformed as described herein above. In some embodiments, a desired activity level value may be calculated in response to the low resolution series of measurements.

Some embodiments may comprise a sensor reporting individual steps, or an individual heartbeats. Data from such sensors can be directly transformed into a corresponding physical activity level, specifically, number of steps and, respectively, number of heartbeats per activity level period.

Examples of low-resolution sensors (e.g. 3018 in FIG. 3.1) attached to the subject may include a step counter 3142 a pulse oximetry sensor, a photoplethysmograph, and a heart rate monitor yielding heartbeat counts per minute 3154. Examples of low-resolution sensors not attached to the subject may include passive infrared motion detectors. Other examples of low-resolution sensors may include lactate chemo sensors 3156, average pulse rate detectors 3152, sensors measuring integral motion characteristics 3130 such as total power spectral density 3132, and Actigrapah activity counts sensors 3134.

Many embodiments may comprise one or more low-resolution series of measurements in the received plurality of measurements. In some embodiments, the total combined or accumulated duration of the measured intervals corresponding to the low-resolution series of measurements may be at least about 12 hours, at least about 24 hours, at least about 48 hours, or at least about 78 hours of freely moving physical activity of the subject. The interval between a first measurement and a last measurement of the total accumulated amount of a set of low-resolution series of measurements may be selected from the group consisting of at least about 1 day, at least about 3 days, and at least about 1 week. The total accumulated amount may correspond to freely moving physical activity over a plurality of days. Alternatively or in combination, a portion of the measured intervals between about 8 and about 16 hours after the subjects wake up may be considered. The total accumulated amount of low-resolution series of measurements sampled within the range from about 8 to about 16 hours after the subject wakes up may correspond to at least about 4 hours, at least about 8 hours, or at least about 24 hours of freely moving physical activity. The measured intervals described herein may be obtained simultaneously, sequentially, overlappingly, or with a delay therebetween. The overlapped parts of the measured intervals, if any, may be counted only once or more than once as desired by one of ordinary skill in the art using the teachings described herein.

In some embodiments, the low-resolution series of measurements may be available in the received plurality of measurements. Optionally, the interval between the first and the last measurements may be at least about 1 day, at least about 3 days, or at least about 1 week.

In some embodiments the plurality of measurements may comprise data from more than one low-resolution sensor. In such embodiments the low-resolution data measurement procedures (or requirements) described herein may be applied separately to data from each sensor, to data from a specific sensor, to data from a set of sensors, or any combination thereof.

Some embodiments may comprise one or more of the high-resolution series of measurements in the received plurality of measurements. In some embodiments, the total combined or accumulated duration of the measured intervals corresponding to the high-resolution series of measurements may be at least about 3 hours, at least about 6 hours, at least about 12 hours or at least about 24 hours of freely moving physical activity of the subject. The interval between a first measurement and a last measurement of the total accumulated amount of a set of high resolution series of measurements may be selected from the group consisting of at least about 1 day, at least about 3 days, and at least about 1 week. The total accumulated amount may correspond to freely moving physical activity over a plurality of days. Alternatively or in combination, a portion of the measured intervals between about 8 and 16 hours after the subjects wake up may be considered. The total accumulated amount of high resolution series of measurements sampled within the range from about 8 to about 16 hours after the subject wakes up may correspond to at least about 12 hours, at least about 6 hours, or at least about 3 hours of freely moving physical activity. The measured intervals described herein may be obtained simultaneously, sequentially, overlappingly, or with a delay therebetween. The overlapped parts of the measured intervals, if any, may be counted only once or more than once as desired by one of ordinary skill in the art using the teachings described herein.

In some embodiments, the plurality of measurements may comprise data from more than one high-resolution sensor. In such embodiments the high-resolution data measurement procedures (or requirements) described herein may be applied separately to data from each sensor, to data from a specific sensor, to data from a set of sensors, or any combination thereof.

Some embodiments may comprise a step of preprocessing of the plurality of measurements. Preprocessing may for example comprise filtering out of some series of measurements or parts thereof, or the like. Preprocessing procedures may for example filter out a series or a part of the series of measurements which span a period of time which is too short, or which has an inappropriate time resolution, or which correspond to an activity level period of inappropriate length, or which are otherwise not appropriate for evaluating the wellness parameter as desired by one of ordinary skill in the art. The low-resolution and high-resolution data measurement procedures (or requirements) described herein may be applied to the preprocessed or partially preprocessed plurality of measurements.

Each series of the plurality of measurements undergoing the step of preprocessing may span a continuous time interval. The continuous time interval may be selected from the group consisting of at least 5 seconds long, at least 20 seconds long, and at least 1 minute long.

One or more wellness parameters may be output to a user as described herein. The one or more wellness parameters may be output within about an hour of receiving a last measurement of a plurality of measurements from the sensor. The one or more wellness parameters may be output within about one minute of receiving the last measurement.

FIG. 3.4 shows exemplary embodiments and a physical implementation of one or more sensors. Sensors 3410 may be housed inside smartphones 3415, wearable devices 3425, smart apparel 3435, or smart tattoos 3450. Examples of wearable devices 3425 may include, but are not limited to, wristband, smarting, smartwatch, smart jewelry, smart headphones, smart glasses, smart patch. Examples of smart apparels may include, but are not limited to, smart hat/band, smart clothes, smart underwear, and smart belts. In some embodiments, data from the sensor may be stored in a database 3405. The plurality of measurements may later be obtained from the database, rather than directly from the sensors. Different ways of implementing the systems and apparatus are described herein.

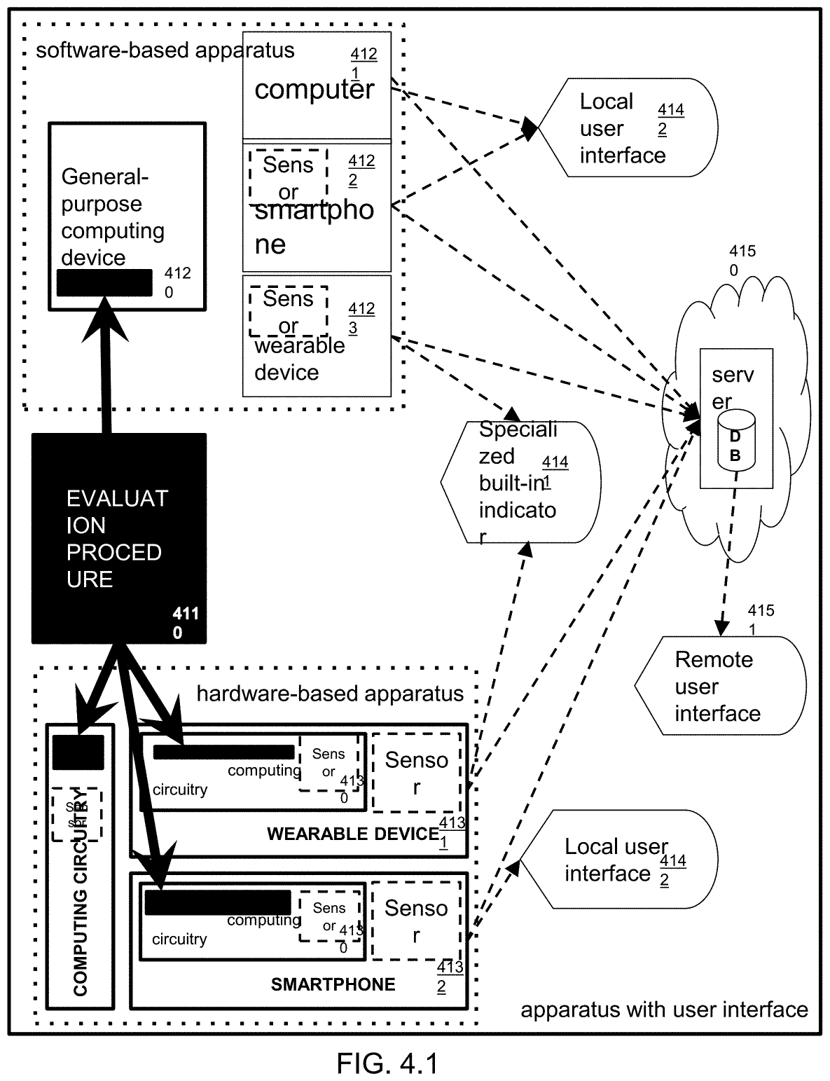

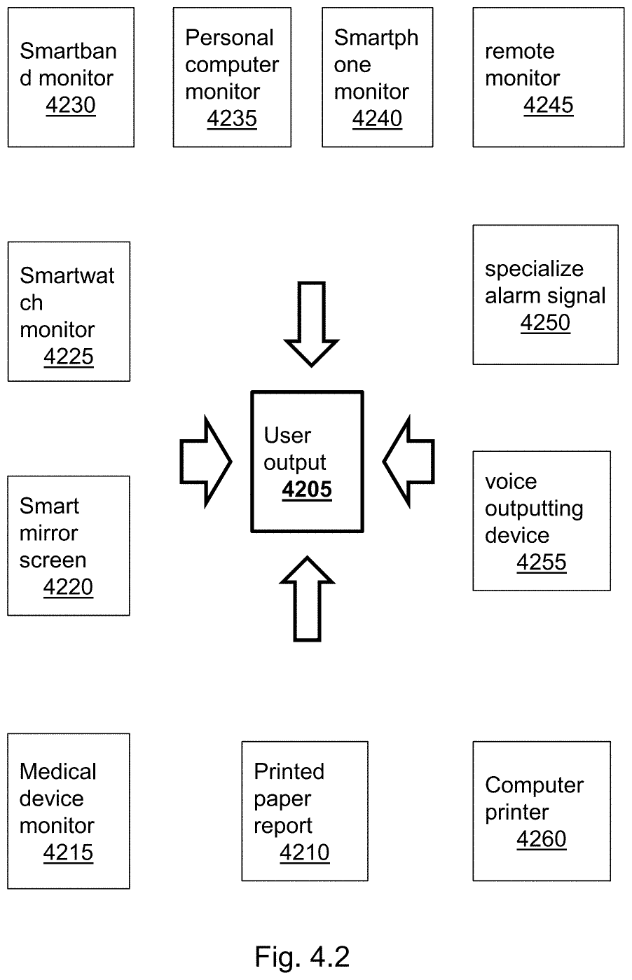

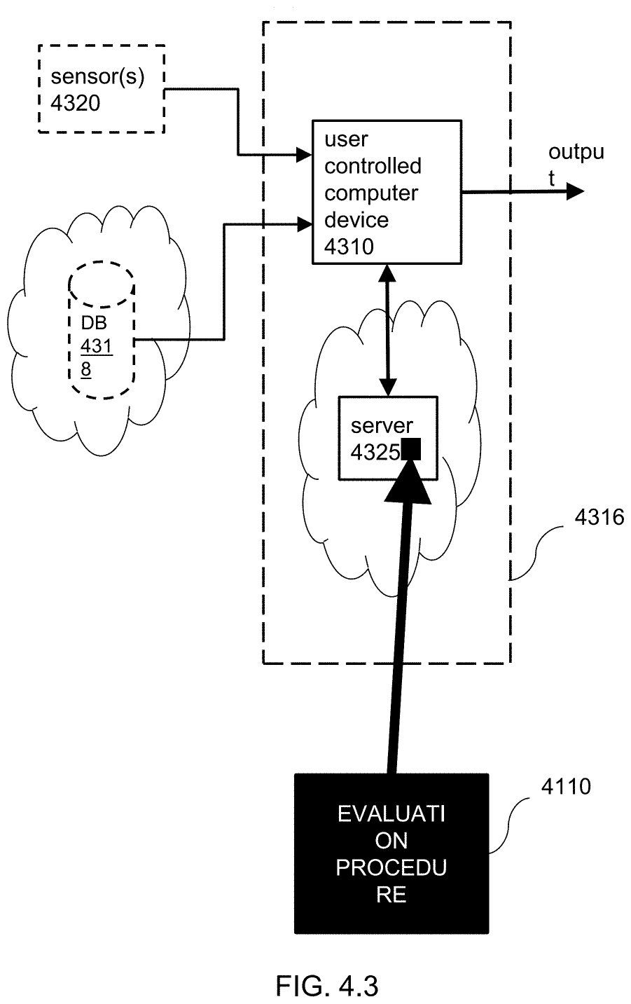

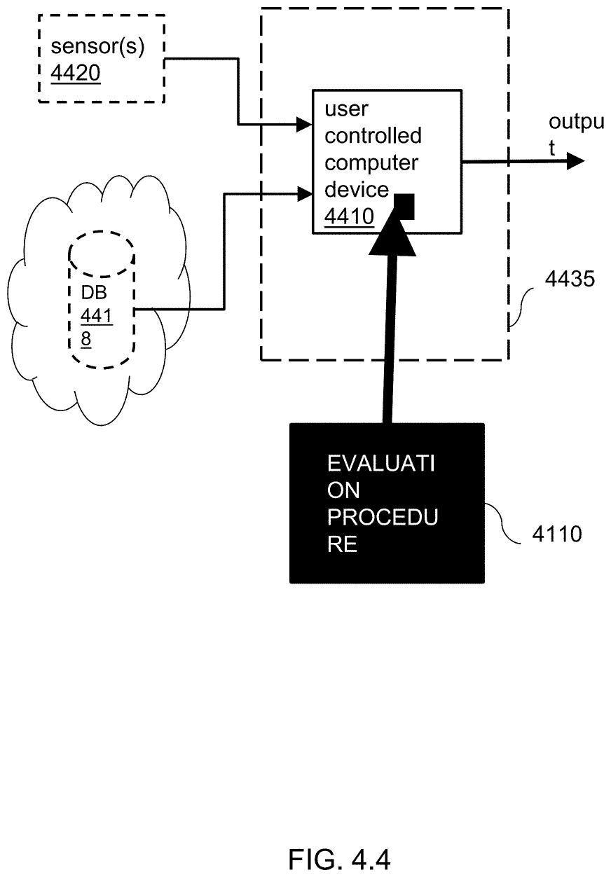

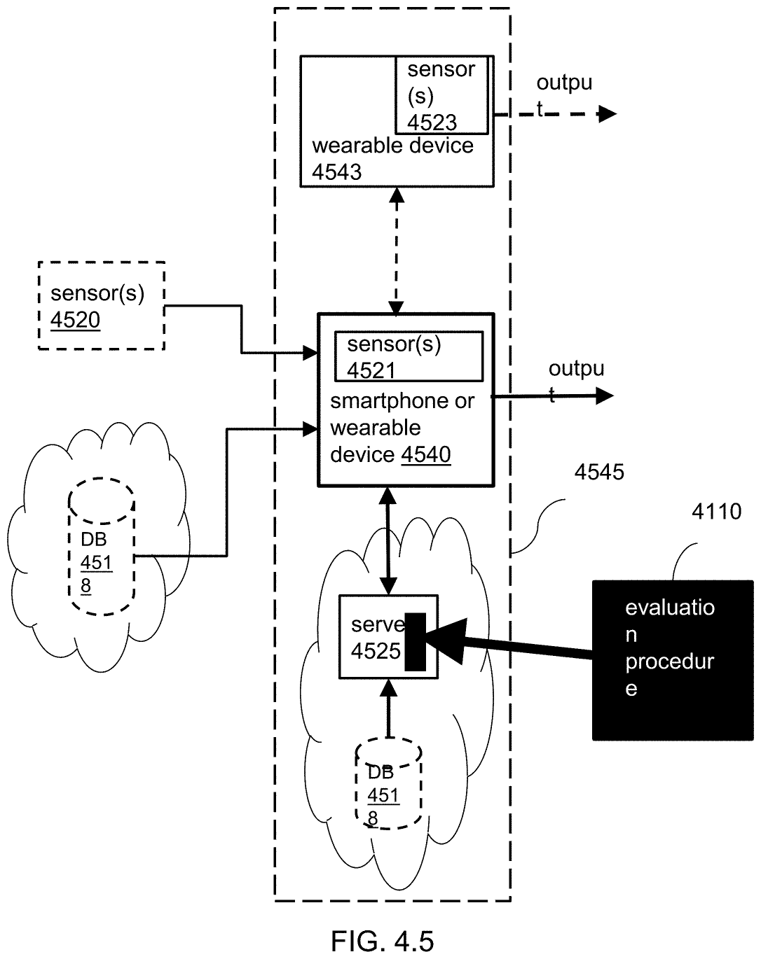

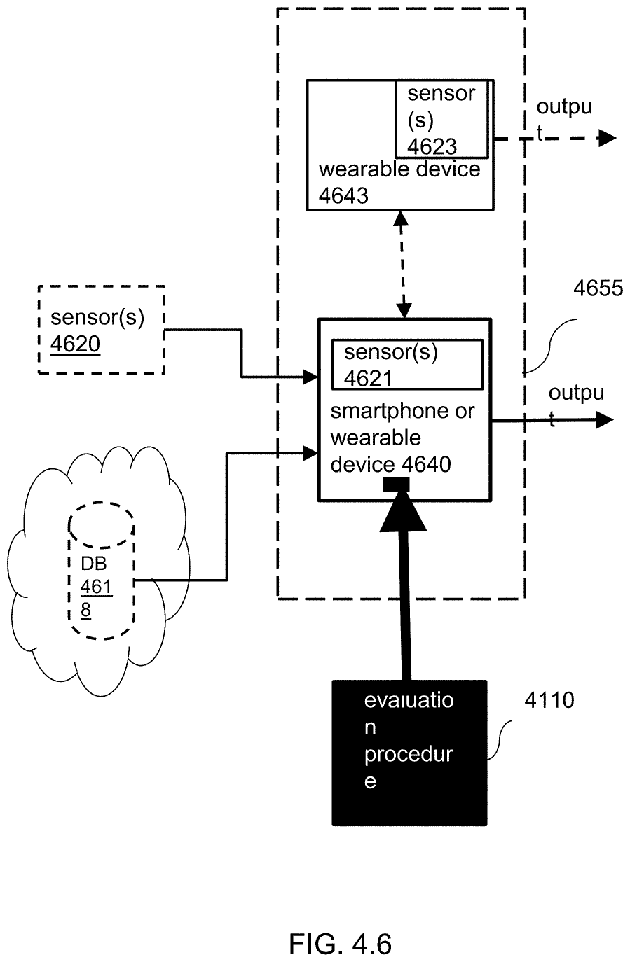

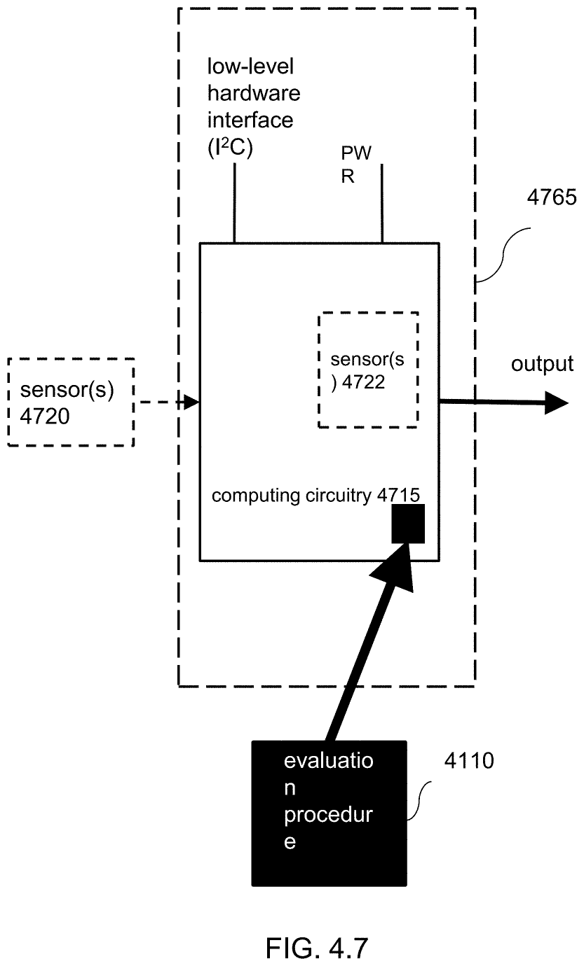

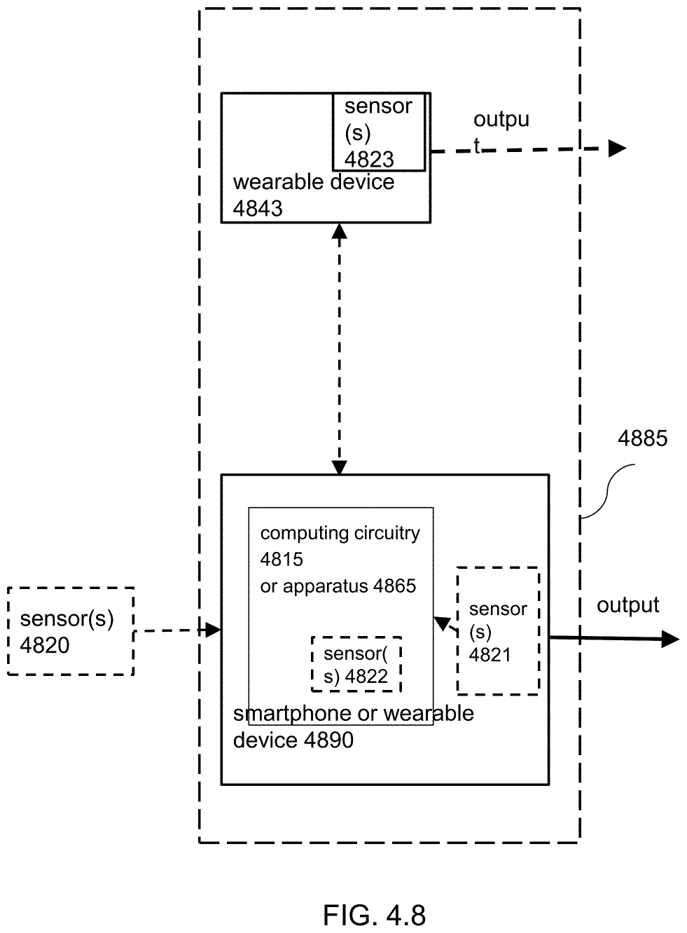

FIG. 4.1 shows exemplified systems in which the apparatus can be implemented according to some embodiments descried herein. The apparatus described herein may be a computing device or a system comprising several connected computing devices. Upon receiving the plurality of measurements, the evaluation procedure implemented within the apparatus may be invoked. The procedure may evaluate one or more estimations (of individual wellness parameters or derived wellness parameters). In some embodiments, the apparatus further comprises a user-interface to convey the estimations to the user. The apparatus may further comprise some or all of the sensors producing the received plurality of measurements.

In some embodiments, the apparatus is implemented as a general-purpose computing device 4120 equipped with a tangible medium upon which the instructions implementing the evaluation procedure 4110 are recorded in the format understandable by the device. The particular examples of such apparatus include an embodiment within a universal computer or a cluster of networked computers, illustrated as 4121, such as a laptop, a nettop, a tablet computer, a workstation, a dedicated server, a virtual computer, a network of computers or a cloud-based computer cluster; an embodiment within smartphone, PDA, smartwatch or other similar carryable or wearable device equipped with general-purpose computing circuitry and not specifically designed for implementing the apparatus, illustrated as 4122; an embodiment within portable or wearable device, illustrated as 4123, wherein the device is specifically designed and equipped with a general-purpose computing circuitry for the purpose of implementing the evaluation procedure 4110 in software form.

The tangible medium may comprise a non-transitory computer readable medium.

In some embodiments, the apparatus is implemented into a computing circuitry 4130, designed specifically for implementing the evaluation procedure 4110. The circuitry 4130 can be implemented in variety of ways, for example, using a digital signal processor, or field-programmable gate array (FPGA) or custom-designed microchip. In some instances the circuitry is implemented on a single microchip, while in other instances several interconnected microchips can be used. The microchip may for example comprise an application specific integrated circuit (ASIC).

As it is well known to the person skilled in the art, there exist many ways to implement the evaluation procedure 4110 in a computing circuitry 4130. In one embodiment, the computing circuitry may comprise the specialized circuitry dedicated for the implementation of the evaluation procedure or some parts thereof. In other embodiments, the computing circuitry may also comprise one or more programmable processors (e.g. a single processor or a plurality of processors) and a non-volatile memory to store the instructions for the said processors implementing the evaluation procedure or some parts thereof. The instructions may be referred to as firmware, which may be essential for the functioning of device, as opposed to a software program or an application, which is a set of instructions enabling optional functionality. Here, firmware may be considered an integral part of the computing circuitry and thus no distinction is made between fully hardware and more flexible firmware-based implementation of the computing circuitry.

While several designs of the computing circuitry 4130 are described with particularities and exemplified herein, the circuitry designs are provided for illustrative purposes only and do not limit the scope of the invention. The person skilled in the art would appreciate the description and examples provided herein and could come up with variety of ways to implement the apparatus in hardware using well-known methods.

Generally, the difference between a computing circuitry implementing the evaluation procedure with a usage of firmware and a general-purpose computing device with a software-implemented evaluation procedure is clear. In some embodiments, however, the system and apparatus described herein may be implemented as a combination of both the software-based and hardware-based implementations. As both software and hardware implemented apparatus are described herein, such borderline implementations are within the scope of the present disclosure.

The computing circuitry 4130 reports one or more of the evaluated wellness indications in a manner suitable for further integration of the circuitry into larger systems. Although such output is not usually suitable for direct observation by the user, it is still a representation of the estimations and therefore the circuitry by itself may constitute an embodiment of the apparatus.

Another hardware-based embodiment of the apparatus, illustrated as 4132, is a smartphone, PDA, smartwatch or other similar carryable or wearable device not specifically designed for the invention, but into which the computing circuitry 4130 is integrated thus providing the said apparatus with one or more estimations. Yet another hardware-based embodiment of the apparatus, illustrated as 4131, is a portable or wearable device specifically designed for the invention, wherein the computing circuitry 4130 is integrated into the device and providing the apparatus with one or more estimations.

In some embodiments, the sensor is not part of the apparatus and the plurality of measurements is transmitted from one or more external sensors to the apparatus via one of the communication methods known in the art, some of which are exemplified herein. One example of such an embodiment is the general-purpose computer 4120.

In other embodiments, one or more of the sensors providing the plurality of measurements are built-in into the apparatus. The embodiment containing the built-in sensor or sensors is not limited to the data from those sensors: the plurality of measurements used by such embodiments may include data obtained from external sensors as well; nor is such an embodiment required to utilize all or even any of the built-in sensors, as long as the suitable plurality of measurements is obtained from external sensors. The optional usage of built-in sensors, such as an accelerometer or a gyroscope built into smartphone, or a pulsometer build into the wristband are illustrated on FIG. 4.1 as dashed rectangles titled “sensor”, as shown inside 4122, 4123, 4131 and 4132.

Some embodiments of the computing circuitry 4130 have a built-in sensor or sensors and may be limited by design to the plurality of measurements provided by said sensors. As a consequence, all hardware-based embodiments of the apparatus implemented using such circuitry are limited to the the built-in sensors.

Other embodiments of the computing circuitry 4130 further comprise an interface for receiving data from external sensor or sensors, and thus the plurality of measurements utilized by such circuitry is not limited to data collected from built-in sensors. Whether external sensors are optional or must be connected to the circuitry may depend on the particularities of the circuitry design and the evaluation procedure implementation. Some embodiments of the computing circuitry 4130 have no built-in sensors or do not use the built-in sensors. Such embodiments receive the plurality of measurements solely from one or more external sensors. The sensor or sensors external to the circuitry may be internal or external, or a combination of both, to the hardware-based apparatus, such as 4131 or 4132, implemented using the above circuitry.

In some embodiments, the external sensors are not connected to the apparatus directly. The plurality of measurements is collected and stored outside of the apparatus (for example on server 4150) and only later is conveyed to the apparatus for evaluating the estimations. Such embodiments are well-suited for processing sensor data collected during large scale medical or social studies. Only an apparatus that allows for the entire plurality of measurements to be received from the external sensors can be used in this manner. Software-based implementations within a universal computer 4121 or computing circuitry 4130 specifically designed for this mode of operation are preferred embodiments for this type of apparatus.

Some embodiments of the apparatus further comprise a user-oriented interface. Examples of such embodiments include a purposely-designed wearable device, such as 4123 or 4131, equipped with a visual or audible or otherwise human-perceivable indication, as illustrated by 4142; a smartphone or a computer, such as 4121, 4122 or 4132, equipped with the software to report the wellness indication via conventional output devices (e.g. a display, a visual display, an audio output, a sound, a haptic output device, a brain computer interface, a vibration, or the like) attached to them; or a system comprising one or more of the apparatus for producing the estimations as described herein together with some remote server 4150, optionally equipped with the database to store the values of the estimations and a device 4141, for displaying the values of the estimations obtained from the said remote server.

The plurality of measurements received from the sensor may be transmitted to the remote server. The plurality of measurements received from the sensor may be transmitted to a database of the remote server. The plurality of measurements received from the sensor may be transmitted over the internet.

The wellness parameter may be provided as an output to a user from an output device in proximity to the user. The user may be the subject or a user who is not the subject. The output may comprise a plurality of measurements. The output may be transmitted to the output device from a remote server. The output may be transmitted to the output device over the internet. The output device may be selected from the group consisting of a mobile device carried by the subject, a mobile device worn by the subject, a computer display of a smartphone carried by the subject, a wrist worn device carried by the subject, smart glasses carried by the subject, a smartwatch carried by the subject, and a wristband carried by the subject.

The sensor may comprise a sensor of a mobile device carried by the subject. The mobile device may comprise a processor configured to receive the plurality of measurements from the sensor. Alternatively or in combination, the processor may be configured to transmit the output to the output device of the mobile device. In some embodiments, a second mobile device may be carried by the subject and optionally comprises a sensor.

The plurality of measurements from the sensor may comprise measurement from an independent self-supported movement of the subject. The independent self-supported movement may comprise crawling, walking, jogging, a movement of the subject over a distance of at least 3 meters, a movement of the subject over a distance of at least 30 meters, and a movement of the subject over a distance of no more than 30 meters.

The apparatus described herein is not limited to the embodiments described above, as where there are other ways to implement the invention. Examples of such ways include implementations where a combination of computing circuitry with a software-based implementation is used or implementations where several networked devices work together as a single apparatus.

FIG. 4.2 shows exemplary user interfaces configured to output one or more wellness parameters and derived wellness parameters. In some embodiments, the apparatus further comprises a means to interact with the user. The main aspect of this interaction is outputting the estimations to the user. A variety of human-oriented interfaces can be used with electronic devices disclosed herein, some of which are illustrated in FIG. 4.2.

The estimations can be conveyed to the subjects visually on a display or a specialised visual indicator, or in audio form using voice in a language of one's choice, or via a combination of any one or more output methods described herein. The voice output can be especially useful for people with impaired vision. Additionally, various signals, such as colored lights, sounds, haptic signals, like vibration, can be used to attract user attention when the new or updated estimations become available and/or when alarming value for the estimation was obtained.

The estimations can be outputted quantitatively, as a numerical value or a range of values; or qualitatively, having a value such as for example, one of “low, neutral, or high” or “good, reasonable, bad, or very bad” or other values provided on a qualitative scale. The estimations can also be outputted as functional or other interpretation in the form of judgments or recommendations.

This in particular provides a way for positioning of the subject against a population based on evaluated age, type 2 diabetes status, BMI, hazard rate, hazard ratio. The estimation may be outputted either on its own or combined with other data on a dashboard, thus providing a better and easy-interpretable presentation of values to the user.

Optionally, the apparatus can receive descriptive metadata of the subject, such as but not limited to geolocation data of the subject. Using such metadata, the estimations can be presented in an augmented environment and in a more comprehensive way and can be used to associate subject's habits and/or lifestyle with wellness indications thus providing actionable recommendations and/or useful scientific information.

Estimations made at different times can be combined to provide the dynamics of the wellness indication. In some embodiments, the estimations can be outputted to the server where estimations made for different subjects are collected. The collected estimations can be used to update positioning of a particular subject within the group of subjects selected according to certain stratification criteria. The stratification criteria can be fixed (e.g., among all subjects, or among subjects with recently obtained estimations, or among the estimations obtained with the same model of the apparatus), or user-specified, or be based on the subject's metadata, or a combination thereof. A score can be outputted directly and/or can be used, optionally with the estimations and optionally with subject's metadata, to obtain an index value like health score, lifestyle score, personal insights or alike. In some embodiments, collected estimations can be annotated with a subject's identity and a comparison of the wellness indications between specific subjects is possible.

To aid user perception and understanding, the outputted values can be displayed in the form of graphs, charts, histograms and similar graphical ways. This form of output can be especially useful to visualize the dynamics of subject's wellness indication; to visualize the subject's wellness indication within some group of subjects or relative to other subjects; to combine different wellness indications together and/or with subject's metadata; or to visualize any combination thereof.

Any form of the outputting described herein can be made using outputting devices integrated within the apparatus used to obtain the estimations, or using one or more external devices, or both. In the latter case, the outputting device may receive data from the apparatus used to obtain the estimations or from the server where the estimation can be collected. Communication methods suitable for connecting the apparatus used to obtain the estimations, the outputting devices, and the server are well known in the art of computer science.

FIGS. 4.3-4.6 show a variety of ways the embodiments described herein can be implemented using the general-purpose computing devices.

FIG. 4.3 shows an embodiment of a client-server software-implemented apparatus.

In some embodiments, as illustrated in FIG. 4.3, the apparatus 4316 comprises a user-controlled computing device 4310 and a server 4325, controlled by a user or by a third party. The user-controlled computing device 4310 is equipped with software that receives measurements or a plurality of measurements, conveys data to the server 4325, receives from the server calculated estimations and outputs the results. The server 4325 may be equipped with software that receives data from the user-controlled computing device 4310, calculates one or more estimations by invoking an evaluation procedure implemented within the software, and conveys the estimations back to the user-controlled computing device 4310. In some embodiments, the apparatus receives the plurality of measurements directly from one or more sensors 4320. In other embodiments, the plurality of measurements is received from the database 4318 where the sensor data are stored.

The connection between sensors 4320, the database 4318, the user-controlled computing device 4310, the server 4325, as well as transmittal of the output, can be accomplished using data transmission methods well known in the art over wired or wireless carrier available on the respective devices.

The choice of the user-controlled computing device 4310 and the server is not described with particularities wherein as the said particularities shall be determined by the person skilled in the art using standard and well-known methods. The user-controlled computing device 4310 is usually a desktop or laptop, but other universal computing devices, for example, tablet computer or a smartphone, are usable a well. The server 4325 can be a dedicated server computer, a cluster of computers, a cloud-based virtual server, a cloud-based cluster and the like.

A person skilled in the art can devise a number of modifications to the apparatus 4316 while still remaining within the scope of the invention. Among such modifications are an embodiment, wherein the evaluation procedure is partially implemented on the user-controlled computing device; an embodiment wherein the user-controlled computing device that conveys the data to the server and the user-controlled computing device that receives the estimation from the server are different devices; an embodiment wherein the output is performed directly by the server; an embodiment wherein some data comprising the received plurality of measurements are received from the database while the rest of the data are received directly from one or more sensors; and various combinations thereof.

FIG. 4.4 shows an embodiment of a unified software-implemented apparatus. In some embodiments, as illustrated in FIG. 4.4, the apparatus 4435 comprises a single user-controlled computing device 4410 equipped with software that receives measurements or a plurality of measurements, calculates one or more estimations by invoking an evaluation procedure implemented within the software, and outputs the calculated estimations. In some embodiments, the apparatus receives the plurality of measurements directly from one or more sensors 4420. In some other embodiments, the plurality of measurements is received from the database 4418 where the sensor data are stored. In some embodiments, some data comprising the received plurality of measurements are received from the database while the rest of the data are received directly from one or more sensors.

The user-controlled computing device 4410 may be implemented in a variety of forms, which can be readily designed by a person skilled in the art given the description provided herein and a particular environment. In particular, while the user-controlled computing device 4410 is logically a single device, physically it may be a single device, a virtual device or a set of interconnected physical and/or virtual devices working together. Among the most common implementations are the implementations in a desktop, a laptop and a server-class computer. Some embodiments of this type are implemented inside smaller and less powerful computing devices, like a tablet computer or a smartphone.

In essence, this type of the apparatus is similar to a client-server software-implemented apparatus 4316 described above and illustrated in FIG. 4.3, with the key difference being the server 4425 controlled by the user and implemented as part of the user-controlled computing device 4410.

FIG. 4.5 shows an embodiment of a client-server software-implemented apparatus with integrated sensors. In some embodiments, the apparatus 4545 as illustrated in FIG. 4.5, comprises a smartphone or wearable device 4540 and a server 4525, wherein the device has universal computing capabilities and further comprises one or more sensors 4521 from which a plurality of measurements is fully or partially obtained. Such an apparatus is a variant of a client-server software-implemented apparatus 4516 described above and illustrated in FIG. 4.3, wherein the smartphone or wearable device 4540 plays the role of the user-controlled computing device 4510. The key aspect to this type of the apparatus are one or more sensors 4521 built-in into the device 4540 and therefore comprise an integral part of the apparatus 4545.

In some embodiments, the plurality of measurements used by the apparatus 4545 further comprises data received from one or more coupled (e.g. externally or otherwise coupled) sensors 4520 and/or the database 4518.

In some embodiments, one or more of the coupled sensors 4520 data from which comprises the plurality of measurements are sensors 4523 built-in into one or more wearable device 4543 purposefully designed for the invention. In such embodiments, the apparatus 4545 further comprises said wearable devices 4543.

As apparatus 4545 is essentially a specialized variant of apparatus 4516, various modifications to the latter, like the one described herein, are also possible to the former. A person skilled in the art shall have no trouble designing some modification specific to apparatus 4545 as well. Among these specific modifications are an embodiment comprising one or more dedicated devices 4543 with sensors 4523, but with no built-in sensors 4521 in the central device 4540; an embodiment wherein the output is additionally or exclusively done from one or more of the wearable devices 4543; an embodiment wherein some parts of the evaluation procedure are implemented within the wearable device(s) 4543; an embodiment wherein there is no distinction between the device 4540 and the device(s) 4543 as all of them are directly connected to the server 4525; and various combinations thereof.

FIG. 4.6 shows an embodiment of a unified software-implemented apparatus with integrated sensors. In some embodiments, the apparatus 4655 as illustrated in FIG. 4.6, comprises a smartphone or wearable device 4640, wherein the device has universal computing capabilities and further comprises one or more sensors 4621 from which a plurality of measurements is fully or partially obtained. Such apparatus is a variant of a unified software-implemented apparatus 4435 described above and illustrated in FIG. 4.4, wherein the smartphone or wearable device 4640 plays the role of the user-controlled computing device 4610. Such apparatus is also a modification of client-server software-implemented apparatus 4545 with integrated sensor illustrated in FIG. 4.5, wherein the evaluation procedure is implemented not within a separate server 4525, but within the smartphone or wearable device 4640.

In some embodiments, the plurality of measurements used by the apparatus 4655 further comprises data received from one or more coupled sensors 4620 and/or the database 4618.

In some embodiments, one or more of the coupled sensors 4620 data from which comprises the plurality of measurements are sensors 4623 built-in into one or more wearable device 4643 purposely designed for the invention. In such embodiments, the apparatus 4655 further comprises said wearable devices 4643.

A person skilled in the art shall have no trouble designing some modification of the apparatus 4645 while remaining within the scope of the invention. Among these modifications are an embodiment comprising one or more dedicated devices 4643 with sensors 4623, but with no built-in sensors 4621 in the central device 4640; an embodiment wherein the output is additionally or exclusively done from one or more of the wearable devices 4643; an embodiment wherein some parts of the evaluation procedure are implemented within the wearable device(s) 4643; and various combinations thereof.

The computing circuitry 4715 implementing the evaluation procedure is schematically illustrated in FIG. 4.7. FIG. 4.7 shows an embodiment of a low-level hardware-based apparatus. The circuitry may either have one or more built-in sensors 4722, or be able to receive data from one or more external sensors 4720, or both. As the said circuitry produce the estimations it constitutes an embodiment which is herein referred to the low-level hardware-based apparatus 4765.

The computing circuitry 4715 has some form of a power connection and one or more low-level hardware interfaces to output the produce estimations and (when needed) to receive data from external sensors 4720. The interface(s) may also have additional functions, such as providing control over the computing circuitry and/or sensor(s), allowing direct access to the sensor(s) data, updating firmware, monitoring the performance and/or health of the circuitry, and the like. These additional functions are typical and well-known in the art of hardware engineering and are not described further herein. I²C is a widely used interface and may be used for the invention. Other hardware interface(s) applicable for the particular circuitry design may also be used.

The preferred embodiment of the low-level hardware-based apparatus 4765 is in the form of a single microchip, even more preferably in the form of a single microchip wherein the plurality of measurements is fully obtained from sensor(s) 4722 built into said microchip.

The microchip may be configured to be carried by the subject. The microchip may comprise dimensions including a length, a width, and a height. The dimension of the chip may comprise a packaging of the chip. The length may be no more than about 5 mm. The length may be within a range from about 1 mm to about 5 mm. The length may be within a range from about 0.5 mm to about 2.5 mm. The width may be no more than about 5 mm. The width may be within a range from about 1 mm to about 5 mm. The width may be within a range from about 0.5 mm to about 2.5 mm. The height may be no more than about 3 mm. The height may be within a range from about 0.5 mm to about 3 mm. The height may be within a range from about 0.25 mm to about 1.5 mm.

In some embodiments, the low-level hardware-based apparatus is a multipurpose apparatus designed for providing the estimations and some additional function. For example, the low-level hardware-based apparatus can be designed as a replacement for a smartphone sensor microchip and therefore it provides raw sensor data typically expected from such microchip in addition to the estimations, with the used plurality of measurements not necessarily comprising data from all sensors.

FIG. 4.8 shows an embodiment of a hardware-based apparatus. A key aspect of the hardware-based apparatus is that the evaluation procedure is implemented within the purposely designed computing circuitry.

One of the possible embodiments of the hardware-based apparatus 4885 is a smartphone or wearable device 4890 comprising the low-level hardware-based apparatus 4865 is illustrated in FIG. 4.8. In some embodiments, the invention the low-level hardware-based apparatus 4865 receives data from sensors other than the built-in sensor 4822. In such embodiments, the additional sensor(s) are either the sensor(s) 4821 built-in into the smartphone or wearable device 4890, or an external sensor(s) 4820.

Another possible embodiment of the hardware-based apparatus, also illustrated in FIG. 4.8, is a smartphone or wearable device with integrated computing circuitry 4815, wherein the computing circuitry 4815 is not an independently usable apparatus 4865. One example of such an embodiment is a smartphone therein the computing circuitry 4815 is integrated with some other smartphone circuitry in one microchip. Another example of such an embodiment is a smartwatch wherein all functionality including the computing circuitry 4815 is implemented on a single chip. Yet another example of such an embodiment is a smartphone wherein the computing circuitry 4815 is mostly implemented on a separate microchip, but also requires some other smartphone circuitry, usually memory, for operation.

Similar to the software based apparatus with built-in sensors described above, in some embodiments of the hardware-based apparatus 4885, the external sensor(s) are sensors 4823 built into one or more wearable device(s) 4843 purposely designed for the invention; in such embodiments the apparatus 4885 further comprises said wearable devices 4843.

Method

The method disclosed in the present invention can perform evaluation of wellness parameters of the subject, and optionally can perform evaluation of derived wellness parameters. A device, apparatus, or system comprising one or more sensors, can be used to take one or more measurements or a plurality of measurements. The measurements or plurality of measurements can undergo a series of processing or transforming operations to produce an output. Outputs include, for example, any single or combination of related or unrelated predictions, estimations, one or more wellness parameter, or evaluations of wellness parameter of the subject. Examples of evaluated wellness parameters include the subject age, hazard rate, hazard ratio, BMI, and type 2 diabetes status. Optionally, derived parameters can be evaluated including the said wellness parameters and Survival Function, Life Expectancy, Remaining Life Expectancy, Life Expectancy From Birth.

The method disclosed herein comprises one or more methods for analyzing and processing signal data from sensors on a device, system, or apparatus. The methods comprise receiving the measurements or plurality of measurements followed by extracting one or more features from the received measurements or plurality of measurements. Further steps can comprise evaluating the estimation for a subject's wellness parameter using the extracted features. In some embodiments, the data comprising the measurements or plurality of measurements can be preprocessed before feature extraction and the extracted features can optionally be postprocessed before a final step wherein the resulting estimation, wellness indicator, set of estimations, or set of wellness indicators, is subjected to an evaluation step, method, protocol, or procedure.

Preprocessing, feature extraction, postprocessing, and evaluation of the estimations or values are collectively called herein “the evaluation pipeline.” In many embodiments, feature extraction is a mandatory part of method. Preprocessing and/or postprocessing may be optional steps of the evaluation pipeline. The evaluation pipeline may be automated.

The Evaluation Pipeline

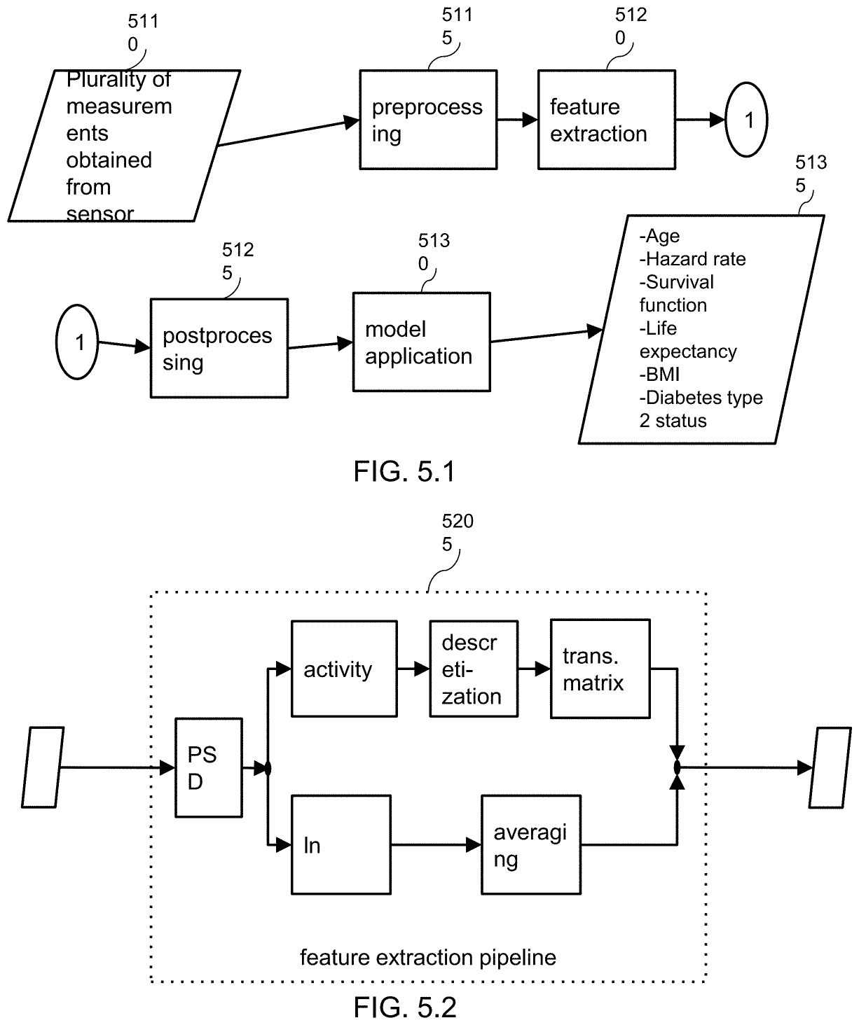

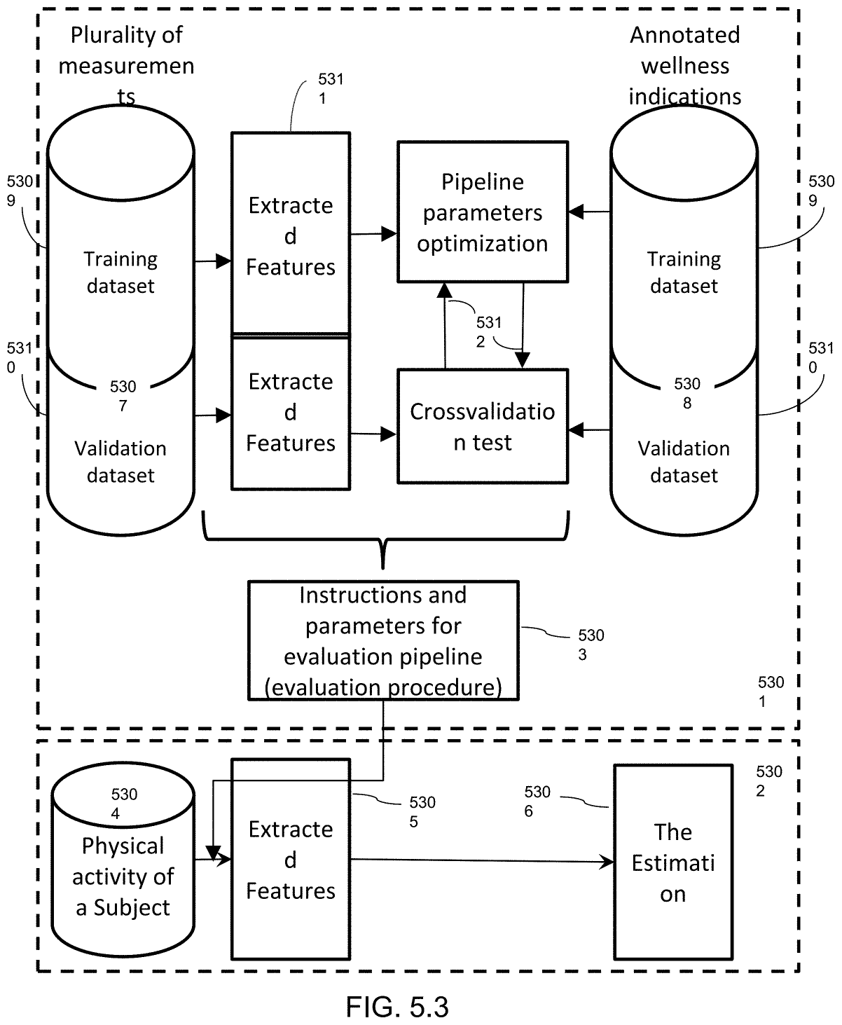

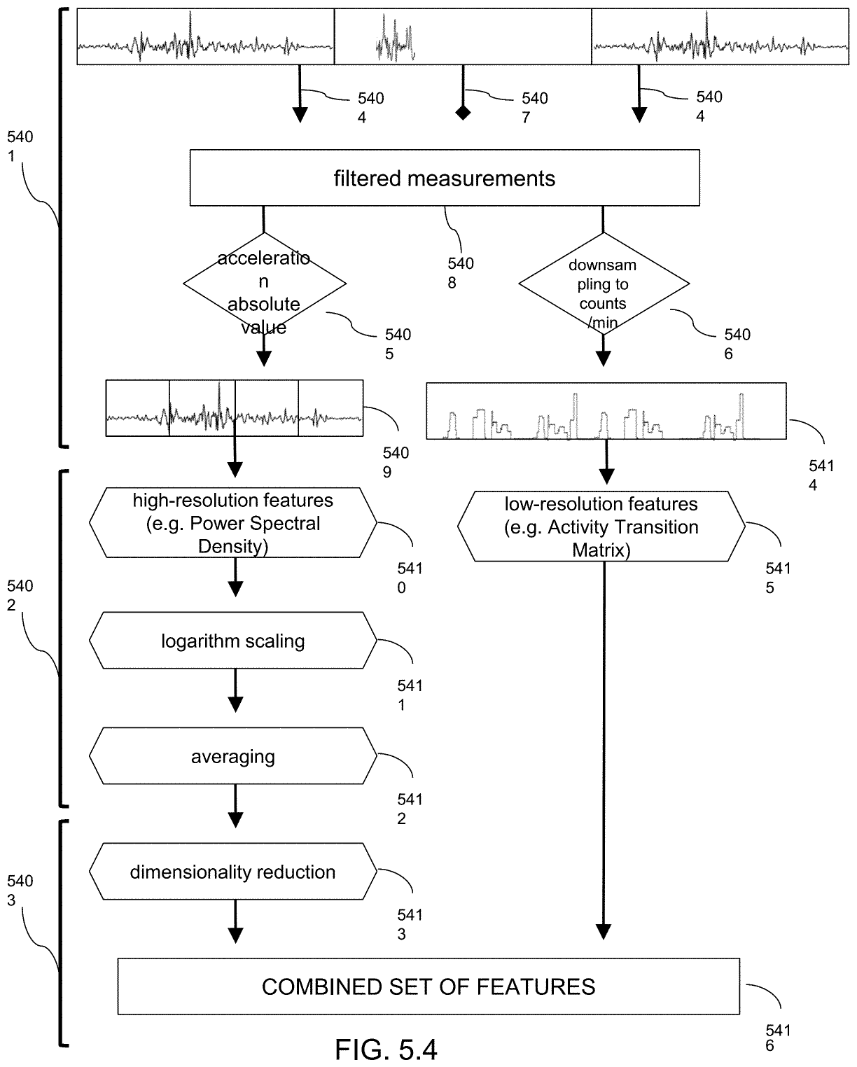

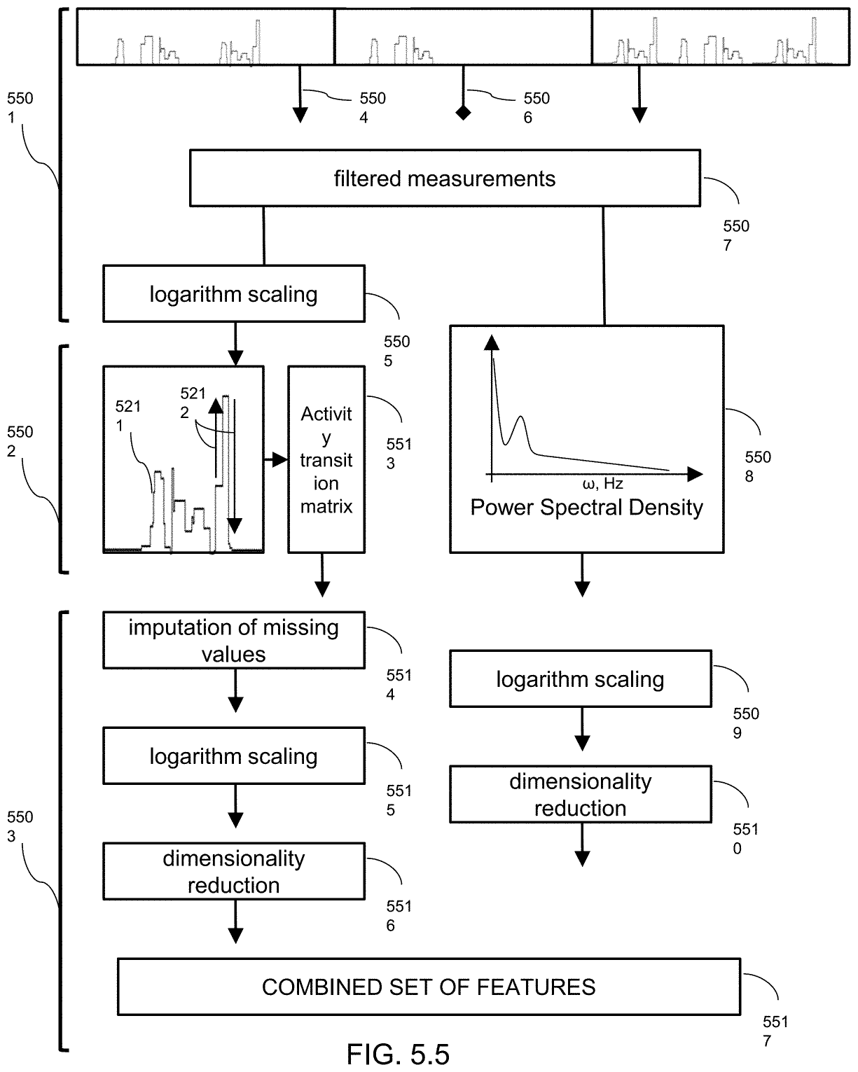

The evaluation pipeline as shown in FIG. 5.1 can comprise a number of operations. The operations can be grouped into a set of stages and when executed can produce an evaluation of a wellness parameter in response to the measurements of plurality of measurements or a feature extracted from the plurality of measurements as described herein. In some embodiments sensor data, measurements, or plurality of measurements 5110 can first be subjected to an optional preprocessing stage 5115. Following the preprocessing stage 5115, the data can undergo feature extraction stage 5120. After the feature extraction stage 5120, the data can further undergo optional post-processing operations 5125. The evaluation of a wellness parameter or a derived wellness parameter can then be performed by the model application stage 5130 using features resulting from the postprocessing pipeline. The model is a set of instructions to perform computational operations to produce a value of evaluated wellness parameter in response to features and as generally understood by one skilled in the art of data sciences the model can comprise a set of parameters that need to be optimized to achieve a reasonable evaluation accuracy level prior to the practical application of the said model. Each of the stages in the method can be a directional data flow graph with each node performing a computational operation (e.g. a mathematical transformation).

The model application stage can perform an evaluation of a wellness parameter in response to a set of features resulting from the feature extraction stage, which in turn can comprise an optional preprocessing stage, the feature extraction stage itself, and an optional post-processing stage. This separation of the feature extraction stage into three stages is conventional. This is to reflect the fact that the evaluation pipeline can comprise a set of optional operations prior to and after the feature extraction which does not change the physical meaning of input data. In some embodiments of the invention the evaluation pipeline can comprise preprocessing operations of filtering out invalid pluralities of measurements, conversion of values of acceleration along individual spatial axes into value of acceleration vector and alike, all the said operations resulting in output data of the same physical meaning as the input. In contrast, the operations of the feature extraction stage itself can change output data of physical meaning different from the input, for example outputting values of power spectral density as the features of an input time series of acceleration values, thereby converting the input time domain data into frequency domain output. In some embodiments of the invention the evaluation pipeline can comprise postprocessing operations of converting the features to logarithmically or exponentially scaled or averaged features, reducing dimensionality of the features and alike, all the said operations resulting in output data of the same physical meaning as the input.

The evaluation pipeline comprises a set of instructions specific for the wellness parameter to be evaluated and specific to the type of the received plurality of measurements. The exact content of the measurements or plurality of measurements 5110 submitted to the evaluation pipeline can vary and the evaluation pipeline can comprise instructions to perform preprocessing tests and filtering operations to ensure whether the received plurality of measurements are valid for performing the evaluation of the wellness parameter. Variations in the type and quality of data can be present within the measurement, within or across measurements, or within or across a plurality of measurements 5110. The received plurality of measurements can be different in: the amount of data collected, the time-duration of the series of measurements, the data formats due to the sensor model, and the settings and/or individual characteristics. In many embodiments of the invention, the evaluation pipeline can comprise feature extraction or model application operations that require certain sampling resolution of the input plurality of measurements. In this case the preprocessing stage can comprise operations for resampling of the received plurality of measurements and operations to test whether such resampling can be made. For example, if the received plurality of measurements consists of high resolution series of data sampled at 50 Hz and the evaluation pipeline requires low resolution plurality of measurements with sampling rate 1/min, the downsampling can be performed. In contrast, if the received plurality of measurements consist of measurements sampled once a day, the preprocessing stage can reject the data because it is unlikely that it can be resampled to 1/min rate.

Preprocessing can include other data transformations: splitting the series of measurements into series of measurements without gaps, splitting a series of measurements into slices of predefined (e.g. fixed or variable) duration, filtering out slices of measurements with near-zero activity, logarithm scaling, calculating a length of vector physical quantity, calculating a magnitude of acceleration, evaluating the total amount of data available within the received measurements or plurality of measurements, and dropping out, removing, or disregarding data fragments of unsatisfactory quality, or a plurality of preprocessing operations. In many embodiments of the invention, the evaluation accuracy depends on the total accumulated amount of data, because an averaging of extracted features is required to filter out noise as can be appreciated by one skilled in the art of data sciences. In that case those pluralities of measurements which do not meet the requirement on minimal amount of accumulated data can be rejected because the evaluation pipeline cannot guarantee the evaluation accuracy. The total amount of data is understood as the total amount of time of a series of measurements excluding the gaps between the series of measurements. In many embodiments of the present invention, the requirement on minimal amount of total accumulated data refers to measurements performed during 8 to 16 hours after the subject woke up which can be determined by time stamps or by the time distance from first activity level above a predefined threshold during a day.

The feature extraction stage produces a set of numerical characteristics of the received and optionally preprocessed plurality of measurements. The said characteristics, commonly referred to as features or descriptors in the art of data sciences numerically describe certain properties of the data samples and in a preferred mode of the present invention can describe correlation properties and statistical distribution properties of the measurements of the received pluralities of measurements. The correlations properties for either low-resolution or high-resolution series of data can be the values of autocorrelation function at one or more time scales, or a power spectral density of the plurality of measurements at one or more frequencies. The autocorrelation function and power spectral density represent the autocorrelation properties of the plurality of measurements in time domain and in frequency domain, respectively, and are essentially the same set of features.

The statistical distribution properties can describe the relative density of different activity level states or relative density of transition rates between the said activity level states. For a low-resolution series of data the activity level states can be time periods with a different number of steps or number of other patterns or motives of motion extracted from the data or a level of overall activity categorized or binarized into bins with predefined activity level thresholds. The statistical distribution properties of transition rates between the said different activity level states can comprise the full set of transition rates between all activity states and in this case can be represented in the form of transition matrix, wherein each off diagonal bin in column j and row k corresponds to a transition rate from activity state j to activity state k. For example, if the plurality of measurements can be represented in the form of step counts per minute, activity state j can be all minutes with less than 50 steps and activity state k can be all minutes with 50 or more step counts. If the transition matrix is represented in the form wherein the diagonal elements are the negative sum of transition rates in each row, the transition matrix comprises the essential characteristics of both the statistical distribution of activity states and the correlation properties of the plurality of measurements.

The optional post-processing stage can comprise operations of averaging, scaling, dimensionality reduction, input of missing or near-zero values of the extracted features. The said transformations can be required to convert the features to the form of post-processed features suitable for the model application stage. Averaging can be required to transform a set of features of the same time and size extracted for separate slices of the series of data into an average feature representation for the fully received plurality of measurements. Dimensionality reduction can be performed using principal component analysis or other methods of data science and can be required for filtering out noise components in the extracted features. Scaling may be required to bring the features of plurality of measurements of similar physical quantity to the same scaling.

The plurality of measurements may comprise a series of data. The series of data may comprise a time series, with data separated by a time within a range selected from the group consisting of one millisecond to one second, one millisecond to one minute, one millisecond to one hour, one millisecond to one day, five seconds to one minute, five seconds to one hour five seconds to one day, one minute to one hour, and one minute to one day.

The plurality of measurements may comprise a series of measurements. The series of measurements may comprise a low-resolution series of measurements, a high-resolution series of measurements, or any combination thereof. A low-resolution series of measurements may represent a low resolution time evolution profile of physical activity levels of the subject with a time resolution longer than 1 s and optionally with the time resolution within a range from 5 seconds to 1 hour. A high-resolution series of measurements may represent a fast time evolution profile of a physical quantity related to motion of the subject with the time resolution shorter than 1 s, for example shorter than 250 ms. The shortest time resolution of the plurality of measurements may be about 0.01 s. The shortest time resolution of the plurality of measurements may be about 0.05 s. Each high-resolution series of measurements may have a time resolution selected from the group consisting of short than 100 ms, shorter than 50 ms, and shorter than 20 ms.

The feature extraction pipeline shown in FIG. 5.2, 5205, can transform the optionally preprocessed data 5115 from the measurements or plurality of measurements 5110 into a set of features. The set of features can comprise an ordered set of one or more numbers containing all information from received measurements or plurality of measurements used by the particular evaluation pipeline to produce the values or estimation. The format of the set of features can be fixed for a particular embodiment of the evaluation pipeline. The set of features can be a single mathematical vector and can comprise a plurality of integers, real or complex scalars, vectors, matrices, tensors and/or other numerically-represented mathematical objects. The terms “plurality of features”, “set of features” and “the features” are used interchangeably herein.

The model application pipeline can transform the set of features of fixed format into a numerical value for the estimation. In some embodiments, the quality of the produced estimation can be determined based on the received measurements or plurality of measurements. In such embodiments, the model application pipeline can further comprise transformations of the set of features into an assessed quality value.

In some embodiments of the invention, the format of the set of features produced by the feature extraction pipeline and the format of the set of features used by the model application pipeline are different. The postprocessing pipeline can transform the former into the latter. The term “feature vector” describes a mathematical representation of the set of features and can apply to any fixed-format representation of the received measurements or a measurements or plurality of measurements within the evaluation pipeline, e.g. any postprocessed “feature vector” can also be a “feature vector.”

It should be noted that although feature extraction and the model application are described as distinct stages, the distinction between them can be somewhat arbitrary. The convention used herein for delineating separate stage in the evaluation pipeline is that the model application pipeline comprises nodes with parameters optimized using data science and/or machine learning methods, the preprocessing and postprocessing pipeline comprise nodes which do not change the physical meaning of the transformed values, and the rest of the nodes belong to the feature extraction pipeline.

Some embodiments may utilize a convolutional neural network architecture of the model—these provide examples of evaluation pipelines wherein the boundary between feature extraction and model application pipelines is fuzzy. That is, both pipelines contain parameters obtained or estimated by data science and/or machine learning methods and there can be no parameter-free transformations in between them. A convolutional neural network is a type of feed-forward artificial neural network that would be understood by one of skill in the art. A convolutional neural network can comprise nodes. Nodes can comprise parameters that govern their computation.

Obtaining Parameters for the Evaluation Pipeline

The model application stage comprises an evaluation model which can produce an evaluation of wellness parameter in response to numerical features of the plurality of measurements using a modeling method of data sciences. The present invention discloses that different models can yield evaluation of comparable accuracy, starting from the most simple linear regression, to more complex models relying on logistic regression, support vector machine, random forest, neural network architecture, provided that the extracted features capture the essential information on statistical distribution and correlation properties of the plurality of measurements, as exemplified in the experimental section below. The model is a parametric set of instructions wherein the optimized values of the parameters need to be provided so that the evaluation accuracy of the model is reasonable. One skilled in the art of data sciences will appreciate that the model parameters are optimized through a machine learning approach using a dataset comprising both the pluralities of measurements of physical activity of a set of subjects and the known values of the wellness parameter to be evaluated. An annotated set of measurements or pluralities of measurements can be used to obtain a set of optimized model parameters that may be generalized to a large set of subjects and provide evaluated or predicted wellness parameters for a subject for which the value of wellness parameters are not known.

The disclosure provided herein describes a variety of exemplary evaluation pipelines well-suited for the invention. Methods of machine learning used to obtain the parameters for the said pipelines are also described. The applications of the described methods for evaluating particular wellness indications are further exemplified with details necessary for practical implementation within the devices, systems, and apparatuses described herein.

It is to be understood that the embodiments presented herein are examples, and that other embodiments of the method can be used with or in place of the described evaluation pipelines and measurements or plurality of measurements. Other methods may comprise any of the disclosed steps or processes as well as any other well-known in the art of data science. One of skill in the art would understand that a variety of modifications to an evaluation procedure can be made without significantly affecting the results, therefore such modifications remain within the scope of the disclosed inventions.

Results obtained using a particular evaluation pipeline can be reproduced with similar precision using a variety of different evaluation pipelines. For example, a dimensionality reduction followed by a linear model can be replaced with a two layer artificial neural network, and the Fourier transform followed by dropping high-resolution coefficients can be replaced with a downsampling followed by the Fourier transform. The replacements in this examples are not mathematically identical, but are acceptable in many practical applications. A lot more of such practically acceptable or even mathematically identical replacements are well-known and are described in the data science literature, and any of these can be used in combination with or in place of the steps disclosed herein. Such replacements can be used to derive a different embodiment of the method from the evaluation pipelines described herein and still remain within the scope of the invention.

FIG. 5.3 illustrates a general scheme for obtaining an evaluation model with optimized set of model parameters based on a dataset of plurality of measurements annotated with known values of the wellness parameter to be evaluated 5301. Pipeline for deriving the trained and validated model are depicted in the dashed box labeled 5301. Applications of the instructions and optimized parameters of the evaluation pipeline derived based on the training and optionally on the validation dataset for making evaluations for a new subject, based on newly received measurements or a plurality of measurements of the physical activity of the said subject, are presented in the dashed box labeled 5302. The illustrated process is based on a machine learning approach that can utilize a reference training dataset of measurements of physical activity of a group of subjects (e.g. an annotated or labeled training set) wherein each subject is annotated or labeled with the actual value of parameter to be evaluated. In many embodiments, the process illustrated in 5301 is performed first to obtain an implementation of the evaluation pipeline and a set of instructions and corresponding parameters 5303 are optimized during the machine learning process; with the operations defined in 5302 performed independently after the optimized parameters are obtained. In some embodiments, the process illustrated in 5301 is repeated or iterated to create an updated implementation of the invention based on a substantial amount of additional reference data, instructions, and parameters are accumulated. In some embodiments, the instructions and parameters 5303 optimized in 5301 may be applied to measurements or a plurality of measurements of physical activity for each new subject 5304, and with the full pipeline of feature extraction 5305 and evaluation stages 5306 illustrated in 5302. In further embodiments, model performance may be measured by the process in 5302.

The implementation or model building steps as shown in 5301, illustrate machine learning approaches to building a model that can produce instructions and parameters for the evaluation pipeline. A training dataset comprising measurements or a plurality of measurements 5307 of physical activity of subjects can be annotated by one or more of wellness indications 5308 that are to be estimated by the method. In many instances, the training dataset can be further split into training 5309 and validation or testing dataset 5310 to control for parameters including for example overfitting. Model overfitting is an undesirable characteristic of a model that is well known by those in the field of data science and/or machine learning. Features can be extracted according to a feature extraction pipeline 5311 and a machine learning approach can be used to fit its parameters to optimize the estimation in terms of an appropriate accuracy metric. Parameters of the feature extraction pipeline 5311 can be optimized during the machine learning process. For example, the exact number of bins can be optimized if the feature extraction pipeline comprises calculation of an activity level transition matrix. In many instances, the machine learning optimization process can be carried out through iteration or repeatedly 5312 until convergence (e.g. when the model attains an optimal value of accuracy metric for both the training and the validation or testing datasets). In many instances, where the wellness indication to be estimated is a continuous value, such as Age or BMI, a preferred metric is a correlation or a root mean squared error. These metrics or others may be used. In instances where the wellness indication to be estimated is a labelled, sorted, or ranked value, such as type 2 diabetes status or hazard event (e.g. mortality event) status, a preferred metric can be enrichment, evaluated as the area under a receiver operating curve (ROC AUC), or concordance, a selectivity-sensitivity metric (e.g. ROC AUC, precision recall, or the like).

The machine learning approach can be either linear or non-linear, and one skilled in the art of data science would appreciate that the choice of approach depends on the balance between the number of parameters to be fit and the number of individual subject samples in the training dataset as is exemplified in particular implementations described herein.

Feature Extraction Pipeline

FIG. 5.4 illustrates general aspects of a feature extraction pipeline that can be applied to measurements or a plurality of measurements obtained from a high-resolution sensor, which provides both high-resolution and low-resolution series of data. FIG. 5.4 provides an illustrative description of a feature extraction pipeline that can comprise a set of operations that can be organized in series or in parallel. Operations can be grouped in a sequential pipeline of pre-processing 5401, feature extraction 5402, and post-processing 5403. Operations can be run in parallel for both the high-resolution measurements, downsampled low-resolution, and acquired low-resolution measurements. In some embodiments the exact order and number of operations can differ depending on the estimated value and on the type of sensor from which the measurements are obtained.

First, in the preprocessing step 5401 measurements or a plurality of measurements are fed into the system. The system can then perform a set of preprocessing operations (5404 and 5407) to determine whether the measurements meet the quality requirements. A signal or measurement may pass the initial quality check or quality filter 5404 and move on, as indicated by the arrow, or the signal or measurement may not pass the initial quality check and not move on 5407 as indicated by the diamond. The quality check operations or quality filter 5404 are performed to ensure that a valid estimation can be performed. The signals or measurements that successfully move through the quality filter 5404, proceed to a filtering step where the signals or measurements are filtered 5408. The signal or measurements may in parallel be transformed into acceleration absolute values 5405 in one process flow, and downsampling to counts (e.g. per minute counts) 5406 in another process flow. The resulting signal or measurement may then be further processed (see 5409 and 5414) before entering the feature extraction pipeline 5402.

In some embodiments the quality checks 5404 can be performed to determine the interval length. Intervals can comprise various lengths, including full days, and there can be requirements, including threshold cutoffs for a minimal duration of an activity period during the day and/or for a minimal amount of total activity or a maximum activity level. In some embodiments, the minimal requirement can be that physical activity is recorded such that the measurements cover an interval longer than ten hours either continuously or intermittently. For intermittent intervals, a nearly uniform distribution can be required such that a set of measurements can be obtained in each consecutive hour, or two hours, or three hours, or three or more hours.

In some instances, a signal may not meet the criterion of a particular quality check and thus, does not make it past a quality check or filtering step. For example, the measurement processed by quality control 5407 does not meet the minimal requirements and does not pass into, or pass the quality filter 5408, while other measurements e.g. the measurement processed by 5404, does meet the minimal requirements and does make it through the quality filter 5408 and into further estimation procedures.

In some instances, at least several days of measurements are required to achieve statistically confident averaging for estimation of age, BMI, type 2 diabetes status, hazard rate or hazard ratio. In some instances, the number of required days is greater if measurements have significant gaps. In some instances, the minimum number of required days is 7 days or 28 days.

One or more transformation operations are applied after the quality filter 5404. In some embodiments, a high-resolution sensor is a tri-axial accelerometer or gyroscope and a transform operation converts measurements of time evolution of acceleration along individual axes into measurements of time evolution, absolute value of acceleration, rotational speed, or rotational acceleration 5405. This facilitates implementation of a method that is independent of orientation of the sensor with respect to either the subject or the direction of Earth's gravity. In some embodiments, additional operations are applied including but not limited to: threshold cutoff clipping, frequency band filtering, averaging or smoothing using a moving window, and logarithm scaling. These operations are useful to treat certain measurements, artifacts, or outliers that can appear or result in measurement errors or defects. For example, if a sensor is known to have a growing or decaying trend and in other aspects provides valid measurements, a frequency band filter can be applied to filter out any frequencies below the daily circadian rhythm.

Preprocessing operations in some embodiments can include a slicing operation 5409 to convert the measurements or plurality of measurements into a set of measurements or slices of predefined length along a time axis. This preprocessing step can reduce computational costs of following operations and permit different processing methods on the slices which can exhibit different activity levels.

The feature extraction operation 5410 can perform extraction of numerical or ordinal characteristics of the preprocessed measurements or plurality of measurements. In some instances, Power Spectrum Density (PSD) can be calculated and the features can be the signal power at a set of discrete frequencies. In some instances, a set of non-PSD related features can be extracted, capturing essentially the same characteristics of the received measurements or plurality of measurements, including, but not limited to: values of real and imaginary parts of Fourier transform, coefficients of wavelet transform or other frequency domain features, and lyapunov coefficients. In some instances, the extracted features can be represented by a set of time-domain features such as length of time intervals between measurements peaks and their statistics such as average, standard deviation, and entropy of distribution. While the measurements or plurality of measurements hardly can be compared to each other due to different length or phase shifts, the features can be compared directly, because after the feature extraction operation, the features extracted from each measurement or plurality of measurements are associated with the same predefined number of feature values. The following operations of logarithmic scaling 5411 and averaging 5412 can be applied to yield the final features. In some instances, these operations are present because logarithmic scaling and averaging are efficient tools to reduce noise and capture biological patterns in the data.

Post-processing operations of dimensionality reduction 5413 can be applied to the extracted features. In some instances, dimensionality reduction operation can be applied in the form of linear detrending or PCA decomposition.

A parallel branch in the same pipeline can perform feature extraction for the downsampled low-resolution component of the received measurements or plurality of measurements. A downsampling operation 5406 can convert the measurements or plurality of measurements into low-resolution measurements which removes information about individual shapes of characteristic motions of a subject and rather approximately captures time evolution of averaged or integrated physical activity along new time sampling intervals 5414 between minutes and days. In some instances, the downsampling can be performed using stride or step counts. In some instances, downsampling can be performed by frequency band filtering and subsequent integration of measurements along new sampling intervals thereby yielding activity counts, for example activity counts per minute. In some instances, downsampling can be performed by calculation of PSD over new sampling intervals and assigning a natural logarithm sum of PSD as a value for the downsampled data points. Other downsampling methods can be used in other embodiments while essentially yielding similar results. In some embodiments, PSD or activity counts are preferable over step counts, because the latter can lose potentially valuable biological information about physical activity other than performing steps during walk, running or other activities.

A feature extraction operation 5415 can perform extraction of numerical or ordinal characteristics of the preprocessed and downsampled measurements or plurality of measurements. In some instances, Power Spectrum Density (PSD) can be calculated and the features can be the signal power at a set of discrete frequencies. In this case, PSD can also be calculated based on the original high-resolution measurements, but is significantly less computationally expensive when performed for downsampled data. In some instances, the downsampled measurements can be quantified, or in other words, binned into bins of different activity levels and then statistics of data point distribution among these levels or statistics of transition rates between these levels is calculated as a feature of the received measurements or plurality of measurements. In some instances, activity transition rates can be calculated between activity states annotated by applying an activity type recognition method to the original high-resolution measurements or plurality of measurements, such as created upon a Human Activity Recognition Dataset [Jorge-Luis Reyes-Ortiz, Luca Oneto, Alessandro Ghio, Albert Samaᵢ, Davide Anguita and Xavier Parra. Human Activity Recognition on Smartphones With Awareness of Basic Activities and Postural Transitions. Artificial Neural Networks and Machine Learning, ICANN 2014. Lecture Notes in Computer Science. Springer. 2014] or the like.

Finally, features of high and low-resolution representations of the received measurements or plurality of measurements can be combined into a single set of feature values which captures the essential biological information that manifested in the measurements or plurality of measurements of physical activity of a Subject. The combined set of features, also referred to as the “Combined Set of Features” 5416 can be used to produce one or more further estimations.

FIG. 5.5 illustrates general aspects of the feature extraction pipeline applied to a measurements or plurality of measurements obtained from a low-resolution sensor. The figure also describes in more detail key aspects of a feature extraction pipeline for the measurements or plurality of measurements obtained from a low-resolution sensor or for downsampled measurements or plurality of measurements as shown in FIG. 5.4. All operations can be grouped into a sequential pipeline of pre-processing 5501, feature extraction 5502, and post-processing 5503. Operations in this pipeline may be run in parallel for extracting different types of features for the same low-resolution measurements or plurality of measurements. The exact order and number of operations can differ between the different embodiments of the invention depending on the estimated value and on the type of sensor from which the measurements or plurality of measurements are obtained.

First, a set of preprocessing operations can be applied 5504 to the measurements or plurality of measurements to determine whether the measurements or plurality of measurements meet the quality requirements. The measurements that meet the quality requirements can then be transformed 5505 into a form appropriate for further feature extraction. The quality checks can be followed by a quality filter 5507 that ensures that a valid estimation can be performed on the measurement or plurality of measurements.

In some embodiments of the invention, the quality checks 5504 can be performed for intervals comprising full days and there can be requirements for threshold cutoffs, for minimal duration of activity period during the day, and for minimal amount of total activity or maximum activity level. In some embodiments, the minimal requirement can be that physical activity is recorded such that the measurements or plurality of measurements cover an interval of fixed duration, for example an interval longer than ten hours either continuously or intermittently. In the case of measurements that cover an interval intermittently, a nearly uniform distribution can be required such that a set of measurements or plurality of measurements can be obtained in each consecutive hour, or two hours, or three hours.

Measurements that do not meet the quality check can be dropped out, disregarded, or otherwise removed. In FIG. 5.5, for example, the measurement input into quality check 5506 does not meet the minimal requirements and is dropped out as indicated by the black diamond. Measurements that meet the quality check can move on to a quality filtering step 5507. As shown, the measurement input into quality check 5504 passed successfully and is moved on to a quality filter 5507, which performs further checking and filtering to determine if the measurements can move on to further estimation procedures. In many instances, at least several days of measurements are required to achieve statistically confident averaging for estimation of age, BMI, type 2 diabetes status, hazard rate or hazard ratio. In some instances, the number of required days is greater if measurements have significant gaps. In some instances, the minimum number of required days is 7 days or 28 days.

One or more transformation operations 5505 can be applied after the quality filter 5407. In some embodiments a logarithmic scaling is applied. In some embodiments, additional operations can be applied including: threshold cutoff clipping, frequency band filtering, averaging or smoothing using a moving window, and logarithm scaling. These operations are useful to treat or remove certain artifacts or outliers that can appear in result of errors or defects during measurement process. For example, if a sensor is known to have a growing or decaying trend and in other aspects provides valid measurements, a frequency band filter can be applied to filter out any frequencies below daily circadian rhythm.

Feature extraction operations 5502 can perform extraction of numerical or ordinal characteristics of the preprocessed measurements or plurality of measurements. In some instances, Power Spectrum Density (PSD) 5508 can be calculated and the features can be the signal power at a set of discrete frequencies. In some instances, other features can be extracted, capturing essentially the same characteristics of the received measurements or plurality of measurements, including for example: values of real and imaginary parts, Fourier transform, coefficients of wavelet transform or other frequency domain features, or lyapunov coefficients. In some instances, the extracted features can be represented by a set of time-domain features such as the length of time intervals between measurements peaks, and statistics such as average, standard deviation, and entropy of distribution.

In some embodiments, feature extraction can be followed by post-processing operations including: logarithmic scaling 5509 and dimensionality reduction 5510. In some instances operations, for example logarithmic scaling, can be used to reduce noise and capture biological patterns in the data. In many instances, a dimensionality reduction operation is applied in the form of linear detrending or PCA decomposition. In some instances, the exact order or the number of the operations are different.

Features of other types can be extracted in parallel for processed or unprocessed measurements. In some instances, a parallel branch can include additional pre-processing operations, and different methods for feature extraction. In some instances, a preprocessing operation can be used to convert the measurements or plurality of measurements to logarithmic scale 5505. In some instances, the measurements or plurality of measurements can be quantified, or in other words, binned into bins of different activity levels of activity and then statistically analyzed based on the datapoint distribution among signal levels 5511, or the statistical analysis of transition rates 5512 between signal levels which can be calculated to yield an activity transition matrix 5513 as a feature of the received measurements or plurality of measurements. In some embodiments, these feature extraction operations can be followed by post-processing operations including: logarithmic scaling 5514, inputing missing values 5515, and dimensionality reduction 5516. In some instances, these operations can be present because logarithmic scaling and inputting missing values provide efficient tools to reduce noise and capture biological patterns in the data. In some instances, an operation for dimensionality reduction can be applied in the form of linear detrending or PCA decomposition. In some instances, the exact order or the number of the operations can be different.

As depicted in FIG. 5.5, different features of the received measurements or plurality of measurements can be combined into a single set of feature values that captures the essential biological information manifested in the measurements or plurality of measurements of physical activity of a subject. The combined set of features, also referred to as the combined set of features 5517 can be used for further estimations.

Implementing Evaluation Pipelines

Once the evaluation pipelines are chosen, the evaluation procedure including the sequence of steps necessary to evaluate the estimations from the measurements or plurality of measurements, can be constructed. The evaluation pipeline describes the mathematical procedures used to evaluate a single estimation from a particular set of sensor data. The evaluation procedure describes the steps to be done during the evaluation of all estimations from any measurements or plurality of measurements.

The evaluation procedure may comprise all the transformations from the evaluation pipelines implemented within the evaluation procedure and the optional pipeline selection procedures. Practical aspects, like reusing intermediate data and sequential or parallel data flows may also be part of the evaluation procedure.

One of the important practical aspects within the evaluation procedure that is not addressed in the evaluation pipeline is data accumulation. Some nodes within the evaluation pipeline, for example a node that calculates the Fourier transform, requires a number of data points to be present at once and therefore some accumulation of sequentially delivered data before this node can proceed can be necessary. Other nodes, for example a node that calculates pointwise functional transformation (e.g. calculation exponent or logarithm), can process values one by one and do not need to accumulate. Other nodes, for example a node that calculates the average value, can be implemented in such way that their internal state is updated immediately upon receiving data and thus data accumulation is not necessary even though the node cannot produce an output until the full slice of data is obtained. The data accumulation and the memory buffers needed for said accumulation are implementation details and thus are not described as part of the evaluation pipeline. In some embodiments, the accumulation can be performed on a larger scale than what is absolutely necessary due to a variety of reasons such as: simplifying implementation, reducing the power consumption of the apparatus, and reducing the cost of the development or the cost of the apparatus by using readily available components.

Whenever data accumulation is necessary or preferable, the type of buffers required can be determined by a person of ordinary skill in the art upon implementing a selected set of evaluation pipelines chosen specifically for the apparatus. The particularities of the evaluation procedure include but are not limited to: data reusing, sequential or parallel data flows, and sequential or parallel nodes execution. Different evaluation procedures implemented within different apparatus are exemplified with particularities as described herein.

Preferable Evaluation Pipelines

In a preferable embodiment, the evaluation pipeline utilizes data from one or more low-resolution and one or more high-resolution sensors to produce an estimation.

In some embodiments of the invention, the evaluation pipeline utilizes data from one or more high-resolution sensors. In these instances, the measured intervals from one or more of the sensors are long enough to satisfy the requirements for the low-resolution sensors. Data from high-resolution sensors can be downsampled, averaged, or filtered to produce data that resembles low-resolution sensors and therefore the embodiments describing applications to high-resolution sensors can effectively be implemented in either preferred mode of the invention and to high-resolution or low-resolution sensors.

In some embodiments of the invention, the evaluation pipeline utilizes only data from the one or more high-resolution sensors and none of the sensors can be considered a low-resolution sensor. Such embodiments are viable, as exemplified later herein, and are within the scope of the invention. In some embodiments of the invention, the evaluation pipeline utilizes only data from the one or more low-resolution sensors.

The accuracy of the evaluated age may correspond to a Pearson correlation of about 0.55 or higher. The accuracy of the evaluated age may correspond to a Pearson correlation of about 0.65 or higher. The accuracy of the evaluated age may correspond to a Pearson correlation of about 0.7 or higher. The Pearson correlation may be within a range from about 0.55 to about 0.75. The Pearson correlation may be within a range from about 0.65 to about 0.85. The Pearson correlation may be within a range from about 0.7 to about 0.9. The Pearson correlation may be The actual age for a group of subjects with a uniform distribution of actual age may be in a range from about 20 to about 70 years old. The actual age for a group of subjects with a uniform distribution of actual age may be in a range from about 40 to about 70 years old. The subject may be a member of a group of subjects. The subject may not be a member of a group of subjects.

The evaluated aged of the subject may be classified among a plurality of classes. The plurality of classes may be selected from the group consisting of young, old, adult, or the like.

The accuracy of the evaluated diabetes type 2 status may correspond to a sensitivity and selectivity. The sensitivity may be at least about 0.6, for example within a range of 0.6 to about 0.9. The sensitivity may be at least about 0.75, for example within a range from about 0.75 to about 0.95. The selectivity may be at least about 0.8, for example within a range of about 0.8 to about 0.95. The selectivity may be at least about 0.75, for example within a range from about 0.75 to about 0.95. The subject may be a member of a group of subjects. The subject may not be a member of a group of subjects.

The evaluated diabetes type 2 status of the subject may be classified among a plurality of classes. The plurality of classes may be selected from the group consisting of normal, borderline, diabetic, or the like.

In some embodiments, the feature may be associated with an age of the subject. The diabetes type 2 status of the subject may be evaluated in response to the evaluated age of the subjected. Alternatively or in combination, the diabetes type 2 status of the subject may be evaluated in response to a body mass index of the subject. The body mass index may comprise the evaluated body mass index wellness parameter or a body mass index input from another source.

The accuracy of the evaluated hazard rate or hazard ratio may be greater than an ROC AUC of about 0.6. The ROC AUC may be within a range from about 0.6 to about 0.9. The accuracy of the evaluated hazard rate or hazard ratio may be greater than a concordance index of about 0.6. The concordance index may be within a range from about 0.6 to about 0.9. The accuracy may be determined for a group of subjects for which the ROC AUC is determined. The subject may be a member of a group of subjects. The subject may not be a member of a group of subjects.

The hazard ratio may comprise a ratio of hazard rates between the subject and a reference hazard rate. The reference hazard rate may comprise an average hazard rate of a reference population. The hazard rate may be evaluated in response to the evaluated hazard ratio combined with a reference hazard rate. The hazard ratio may be evaluated in response to the evaluated hazard rate combined with a reference hazard rate. The evaluated hazard rate or hazard ratio may comprise a hazard rate or hazard ratio for 5-year follow up.

In some embodiments, evaluating the hazard rate may comprise evaluating an age-dependent component, an age-independent component, or a combination thereof. Evaluating the age-independent hazard rate component may comprise evaluating an age-detrended hazard rate of the subject. In some embodiments, evaluating the hazard ratio may comprise evaluating an age-dependent component, an age-independent component, or a combination thereof. Evaluating the age-independent hazard ratio component may comprise evaluating an age-detrended hazard ratio of the subject. The hazard rate or hazard ratio may be evaluated according to a Cox proportional hazards model, an accelerated failure time model, or optimization parameters of a Gompertz-Makeham law of mortality, or a combination thereof.

The evaluated hazard rate or hazard ratio may be classified among a plurality of classes. The plurality of classes may be selected from the group consisting of low, neutral, high, or the like.

The evaluated life expectancy of the subject may be classified among a plurality of classes. The plurality of classes may be selected from the group consisting of short, normal, long, or the like.

The evaluated body mass index of the subject may be classified among a plurality of classes. The plurality of classes may be selected from the group consisting of slim, normal, overweight, or the like.

In some embodiments, a pregnancy status of the subject may be evaluated in response to changes in the body mass index of the subject. The change in the body mass index may comprise a change from a first body mass index to a second body mass index greater than the first body mass index.

In some embodiments, the wellness parameter may be evaluated exclusively in response to a combination selected from the group consisting of an input gender of the subject, the feature and the plurality of features extracted from the plurality of measurements obtained by the sensor coupled to the subject. The wellness parameter may optionally be evaluated exclusively in response to a combination selected from the group consisting of the feature and the plurality of features extracted from the plurality of measurements obtained by sensor coupled to the subject.

### EXPERIMENTAL

The following examples provided in accordance with the present disclosure, and a person of ordinary skill in the art of data science can understand the terms used in these examples.

The examples below describe similar set of features, also referred to as descriptors in data sciences field, which are calculated as follows, with particular values of parameters provided in each example individually:

Power Spectral Density (PSD) (of time series)—frequency dependent function describes the distribution of power into frequency components composing that signal. PSD is calculated using Welch's method [P. Welch, “The use of the fast Fourier transform for the estimation of power spectra: A method based on time averaging over short, modified periodograms”, IEEE Trans. Audio Electroacoust. vol. 15, pp. 70-73, 1967.] with Hann window function [Harris, F. J. (1978). “On the use of windows for harmonic analysis with the discrete Fourier transform”. Proceedings or the IEEE. 66: 51] with window length n.

Bins—set of K disjoint intervals [bᵢ₋₁, bᵢ), i=0 . . . K that cover all time series values. In some embodiments b₀=−∞ and b_{K}=+∞, so all possible values are covered.

Transition matrix (of time series x=[x₁, x₂, . . . ])—square matrix Wᵢⱼ with size K×K, where K is the number of given bins. Transition matrix is calculated as follows. First, time series x is converted to time series y=[y₁, y₂, . . . ], where y is the number of bin which contains xₖ. Then each cell Wᵢⱼ is calculated as a number of n such that yₙ=j and yₙ₋₁=i. Finally each cell Wᵢⱼ in divided by sum Wᵢⱼ over and then sum Wᵢⱼ over j is subtracted from each diagonal cell Wᵢᵢ.

Activity histogram (of time series)—K-dimensional vector of values in range [0, 1], where K is the number of given bins. Each vector value aᵢ=Nᵢ/N, where N is the number of values in the time series and Nᵢ is the number of values in the time series which are within i-th bin. Activity histogram can be calculated for all time series or for a week (weekly activity histogram) and for one day (daily activity histogram) and alike.

### Example 1

Example 1 illustrates that features computed for measured samples of 50 Hz from an accelerometer tracking human activity can be used to evaluate age with supervised machine learning approach to yield a predictive model. The example further illustrates how the features resulting from the trained data model, can be used on new samples of 50 Hz accelerometer track of human activity, to predict the age of the subject using the previously generated predictive models.

Receiving Sample of Subject Activity

Approximately 75,000 samples of individual acceleration measurements were obtained from UK BioBank, where each acceleration measurement was obtained from an Axivity AX3 tri-axial accelerometer, continuously measured at 100 Hz during 7 days. Acceleration was measured in a free-living setup, without requiring the subject to perform any specific activities. The subjects were allowed to engage in freely living activity with these externally worn sensors. The measurements were stored at the Axivity memory and then downloaded to a PC using the Axivity software program. The sample of the plurality of measurements was submitted to the feature extraction and age evaluation pipeline comprising a combination of the operations depicted in FIG. 5.4, which describe this process in detail.

Computing Sample Features (Preprocessing, Feature Extraction, Postprocessing)

Preprocessing:

A preprocessing procedure was applied to a received sample of the plurality of measurements. First, a quality filter was applied to ensure that the received sample had a “longest resting period per day” that did not exceed than 10 hours. If not, the sample would have been rejected and no evaluation would have been performed for the sample. The sample of the plurality of acceleration measurements comprised three parallel time series of acceleration along each spatial axis, and were transformed to the absolute value of acceleration vector at 100 Hz. The time series of the absolute value of acceleration vector was then downsampled to 50 Hz by retaining every second measurement. The result comprised a series of non-negative acceleration values that were then sliced into a time ordered set of consecutive time series of one minute length each.

Feature Extraction:

PSD was calculated for two frequency ranges (low and high) separately to divide features into two groups. PSD for the high frequency range 0.05-25 Hz was calculated directly for each one-minute slice of 50 Hz time series of absolute value of acceleration vector by Welch method with window length of 1024 data points yielding a set of 513 spectral power values for discrete frequencies of 0.05 Hz to 25 Hz for each one-minute slice.

Additionally, the PSD calculated for each one-minute slice was used to downsample the original time series of acceleration (e.g. plurality of measurements). Specifically, the PSD values for each one-minute slice were summed and converted to a natural logarithm, resulting in a downsampled time series with a 1 minute time interval. Next, a transition matrix with 10 bins with equal lengths, fully covering interval [−15, 5] was calculated for time series with 1 minute time interval.

Postprocessing:

High frequency PSD of sample were further detrended to logarithm of sum of PSD. To do that logarithm of sum of PSD was calculated for each one-minute slice for each sample from dataset. A linear regression model was then built for each of 512 value of PSD. Residuals of linear regression were then averaged within each sample yielding 512 values per sample, referred to as dPSD.

The high-frequency dPSD and transition matrix were merged into a combined set of features for each sample. Specifically, the 512 dPSD values were combined with 100 elements of the transition matrix and normalized to zero mean and unit variance to yield 612 feature values for the received sample of plurality of measurements.

Evaluating the Age of the Subject Based on the Plurality of Features

In this particular example, the age of the subject was evaluated by deep neural networking (DNN). The parameters of the DNN were optimized according to the scheme outlined in FIG. 5.3. The dataset was annotated with the chronological age of the users, and was split into a training set (50,000 samples) and test set (20,000 samples). The DNN architecture was set up with Rectified Liner Units (“ReLU”)—nonlinear activation functions capable of capturing complex interactions between features and nonlinear dependency of the target variable (e.g. age) on the features. The parameters of the neural network were fit using an Adagrad algorithm [Duchi, J. Hazan, E., & Singer, Y. (2011). Adaptive subgradient methods for online learning and stochastic optimization. Journal of Machine Learning Research, 12(July), 2121-2159] with L1 regularization, with dropout equal to 0.25 and the weights and biases of neurons initialized uniformly.

Compared to a linear regression model, the DNN model yielded similar correlation of predicted age to original age=0.8, but significantly improved the reproducibility of distribution of predicted age across subjects. The rest 20% (approximately 20,000 samples) of the dataset was used as a test set to test new samples, and the subject's age was evaluated based on descriptors computed for the test set samples. The evaluated age accuracy was measured by a correlation coefficient of predicted age to original age=0.7. The standard deviation of predicted age from original age from the UK biobank data was 6 years.

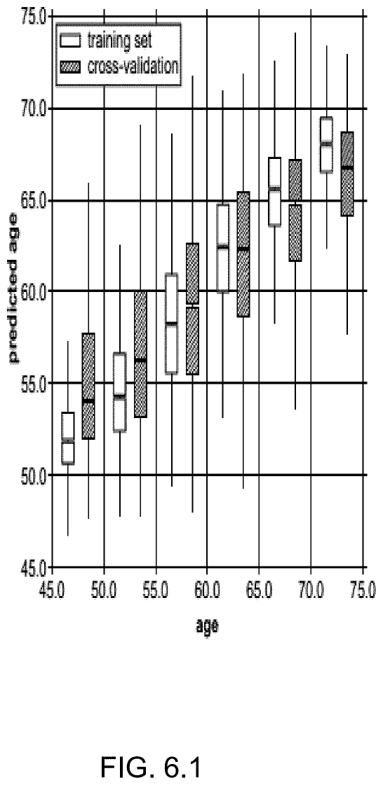

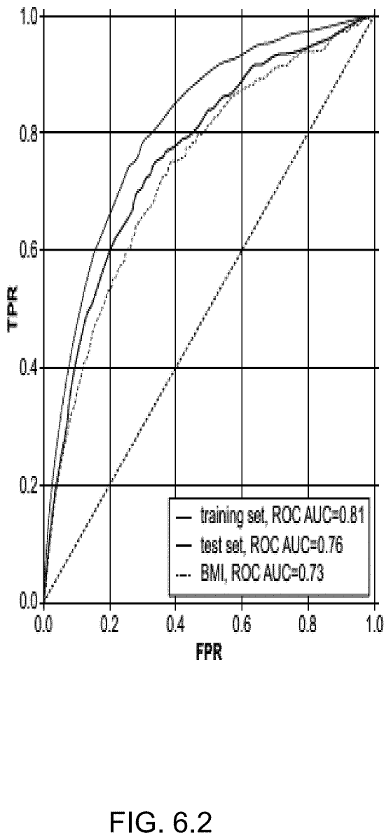

FIG. 6.1 shows the results for the training and test data sets. The evaluation achieved age evaluating accuracy level measured by Pearson's correlation of 0.8 for the training dataset and 0.7 for the test dataset corresponding to root mean squared error of 6 years. After the neural network for evaluation of the age of the subject had been trained and tested it was stored in the form of evaluation procedures, or instructions, and parameters to be further applied in the device, apparatus, or system for evaluation of a subject's Age for new received samples according to the evaluation pipeline comprising feature extraction and Age evaluation procedures as outlined in the dashed box labeled 5302 in FIG. 5.3.

Many modifications can be made to this example yielding the same or similar results and thus remaining within the scope of the present invention. Thus, in some instances the measurements or plurality of measurements can be sampled at different frequencies: 1 Hz, 10 Hz, 200 Hz or higher or lower or any value in between, preferably between 10 to 100 Hz to keep reasonable balance between accuracy and computational costs. In some instances the measurements or plurality of measurements of freely moving physical activity of the subject comprise acceleration measured along only one spatial axis of the sensor device, or gyroscope measurement, or magnetometer sensor measurement. The measurement can either be submitted to the evaluating pipeline built specially for the certain type of sensor, or it can be linearly or logarithmically scaled to the range of acceleration so that the pipeline built for the acceleration measurements is applicable directly. Furthermore, it should be noted that performance for this method may not change significantly if the measurements or plurality of measurements are sliced into slices of different length, including for example, 30 seconds, 2 minutes, 5 minutes, 10 minutes 15 minutes, 30 minutes, 1 hour or any value between or alike. Also, in some instances the number of bins can be varied to, for example: 2, 5, 20, or any value between or alike, preferably between 5 and 10. In some instances, the bin edges are set nonuniformly based on the distribution of occupancy of activity levels in a selection of samples. In some instances the Welch window can be of a different shape or length, for example, including but not limited to: 128, 256, 512, 1024, 2048, 4096, or other values, preferably a power of 2 and selected to keep reasonable balance between evaluation accuracy and computational cost.

Different neural network architectures, parameters, training algorithms, and dataset splits can be used, and can yield results with similar accuracy. In this example, the number of neurons in the input layer was set to the number of features and the number of neurons in the output layer was set to single neuron with linear activation. Additionally, the specific number of hidden layers can be different. In most instances, at least one hidden layer can exist with a reasonably large number of neurons, but there can be any number of layers. Generally, the more the number of hidden layers the more the number of neurons, and the more layers and neurons the more accurate the resulting evaluation pipeline is likely to be.

### Example 2

Example 2 illustrates that features computed for samples from a 50 Hz accelerometer measurement of human activity can be associated with type 2 diabetes status when predictions are made using a predictive model generated through a supervised machine learning approach. This example further illustrates how the features computed for new samples of a 50 Hz accelerometer track of human activity can be associated with type 2 diabetes status through the predictive model to yield an evaluation of type 2 diabetes status. In this example, the dataset and computed features were exactly the same as described in the pipeline of Example 1.

Associating Sample Spectral Features with Subject's Diabetes Status Through a Supervised Learning Approach.

Between about 75-80% (60,000) of the dataset was used to generate the predictive model parameters. About 2-5% of the dataset comprised subjects with positively diagnosed diabetes status (predominantly diabetes melitus type 2), which results in a highly imbalanced dataset (<3000 of 75,000 subjects were diagnosed to be diabetic). Ensembling methods were employed to leverage the huge amount of data from healthy participants. Ensembling was designed such that an equal number of diabetic and healthy subjects were sampled randomly, multiple times, for updating the neural network (NN) model. The NN architecture comprised a sequence of input, output, and 4 hidden ReLU layers with dropout (0.25) and regularization (L2). Models were initialized randomly using He initialization [K. He, X. Zhang, S. Ren, and J. Sun, “Delving Deep into Rectifiers: Surpassing Human-Level Performance on ImageNet Classification,” arXiv:1502.01852 [cs], February 2015]. Only models with ROC AUC greater than 0.7 on global training set were kept. The resulting 62 models were used for prediction with a simple voting scheme (at least 16 models predicting diabetes were required to assign diabetes status to the subject). Shallower NN architecture resulted in lower performance, while deeper architecture resulted in model overfitting. Ensemble of neural networks yielded model with accuracy measured by ROC AUC=0.81.

Associating sample spectral features with a subject's diabetes status through an obtained predictive model

The remaining 20-25% (20000) of the dataset was used as a test dataset, and the subject's diabetes status was evaluated based on features computed for these samples. The evaluated diabetes status accuracy was measured by ROC AUC=0.76, as shown in FIG. 6.2. The optimal threshold can be selected based on optimization of sensitivity and selectivity, as understood by a person of ordinary skill in the art. The present inventors selected a threshold that yielded a sensitivity (true positive rate) 0.6 and selectivity (true negative rate) 0.8.

This procedure demonstrates that the predictive model as described and constructed, can be used to evaluate type 2 diabetes status of new subjects with reasonably good recall and precision.

### Example 3

This example illustrates a method to evaluate age of a subject based on measurements of freely moving physical activity of the subject obtained from step counter sensor.

Receiving Sample of Subject Activity

In this particular example the plurality of measurements were obtained from from step counter sensor of fitness-tracker Fitbit, worn in hip or on belt. Models of Fitbit, used in this example were Fitbit Tracker, Fitbit Ultra, Fitbit One, Fitbit Zip and Fitbit Flex. Described tracker was worn subject during at least 28 days with possible breaks in the data collection. The rage of chronological age of subjects in this example was from 25 to 85 years old.

Fitbit trackers continuously measured number of steps per minute based on three-dimensional accelerometer. The said sample of plurality of measurements was further submitted to the feature extraction and age evaluation pipeline comprising the combination of operations depicted in FIG. 5.5 and described with particular details in the present example below.

Extracting Features (Preprocessing, Feature Extraction, Postprocessing)

Preprocessing:

Upon receiving the said sample of plurality of measurements, the method performed the following preprocessing procedure. First, a quality filter was applied to ensure that received sample has at least 21 day with at least 1000 total steps and 200 minutes with non-zero step numbers per day. If not, the sample would have been rejected and no evaluation can be performed for this sample. If sample passed the described quality filter then 21 last days with at least 1000 total steps and 200 minutes with non-zero step numbers per day would be selected for further analysis.

Feature Extraction:

The feature extraction procedure comprised calculation of six numerical characteristics (features) of the preprocessed sample:

1. PSD calculated for each day with window length 512 min, yielding 257 PSD values.

After calculation PSD were averaged over 21 days and then logarithmically scaled.

2, Transition matrix calculated over all time series with 7 bins with equal lengths, fully covering interval [0, 200].

3, Transition matrix calculated over first 8 hours from each day with the same bins as in pt.2.

4. Transition matrix calculated over last 8 hours from each day with the same bins as in pt.2.

5. Activity histogram with 20 bins with equal lengths, fully cover interval [0, 200].

6. Standard deviation of components of a daily activity histogram, calculated over 21 days with the same bins as in pt.5.

Postprocessing:

In this particular example PSD after initial treatment described above was multiplied by four vectors due to dimension reduction resulting in four values which was used then as features.

Evaluating Age of the Subject Based on the Plurality of Features.

Upon the sample of the plurality of measurements of freely moving physical activity of the subject was received and the sample features were extracted, the said features were submitted to the evaluation pipeline based on extra-trees estimator realisation of “random forest” algorithm [P. Geurts, D. Ernst., and L. Wehenkel, “Extremely randomized trees”, Machine Learning, 63(1), 3-42, 2006.] to yield an evaluated value of age of the subject.

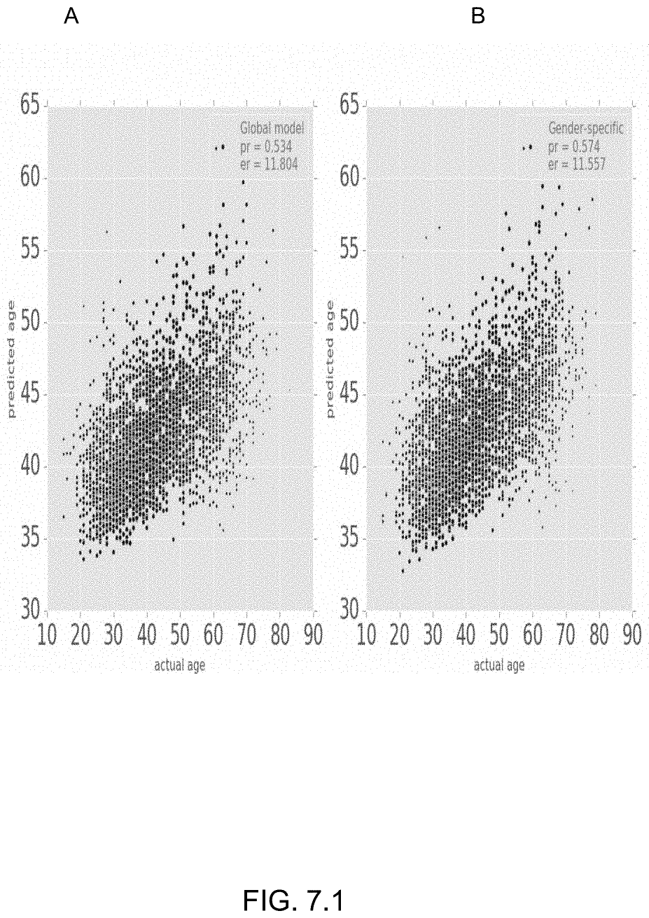

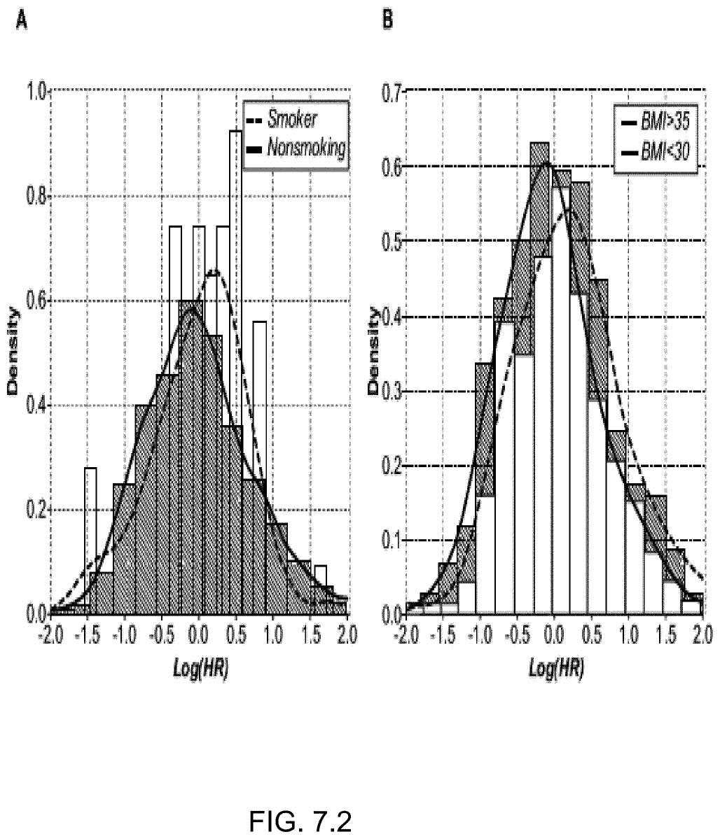

The vectors for PSD dimension reduction and the parameters of extra-trees estimator was obtained at the plurality of measurements of physical activity in the form of 21 daily step counts measurements for 2,400 subjects wearing Fitbit tracker and annotated by their chronological age. The vectors for PSD dimension reduction was obtained by Principal Component Analysis procedure as four main vectors corresponding to four largest singular values. The parameters of the extra-trees estimator was optimized according to the scheme outlined in FIG. 5.3. The plurality of measurements was split into training set, cross-validation set and test set in proportions 5:1:3. The parameters of extra-trees estimator were optimized to 70 trees in the random forest, maximum depth in the tree=5, achieving age evaluating accuracy level measured by Pearson's correlation 0.5 for the test dataset corresponding to root mean squared error of 12 years as shown in FIG. 7.1 (A). Additionally, a gender-specific variant of age-evaluation pipeline was optimized to parameters n_estimators=70, max_depth=5, achieving age evaluating accuracy level measured by Pearson's correlation 0.55 for the test dataset corresponding to root mean squared error of 11.5 years for males as shown in FIG. 7.1 (B). After the extra-trees estimator for evaluation of the age of the subject has been once trained and tested it has been stored in the form of instructions and parameters and can be further applied in apparatus for evaluation of subject Age for new received samples of step counts according to the evaluation pipeline comprising feature extraction and Age evaluation procedures as outlined in the bottom part of scheme in FIG. 5.3.

### Example 4

This example illustrates the method to evaluate hazard ratio of a human subject based on measurements of freely moving physical activity of the subject obtained by a step counter sensor.

Receiving Sample of Subject Activity

In this particular example, the sensor coupled to the subject was ActiGraph AM-7164 (formerly known as CSA/MTI AM-7164, http://actigraphcorp.com). The sensor was placed on an elastic fabric belt, custom-fitted for each subject, and worn on the right hip. Subjects worn sensor during 7 days and removed it before swimming or bathing and at bedtime. In this particular example the plurality of measurements was a continuous time series of stepcounts with one minute sampling rate. The measurements were stored at the ActiGraph memory and then downloaded to a PC using ActiGraph software program.

Extracting Features (Preprocessing, Feature Extraction, Postprocessing)

Preprocessing:

Upon receiving the said sample of plurality of measurements, the method performed the following preprocessing procedure. First, the method tested whether the received sample contain only stepcounts value less or equal to 200. If not the sample would have been rejected and no evaluation can be performed for this sample.

Feature Extraction:

The feature extraction procedure comprises calculation of six numerical characteristics (features) of the preprocessed sample:

1. PSD calculated for each day with window length 512 min, yielding 257 PSD values. After calculation PSD were averaged over 7 days and then logarithmically scaled.

2. Transition matrix calculated over all time series with 7 bins with equal lengths, fully covering interval [0, 200].

3. Transition matrix calculated over first 8 hours from each day with the same bins as in pt.2.

4. Transition matrix calculated over last 8 hours from each day with the same bins as in pt.2.

5. Activity histogram with 20 bins with equal lengths, fully cover interval [0, 200].

6. Standard deviation of components of a daily activity histogram, calculated over 7 days with the same bins as in pt.5.

Postprocessing: After feature extraction each calculated feature was presented as a vector. Then each vector was multiplied by four vectors, predefined for this feature type. This action was performed for dimension reduction and result in 4-dimension vector for each feature. In further analysis both raw features and features after dimension reduction were used.

Evaluating Hazard Ratio of the Subject Based of Plurality of Features

Upon the sample of plurality of measurements of freely moving physical activity of the subject was received and the sample features were extracted and preprocessed, the said features were submitted to the evaluation pipeline. Evaluation pipeline consisted of multiplying the vector of features by the predefined vector \vec{β}, then exponent was calculated from the result, yielding predicted Hazard Ratio for the subject.

In this particular example NHANES 2005-2006 dataset was used for building and testing evaluation pipeline. Sample of data from said dataset consisted of freely moving physical activity (described above) and metadata: chronological age, body mass index (BMI), smoking status (number of cigarettes, smoked per day) and time to death or time to last follow up. From said dataset were selected samples with chronological age more than 40. Then these samples were subjected to preprocessing and feature extraction procedures described above. After that each feature type was subjected to Principal Component Analysis after that four principal vectors corresponding to four largest singular values was chosen as vectors for dimension reduction for postprocessing stage. Then vectors \vec{β} for prediction of Hazard Ratio of the subject was found by the Cox Proportion Hazard model [Cox, David R (1972). “Regression Models and Life-Tables”. Journal of the Royal Statistical Society, Series B. 34 (2): 187-220.] for features after dimension reduction or by the 12-regularized Cox Proportion Hazard model [Friedman, Jerome, Trevor Hastie, and Rob Tibshirani. “glmnet: Lasso and elastic-net regularized generalized linear models,” R package version 1 (2009).] for raw features. During training the model samples of said dataset was splitted into train and test subsets in proportion 2:1, using first one for training the model and second for test of the trained model. ROC AUC value was used to estimate model accuracy for scoring of mortality event by Hazard Ratio of the subject, in this example ROC AUC was 0.67 for Cox model and 0.69 for regularized Cox model. Also interpretability of trained model was checked by association of value of natural logarithm of Hazard Ratio with healthy and unhealthy lifestyles of human subject. To do that samples with predicted Hazard Ratio were split into two groups with healthy and unhealthy lifestyles. Said splitting was done for smoker status (nonsmoking, smoker) and obesity status (BM/<30, BMI>35). As one can see from FIG. 7.2 (for regularized Cox model), human subjects with healthy lifestyle (nonsmoking or BMI<30) had less average log Hazard Ratio than human subjects with unhealthy lifestyles (smoker or BMI>35).

### Example 5

This example illustrates the method to evaluate age, hazard ratio and life expectancy of a subject based on measurements of freely moving physical activity of the subject obtained from an accelerometer sensor.

Receiving Sample of Subject Activity

In this particular example, the sensor coupled to a subject was ActiGraph AM-7164 single-axis piezoelectric accelerometer worn on hip for 7 days. The plurality of measurements obtained by sensor was received in the form of a continuous time series of activity counts per minute calculated according to ActiGraph algorithm. Activity counts represent sum of acceleration measurements corresponding to each one minute interval were calculated by Actigraph software program upon downloading acceleration measurements from the device memory to PC.

Additional sensor coupled to a subject of the present example was Axivity AX3 tri-axial accelerometer worn on wrist for 7 days. The plurality of measurements obtained by sensor was received in the form of a continuous time series of acceleration along each of the three accelerometer axes sampled at 100 Hz rate. The measurements were stored at the Axivity memory and then downloaded to a PC using Axivity software program.

The resulting sample of plurality of measurements was further submitted to the pipeline of the disclosed method for evaluation of age and hazard ratio, (and further evaluation of derived parameter—life expectancy in response to evaluated hazard ratio) comprising the combination of operations depicted in FIG. 5.5 and described with particular details in the present example below.

Extracting Features (Preprocessing, Feature Extraction, Postprocessing)

Preprocessing:

Upon receiving the said sample of plurality of measurements, the method performed the preprocessing procedure to ensure that the received sample meets the minimal quality requirements to be eligible for evaluating of age and hazard ratio.

First, if the received plurality of measurements was obtained by ActiGraph sensor, the measurements would have been converted to binarized values as follows. The activity count was incremented by a unit value and converted to natural logarithm scale. If the received plurality of measurements was obtained by 100 Hz Axivity accelerometer, the acceleration measurements would have been converted to binarized activity counts per minute as follows. The absolute value of acceleration is calculated based on acceleration measurements along three individual accelerometer axes. The entire plurality of measurements was split into consecutive slice of 6000 measurements each, corresponding to 1 minute periods. Each 1 minute period was then assigned a numerical value of logarithm of sum of PSD with window length 1024.

Next, a quality filter was applied to ensure that the sample of plurality of measurements comprises seven days with non-zero activity and that the average activity level is not an outlier, i. e. is inside the range between 50.0 and 5000.0 activity counts per minute. A day was considered to have zero activity if it comprised less than one hour of non-zero activity counts recorded by ActiGraph sensor or less than one hour with absolute value of acceleration different from gravitational acceleration. If the received sample did not meet either requirements on number of non-zero activity days or on non-outlier average level of activity it would have been rejected and no further evaluation of wellness parameters would have been performed.

Feature Extraction:

In this particular example the feature extraction procedure comprised calculation of activity transition matrix as a numerical characteristic of the received and preprocessed sample of plurality of measurements. For this purpose each measurement in the preprocessed data series was assigned to a corresponding activity bin, wherein the number of bins was set to 10 and covered uniformly the range of preprocessed measurement values between 0.0 and 10.0 for ActiGraph sensor. All values greater than 10.0 were assigned to the highest bin. If the received plurality of measurements was obtained by Axivity sensor, the measurements were assigned to 10 bins according predefined bin edges which were stored as the parameters of the present method. The said edge values were obtained using quantile normalization procedure on the training set to match Axivity sensor measurements onto the scale of ActiGraph sensor. The quantile normalization was performed using a random subset of 10 000 samples to obtain such bin edges that the occupancy of each bin was the same as in NHANES dataset.

The transition matrix was calculated as a square matrix, wherein each off-diagonal cell of the matrix in row i and column j corresponded to number of transitions from activity bin j to activity bin i between two consecutive time-ordered one-minute slices, and then was divided by the total number of measurements in bin j along the plurality of measurements. The diagonal element in row i was a negative sum of all off-diagonal elements in row i.

Postprocessing:

In this particular example, the extracted 100=10×10 elements of the activity transition matrix were further submitted to the noise-reducing postprocessing procedure, comprising consecutive operations of truncation of the transition matrix, imputation of missing values (initially assigned zero values), conversion to natural logarithm scale and projection onto predefined vectors. The truncation was performed to exclude transitions to the lowest activity bin and highest two activity bins, finally yielding a matrix of 49=7×7 bins. The imputation was done as follows: each element of transition matrix with zero value was replaced with the predefined value stored as the parameter of the present method. The projection onto the predefined vectors was done by flattening of the transition matrix into a vector of ordered values and then retrieving the vectors stored as the parameter of the present method and multiplication of the flattened matrix by these vectors yielding numerical values of the projections. The resulting projection values were further used as the feature values characterizing the properties of the received sample of plurality of measurements.

Evaluating the Age of the Subject Based on the Plurality of Features

In the present example the hazard ratio for a subject was evaluated using Cox proportional hazards model, wherein the parameters of the model were optimized and validated using UK Biobank and NHANES datasets. Optionally an age-dependent component of hazard ratio was evaluated and reported as the evaluated age of the subject. Optionally the age-independent component of hazard ratio was evaluated and used to further evaluate a derived wellness parameter of life expectancy. Optionally, an input information on the gender of the subject was used to invoke the model for evaluation of hazard ratio with higher accuracy.

In this particular example the instructions for extracting features and evaluating of the hazard ratio, age-related component of hazard ratio, age-independent component of hazard ratio, and optionally the derived wellness parameter of life expectancy were implemented in the form of a python script. The method implementation did not require any knowledge of the subject's actual age or any indication of the subject's life expectancy or other metadata, except for an optionally input gender of the subject. The parameters for cutoff threshold values, activity bin edges, weight coefficients and shifts were stored in a parameter file which was loaded by the script for performing the evaluation.

The method implementation in the form of instructions and parameters was created and validated as described below. Optimization of method parameters was based on analysis of the dataset of 14,000 samples of freely moving physical activity from NHANES 2003-2004 and 2005-2006 studies, measured by ActiGraph sensor. Each sample was annotated with the age of a subject and the survival or mortality status of a subject within 1 to 8 years follow up after the measurement of physical activity. It should be noted that after each sample was preprocessed according to the process described in this example above, the samples with unordinary high (more than 5,000.0 activity counts per minute) or unordinary low (less than 50.0 activity counts per minute) average value of nonzero activity counts were rejected, the fraction of rejected samples being less than 5%. The exclusion of the first lowest activity bin was made intentionally to produce a set of features corresponding to walking bouts and resistant to gaps in activity measurements since all transition rates from and to the resting activity state were thus discarded. This procedure ensured that the method would be further applicable to plurality of measurements obtained from a wearable device or a smartphone of a user which is supposed to record only walking or other high-level physical activity of a subject when the subject is likely to have the device with him or her in a freely moving setup.

The parameters for the postprocessing operations were obtained as follows. The predefined parameters for imputation were obtained by calculating an average of nonzero values of each element of the transition matrix of NHANES participants and then were stored as the parameters of the method. The predefined vectors were obtained as the eigenvectors of covariance matrix of all 49=7×7 elements of transition matrix across all samples obtained for NHANES participants of age 30 or older. Only 10 eigenvectors corresponding to the 10 largest eigenvalues were retained. Projection of elements of flattened transition matrices onto the eigenvector corresponding to the largest eigenvalue showed correlation with age of participants of 0.6. Since projections onto other eigenvectors showed no significant correlations with age, the projection onto the eigenvector corresponding to the largest eigenvalue was further used to evaluate the age-related component of hazard ratio.

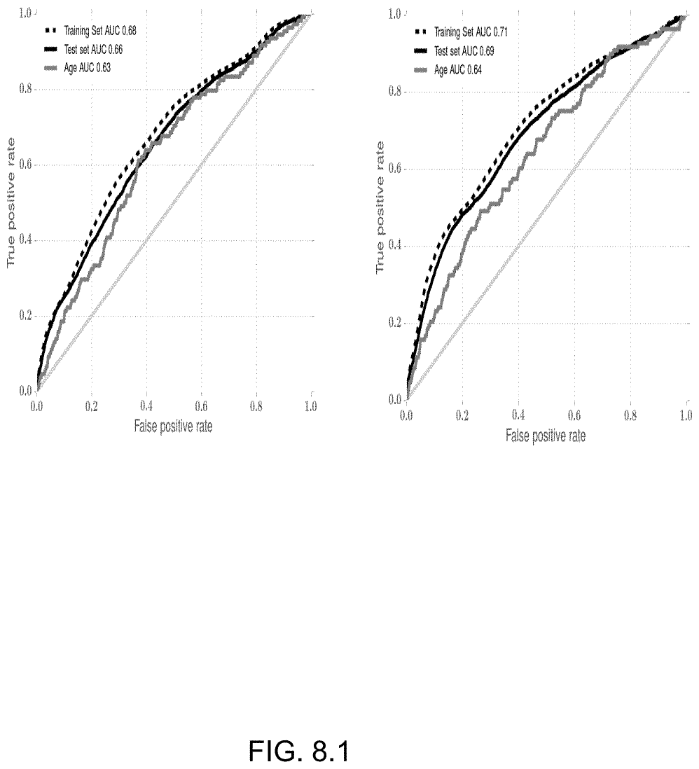

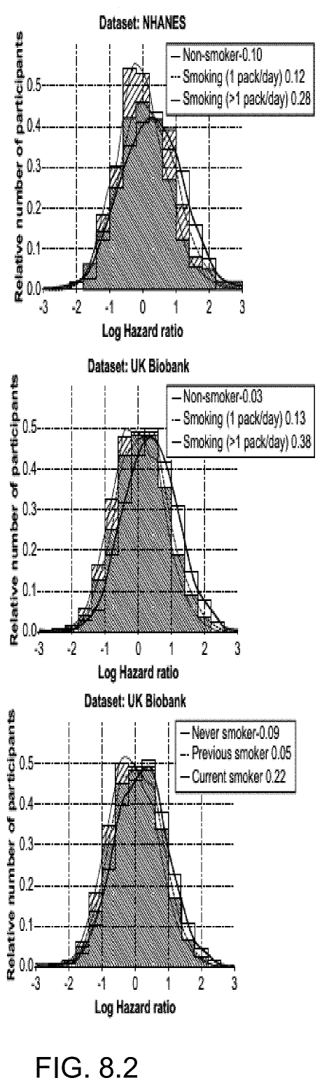

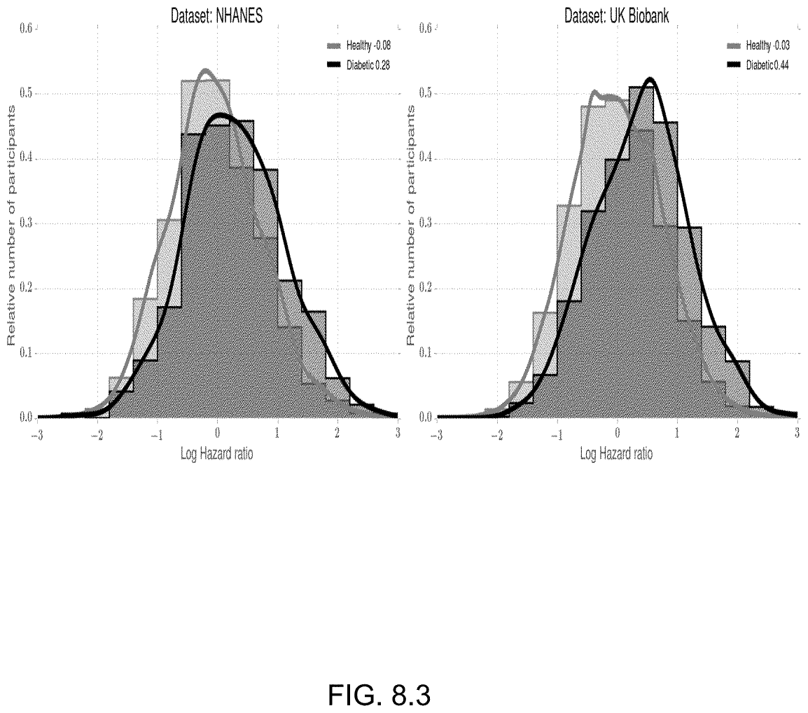

Parameters of the Cox proportional hazard ratio model were optimized using 3,000 samples annotated with survival or mortality status for participants aged 40-70 within 1 to 5 years follow up, of which 100 were reported to die within 5 years follow up. The parameters were tested for absence of overfitting effect by 100 cross validation runs with splitting data into the training and test sets in proportion 4:1. The accuracy of evaluated hazard ratio was assessed in terms of scoring samples corresponding to mortality outcomes depicted as ROC curve and shown in FIG. 8.1 for model based only on features extracted from plurality of measurements of physical activity (left) and for model adjusted for explicitly input gender of a subject (right). In both cases the average ROC AUC reflecting the quality of scoring mortality outcomes was outperforming scoring by chronological age for both the training and cross validation sets.

The evaluated hazard ratio was further tested for evaluation of derived wellness parameters. First, we demonstrated that the evaluated hazard ratio was efficient to score participants according to their smoking status in dose-dependent manner. Thus the highest hazard ratios for those who smoke more than 20 cigarettes per day and the lowest hazard ratios for those who do not smoke, while those who smoke 1-20 cigarettes per day had intermediate distribution of hazard ratio for both NHANES (FIG. 8.2, left pane) and UK Biobank (FIG. 8.2, middle pane) datasets. In addition we demonstrated that the evaluated hazard ratio can be further used to score the subjects according to their current, previous and never smoking status, showing that previous smokers have lower hazard ratios (FIG. 8.2, right pane). Also we demonstrated that the evaluated hazard ratio was efficient to evaluate participants according to their type 2 diabetes status as shown for both NHANES (FIG. 8.3, left pane) and UK Biobank (FIG. 8.3, right pane) datasets. Therefore, the method of the present example can be used to evaluate the smoking status and the type 2 diabetes status as a derived wellness parameter in response to evaluated hazard ratio.

Next, life expectancy was evaluated as a derived wellness parameter in response to evaluated hazard ratio according to Gompertz law of mortality (equation 5.1):

M(t)=M₀ exp(a*t)

Natural logarithm of hazard ratios evaluated according to Cox model were detrended on age yielding age-independent log hazard ratio component Θ which can be interpreted as the ratio of the pre-exponential Gompertz law parameter M₀ between the subject and average population. Life expectancy was thus evaluated as a baselevel life expectancy for average population and adjustment additive term (equation 5.2):

T_{average}=T₀−Θ/a

Where “a” is Gompertz exponential parameter and was assumed to be a=0.085 [1/year] for human subject. Therefore, the evaluated hazard ratios yield an estimation of life expectancy difference between smokers and non-smokers of 5 years, which corresponds to half of the difference of 10 years according to Center for Disease Control report.

All the correlation coefficients, p-values and ROC AUC values reported in this example were the measurements of the accuracy for evaluation of age, survival hazard, life expectancy and related characteristics.

### Example 6 (Smartphone App)

In an exemplary embodiment of a method and system as described herein, the subject may be a user of a smartphone. The smartphone may be externally coupled to the user. For example, the smartphone may be routinely carried in a pocket or in a bag, and may be easily reached by the user. The smartphone may include an accelerometer sensor, and the sensor may output time-ordered plurality of measurements representing the user's physical activity measurements. Free-living physical activity in the present example implies that the user's behavior is uncontrolled and the apparatus is only passively measuring physical activity without requiring the user to perform any special actions. The apparatus may update the evaluated age, type 2 diabetes status and lifestyle-associated hazard ratio upon the user's request, or on a regular basis. The updates may be presented to the user in one or more visual forms, such as on the screen of a smartphone. In other embodiments, evaluated age and lifestyle-associated hazard ratio can be used to compare the physical activity of the subject with population distribution according to environmental and lifestyle factors. The details of the architecture of the apparatus and the operations pipeline are described below.

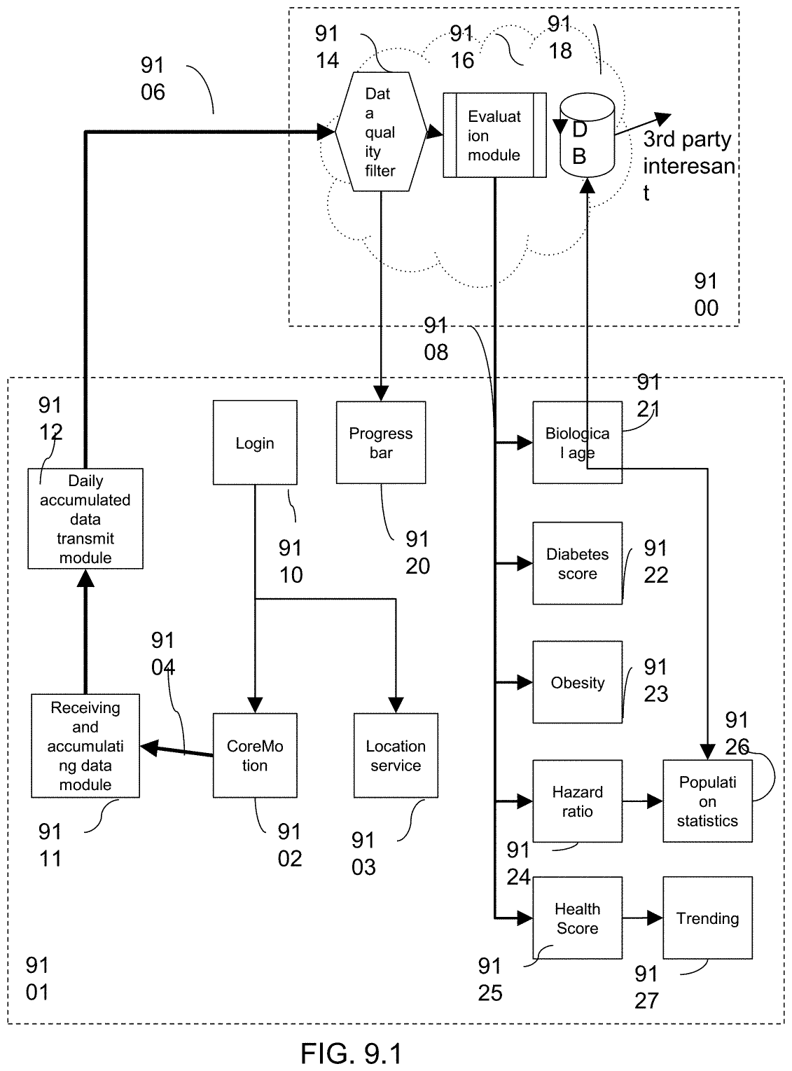

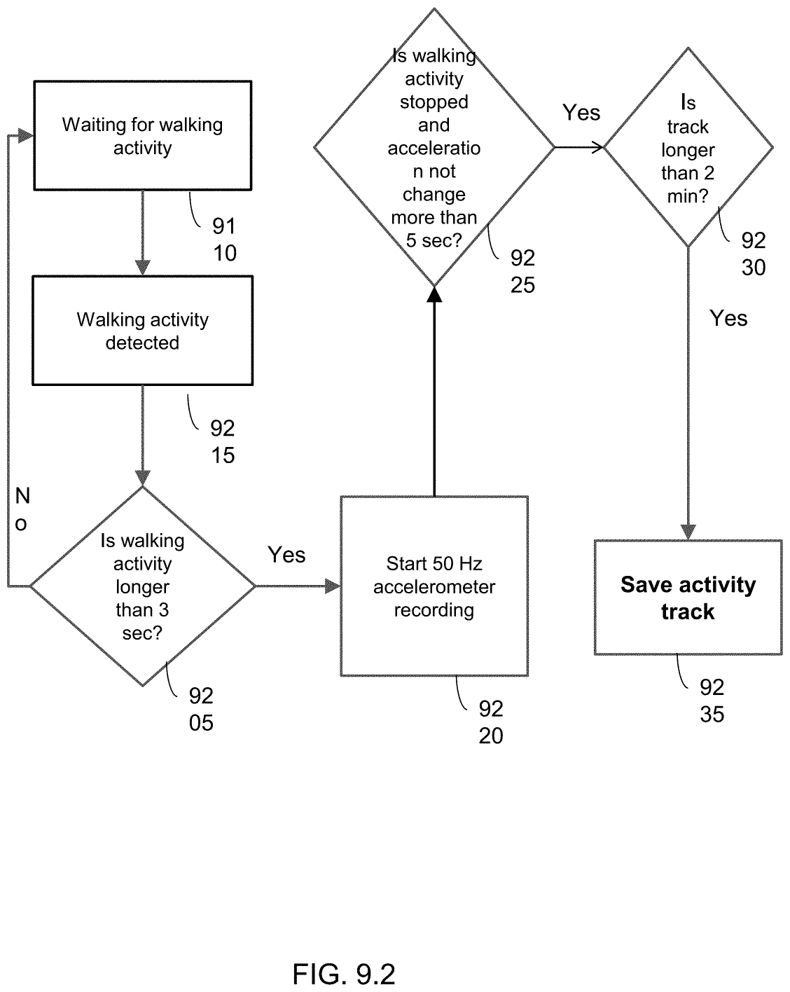

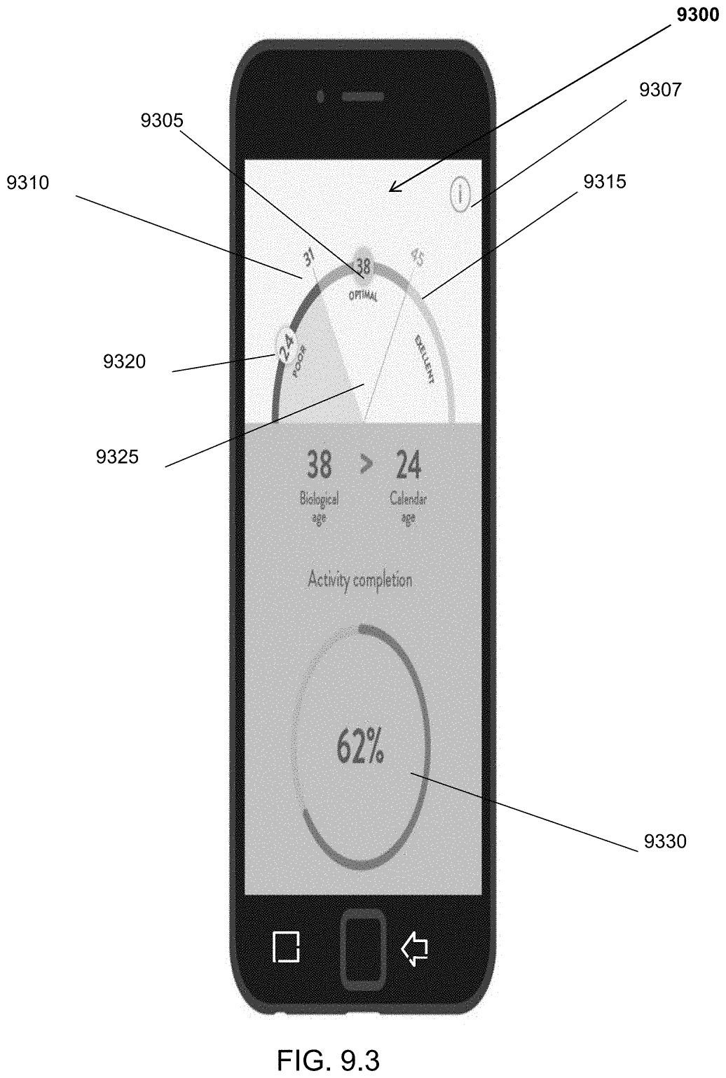

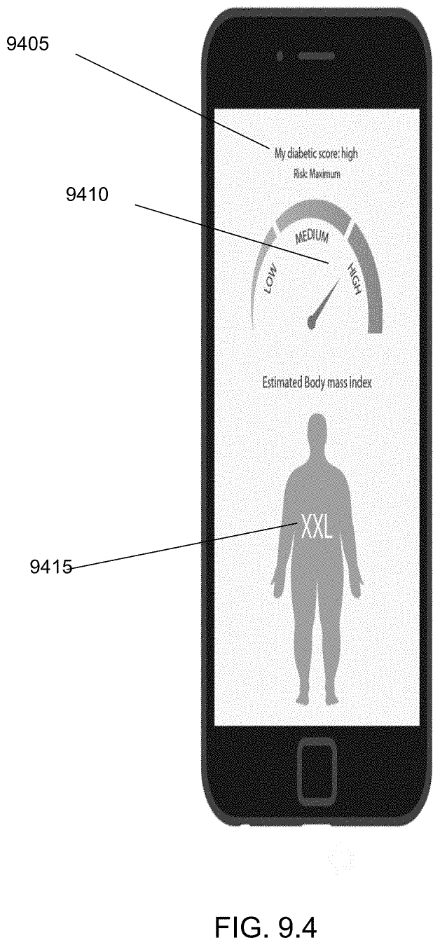

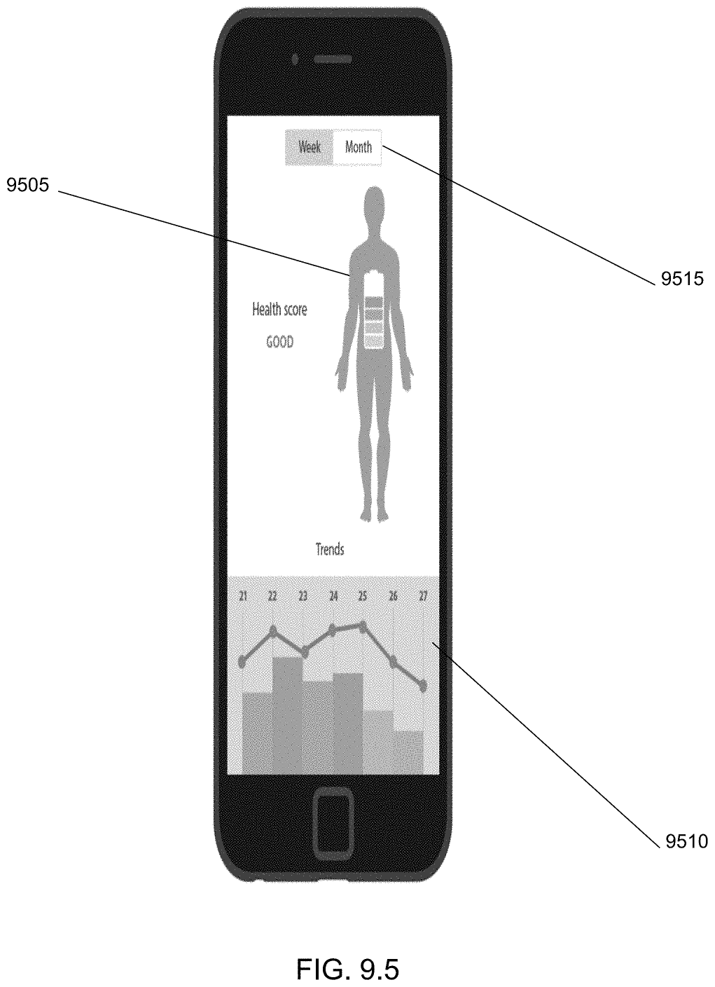

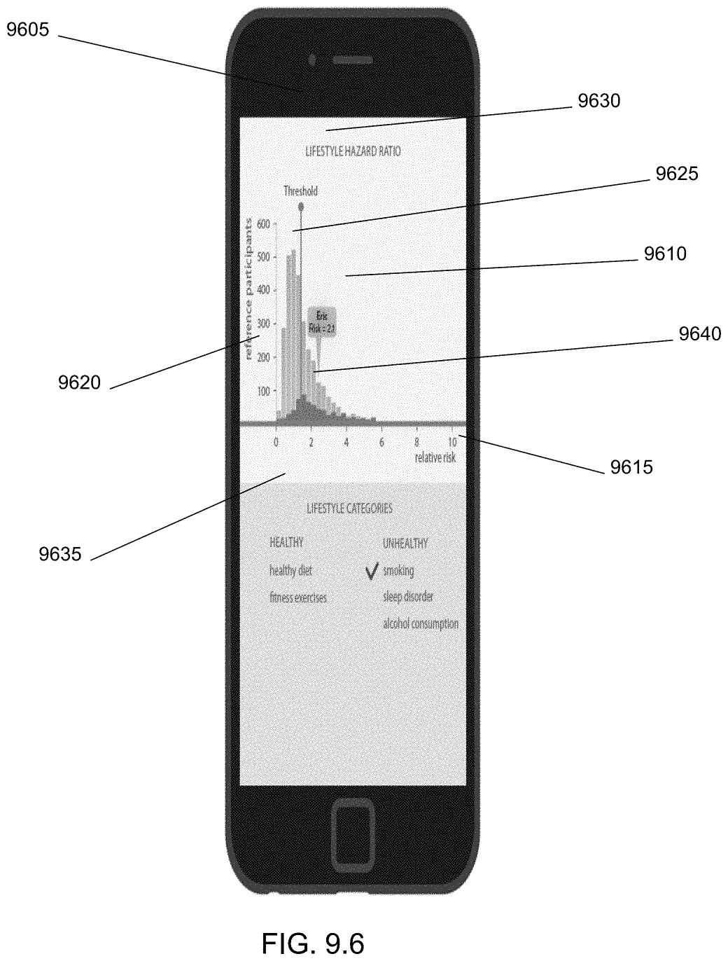

FIG. 9.1 shows the general architecture of an apparatus which comprises a software-implemented system. The system may comprise a back-end module 9100 running on a server, and a front-end module 9101 running on the user's smartphone. The front-end module is interacting with smartphone operating system services 9102, 9103 to receive 9104 measurements obtained by the accelerometer sensor. The front-end module may interact with the back-end to transmit the received measurements 9106 and obtain results of the evaluation. The front-end module may also be interacting with the user, to further output 9108 the results of the evaluation to the user on a graphical user interface of the smartphone. The back-end module may perform the evaluations and interact with the front-end to receive 9106 the plurality of accelerometer measurements from the front-end. The back-end may then transmit the evaluated output 9108 results for age, type 2 diabetes status and lifestyle-associated hazard ratio of the subject to the front-end.

FIG. 9.1 further shows the general pipeline of operations performed by the front-end and the back-end of the apparatus of the present example. Front-end is implemented in the form of a mobile application. The smartphone user may download and install the mobile application through one or more types of applicable App Stores (e.g., Apple App Store, Google Play Store). After installing the mobile application, the user may create a personal account and fill in the necessary personal information. The user may then proceed to login 9110 to the apparatus system. The user may also be prompted to fill in additional fields, such as “birthdate” and “gender”.

To complete the installation, the mobile application may prompt the user to grant the front-end permissions to access one or more of the system level services of the operating system (e.g., the CoreMotion 9102 and Location service 9103 services for the iPhone operating system). In one embodiment, the user may be using an iPhone, and this step may be required to receive measurements obtained from accelerometer sensor. While data on motion annotation and location are not directly used in the present example, access to the Location service 9103 and CoreMotion services 9102 may still be required for the data accumulation module 9111 to maintain access to the accelerometer sensor of the smartphone. In particular, the use of Location services may only be required to keep the mobile application processes from being killed or terminated by the operating system.

After the installation is complete and the necessary permissions are granted, the apparatus 9101 starts receiving and accumulating accelerometer measurements. Data accumulation module 9111 performs checks to ensure that the received acceleration measurements are valid. For example, it may check to see if the measurements are at least 2 minutes long and are recorded with a frequency of approximately 50 Hz. When a calendar day is over, module 9112 may transmit and upload the accumulated accelerometer data to the back-end 9100 using appropriate APIs, as defined by back-end. The received data are then preprocessed by the data quality filter 9114, processed by the Evaluation module 9116 and stored in a database 9118. All data can be associated with an anonymized user id and stored on the back-end with HIPAA compliance. In some embodiments, API of the cloud back-end is constructed with Django web-framework for the Python programming language using Amazon Web Services cloud infrastructure, and data is stored partially in a relational database, which is PostgreSQL, and partially in file storage, which is AWS Simple Storage Service. Variations of the implementation are possible, wherein the database 9118 can be a relational database, non-relational database, file storage and many other suitable storage systems, and combinations thereof. These exemplary variations and many other variations do not change the overall architecture of the disclosed apparatus.

FIG. 9.2 shows a more detailed illustration of the procedures according to an embodiment described herein. The figure illustrates a general pipeline of the procedures performed by the Receiving and accumulating data module 9111 for receiving measurements, performing data validity checks, and accumulating daily accelerometer measurements. Acceleration measurements are received by the front-end application using operating system API and recorded to a storage on the smartphone. Acceleration measurements are recorded in 2 to 5 minute chunks, in order to save battery power and ensure uniform sampling of acceleration throughout the day. The front-end applies several filters to ensure that received accelerometer measurements correspond to user's motion and exclude periods when the iPhone is not moving. First, the application does not start recording until it receives a signal from CoreMotion service 9102 (Step 9205), which indicates that smartphone motion has been detected. If the signal indicating that the smartphone's motion is active for longer than 3 seconds, the application may start receiving and recording acceleration along three spatial axes using operating system API (Step 9220). The recording is stopped when either the total measurement series duration exceeds 5 minutes or the signal from Core Motion service indicates that the smartphone is not in motion for longer than 5 seconds (Step 9225).

While receiving accelerometer measurements, the front-end may perform initial checks to ensure data integrity and correctness, and to further ensure that collected data can be used for evaluations, as described in present disclosure. In particular, after recording is stopped the collected chunk of measurement points may be checked for the following: whether 1) the length of a chunk is at least 2 minutes and 2) the sampling frequency is 50±2 Hz, according to the actual timestamps of collected data points. If both conditions are met, the chunk of data measurements may be stored in JSON format to be further uploaded to the back-end (Step 9235). In one embodiment, the front-end uploads collected data to the back-end, when Wi-Fi network is available. The presented algorithm aims at collecting at least 2 hours of accumulated accelerometer measurements per day, distributed uniformly throughout the day. Modifications to the algorithm can be made without limiting the scope of the example, including, for example, continuously collecting measurements during the day (in case battery performance is not an issue).

Preprocessing procedures are performed by the back-end, after accelerometer data for a single week is uploaded. The back-end software, implemented in the form of a scripting language (e.g., Python), performs pre-processing on the collected data to filter out data chunks which do not meet the minimal requirements of the quality filter module 9114. To inform the user about the estimated amount of accumulated data, the progress of collecting acceleration measurements may be reported in the form of a progress bar 9120 on the dashboard of the mobile application; the amount of progress may be incremented when the obtained data (e.g., plurality of measurements) meet the data quality filter 9114 requirements.

The data quality filter 9114 may check whether the collected plurality of measurements is valid for applying the method described in Example 4 for evaluating age and hazard ratio. For this purpose, the accelerometer measurements along the x-axis can be converted to activity counts according to the following algorithm: each series of measurements is assumed to be sampled at 50 Hz and is passed through a “boxcar” rectangular-shaped band pass filter with the lower and upper frequency bounds corresponding to 2.5 and 12.0 Hz, respectively. Activity count can be calculated in 1-minute intervals, by taking the sum of absolute values of filtered acceleration and multiplying the resulting sum by 0.02. The measurements may be analyzed on a daily basis and only those days for which total activity count is greater than 100 and the largest rest interval—e.g., interval with no activity counts is less than 10 hours—may be retained. If more than 7 days' worth of activity count data meet the requirements of the Quality filter, then the measurements of the last available 7 days can be used for feature extraction and evaluation of age and hazard ratio.

If the user filled in the date of birth field and is 45 years old or above, quality filter module 9114 may additionally check whether collected plurality of measurements is valid for evaluation of age and type 2 diabetes status according to Examples 1 and 2, respectively. The quality filter checks whether the total amount of collected measurements corresponds to 1) at least 2 hours per day and 2) a total of at least 7 days. Next, collected records are split into one-minute long intervals and absolute value of the acceleration vector and power spectrum density (PSD) is calculated for each interval using Welch method with window length of 1024 points. For each interval a numerical characteristic is calculated; the numerical characteristic may be equal to the natural logarithm of the sum of PSD values at 512 discrete frequencies. If the number of intervals for which the said characteristic is in the range between 2.0 and 2.5 is greater than 10%, the weekly update of collected plurality of measurements is considered valid. If at least 14 hours of data that meet the requirements of the Quality filter are accumulated, then the evaluation of age and type 2 diabetes status is performed.

Evaluated age 9121, type 2 diabetes status 9122, obesity 9123 and hazard rate or hazard ratio 9124, are stored at the back-end, and the front-end mobile application fetches the updated evaluations from the back-end using back-end APIs (either on user request or on a regular basis) and outputs fetched evaluations to the user.

In some embodiments, evaluated indications are fetched from the back-end along with additional information, for a more comprehensive visualization. For this purpose health score 9125 can be calculated based on hazard ratio, and outputted to the user (9505) as shown in FIG. 9.5. Evaluations can be fetched from the back-end and outputted for display on a separate screen on the mobile application. Additional information may also be outputted for a more comprehensive visualization, as described below.

FIG. 9.3 shows an example screenshot 9300 of an output for evaluated age. Evaluated age can be outputted as “Biological age” 9305, along with additional information to demonstrate how user's evaluated age compares to one's chronological age. The “optimal range” 9325 of evaluated age is shown, and a user within the optimal range implies that physical activity of the user corresponds to majority of the population having actual age in the said range. The lower 9310 and upper 9315 bounds of the “optimal range” correspond to the root mean square deviation for age evaluation (e.g., ±6 years in the present example). If the user filled in the date of birth field, his or her actual (i.e., chronological) age 9320 may also be shown for comparison with evaluated age. If the user's actual age is greater than “optimal range”, the user may be notified that physical activity performance is “excellent” and if the user's actual age is below the “optimal range” 9325, the performance may be depicted as “poor”.

On the same screen of mobile application, level of physical activity data collection may be displayed. Progress bar 9330 visualizes the amount of data collected, wherein 100% may correspond to 7 days of at least two hours per day of collected activity, when activity is uniformly collected throughout the day. Information button 9335 may provide user with explanation on how progress of data collection is measured and visualized on the progress bar.

FIG. 9.4 shows an example screenshot of an output for evaluated type 2 diabetes status. The sample screenshot illustrates the evaluated type 2 diabetes status 9405 via as an arrow 9410. The arrow may represent a continuous value in the range from 0 to 1, and may point to one of three labels 9410: “low”, “medium” and “high”. Additionally an estimation of Body Mass Index (BMI) is shown 9415 displaying the estimation in the form of one of the ordinal labels: “S”, “M”, “L”, “XL”, “XXL”. The “medium” label may denote a borderline state, “high” implies elevated risk of prediabetes, wherein high risk may imply a potential advice to pay more attention to the physical performance of the user or to attend a physician for appropriate testing to check evaluation results. Diabetic score is calculated according to probability of having positive type 2 diabetes status, as evaluated by the back-end according to methods described herein. If said probability is below 0.3, “low” risk is reported. “Medium” risk is reported if said probability is between 0.3 and 0.7, and “high” risk is reported if said probability is above 0.7.

FIG. 9.5 shows an example screenshot of an output for evaluated lifestyle-associated hazard ratio (“Health Score”) according to embodiments described herein. In some embodiments, evaluated hazard ratio (e.g., representing the probability of death before 60 years old) falls into the range between 0.0 and 10.0, wherein the value of 1.0 indicates population average probability. For the purpose of more comprehensive representation, the hazard ratio is converted into logarithmic scale of base 10, clipped to range between −1.0 and 1.0, and linearly rescaled to the range between 0.0 and 100%. The resulting value is reported as “health score” and may be visualized as, for example, the remaining battery level 9505 between 0 and 100%. Additionally, health score trend information for previous days can also be shown to the user in the form of trend-graphs or bar-graphs 9510. Trends may be shown for the week or month 9515.

FIG. 9.6 shows an example screenshot of an output for an alternate presentation of evaluated hazard ratio (“Lifestyle Hazard Ratio”). Evaluated hazard ratio may be outputted as “Lifestyle hazard ratio” 9605, along with relevant statistics. The evaluated hazard ratio of the user 9610 is plotted on a bar graph, where the horizontal axis (“relative risk”) 9615 may depict the user's hazard ratio relative to population average. The vertical axis (“reference participants”) 9620 may additionally depict the number of participants of reference dataset 9625 for a given hazard ratio. A threshold cutoff 9630 is depicted representing a significantly elevated hazard ratio of 1.5. The advantage of such representation is that it provides a way to estimate the average hazard ratio for a group of subjects. For example, checkbox selecting “smoking” subpopulation in the “lifestyle categories” menu 9635 shows distribution of hazard ratios of reference dataset subpopulation 9640 with self-reported smoking more than 5 cigarettes a day to additionally compare hazard ratio of the user against the said subpopulation. Other populational comparisons can be performed, using a group of checkboxes of the “lifestyle categories” menu 9635.

Comparison of the subject's physical activity characteristics with populational distribution opens possibilities for assessment of public health. The users can be grouped by gender, lifestyle factors, and these subpopulation groups can be further compared in terms of physical activity performance. This information, accumulated and stored in database 9118 can be useful for review by authorized third parties, including public healthcare and health insurance agencies.

Many modifications can be made to the present example. For example, other smartphone can be used, and correspondingly other mobile operating systems, such as Android or Microsoft or other operating system. In such modifications, software implemented system of the present example should use services, as provided by operating system, which are equivalent to Location and CoreMotion services of iPhone operating system. In some cases, different set of services can be used, provided said set allows to collect the same or similar amount of acceleration measurements with similar or same quality, as described in present example, and some services can be used solely for proper functioning of mobile application, which is used to collect accelerometer measurements.

Example of software-implemented apparatus, as described in the present example, can also contain additional web service, or API entry points, for providing access to evaluations of a user or a group of users to third party, such as physician or insurance company.

Parameters of data collections can be modified, for example, more than one week of activity may be collected in order to increase the signal-to-noise ratio and improve accuracy of evaluation. In many embodiments, wherein the invention is implemented in software, as described in present example, different age- and health-related indications are reported to user, and in many instances, user chooses the indications to be outputted. Reported indications can be presented in many different ways, as numbers, charts, bars and other graphical representations, and specific way of representation does not limit the scope of the example.

Back-end can be implemented in various ways, and person skilled in the art would appreciate the wide range of software technologies and infrastructures, which can be used for construction of back-end of the present example. Present example can, therefore, be implemented as a system comprising mobile application and cloud or web back-end on wide range of smartphones, using various infrastructure and programming technologies. Parameters of back-end and mobile application, which determine, how data is collected and stored, evaluation is performed, outputted to the user, and how evaluations are stored, are reconfigurable, and can vary, according to technical limitations, specific infrastructure, on which apparatus is implemented, and other implementation details.

### Example 7 (Fitbit Widget)

The example shows how the above described methods for evaluating age and hazard ratio based on step count measurements can be implemented in an apparatus. In the present example the subject is a user of a wearable device such as the Fitbit fitness wristband. The signal produced by the wristband is received in the form of time-ordered plurality of measurements of the user's step counts per minute, while the wearable wristband is supposed to be externally coupled to the user. Again, as in the previous example, the free-living physical activity in the present example implies that the user's behavior is uncontrolled and the apparatus is only passively measuring physical activity without requiring the user to perform any special actions. The apparatus updates the evaluated age on user's request to further output it to the user in visual form using web-interface.

The example further illustrates how additional metadata on the subject's gender can be used to improve the accuracy of evaluation of the subject's age, provided that information on gender is commonly readily known by the subject. The details of the apparatus and pipeline of operations are described below.

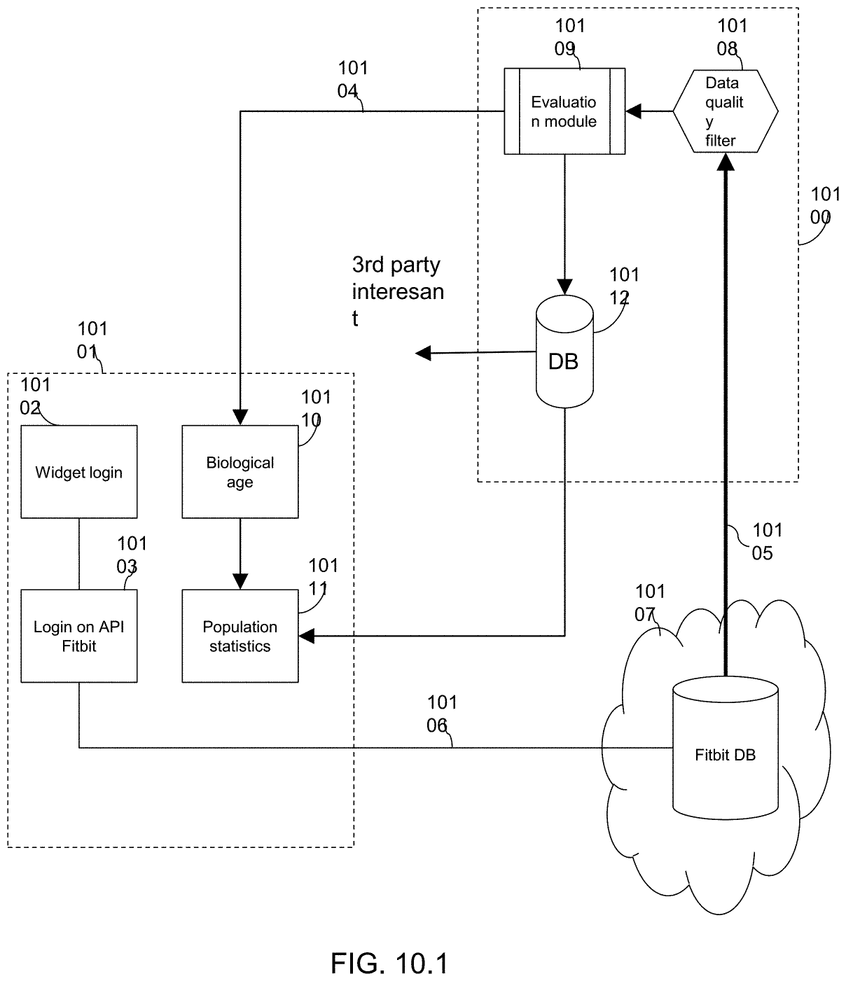

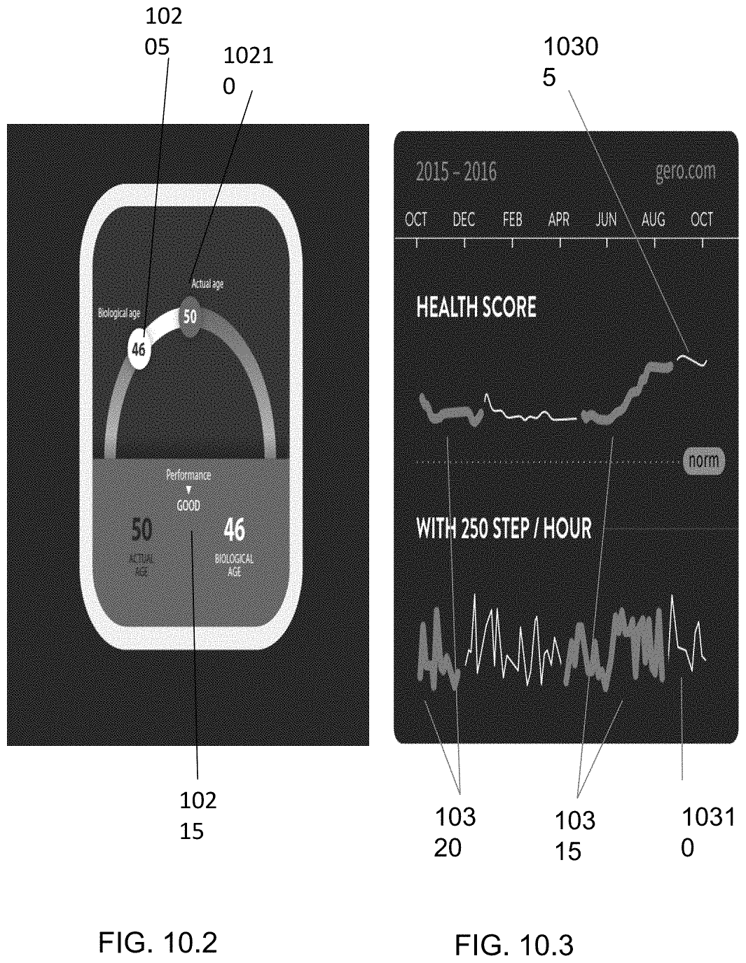

FIG. 10.1 shows the general architecture of apparatus which comprises a software-implemented system, wherein the system comprises a back-end module 10100 and a front-end module 10101 both running on server. The front-end module is interacting with the user to obtain login to the apparatus system 10102 and to access 10103 to user's step count measurements stored on Fitbit server and to output 10104 the evaluated age to the user in a graphical way using web-interface. The back-end module performs the evaluations and interacts with the front-end and Fitbit server to receive 10105 the plurality of accelerometer and transmit the evaluated age for outputting 10104 to the front-end.

FIG. 10.1 further shows the general pipeline of operations performed by the front-end and the back-end of the apparatus of the present example. To start using the invention, the user of Fitbit wristband should already have a valid Fitbit account and should create a personal account using the front-end web-service widget 10101 and fill in the required fields password and login 10102 and is then prompted to optionally fill in additional fields “birthdate” and “gender”. Next, using Fitbit API, the apparatus receives 10105 the measurements of step counts per minute according to the following procedure. The user is forwarded to Fitbit server and is prompted to grant one session access to step count measurements of the user's Fitbit account 10106 which are stored at Fitbit server 10107. Thereby, the plurality of measurements of the subject's free-living physical activity in this example is the plurality of measurements of step counts per minute, annotated by timestamps with 1 minute resolution.

After the user has authorized the access 10106, the data are downloaded from Fitbit server to the back-end using appropriate API, as defined by back-end for further preprocessing by Data quality filter module 10108 and evaluation of the age of the user by the Evaluation module 10109. Back-end API is implemented with Django web-framework, using Amazon Web Services, and in particular AWS Elastic Compute Cloud and AWS Simple Storage Service. Data is stored in relational database, which is PostgreSQL. Modifications to architecture of back-end are various and person skilled in the art will immediately appreciate other architectures, which are capable of performing the same tasks of data receiving from FitBit through appropriate API, as well as data storage, for example, in relational, nonrelational or file storage, and evaluation of age using method of present disclosure, and further outputting evaluated age 10110 to user through website of the front-end module.

After the step count measurements obtained from Fitbit sensor representing the subject's free-living physical activity are received and uploaded, the back-end software implemented in the form of a scripting programming language (e.g., Python) splits the measurements into slices of daily measurements and performs preprocessing to filter out daily data slices which do not meet the minimal requirements of the Quality filter module 10108. The quality filter checks whether each daily record of step count measurements has cumulative number of steps greater than 1000 and whether the longest continuous interval of rest is less than 10 hours. The interval of rest is defined as the continuous interval with steps less than cutoff value of 20 steps per minute. Finally, the quality filter checks whether the accumulated plurality of measurements corresponds to at least 7 days of step count measurements. If the received plurality of step count measurements meet the requirements of the Quality filter module 10108, the measurements are used for further preprocessing, feature extraction and evaluation of age of the user according to the procedures of Example 3 of the present disclosure.

Additional characteristics of the user's physical activity may also be evaluated in the present example for the purpose of visualization and a more comprehensive understanding of the evaluated age. The said characteristics represent aggregate characteristics of the user's physical activity and are calculated in the following way. First, averaged natural logarithm of power spectrum density (PSD) may be calculated based on plurality of step count measurements using Welch method with window size 512. The calculated average natural logarithm of the PSD is linearly projected onto two vectors. The two vectors may represent two principal component directions of the reference dataset. The linear projection yields two numerical values, one of which is interpreted as the total spectrum power and the second is interpreted as the power law coefficient of the dependence of spectrum density on the frequency. These two numerical characteristics are known to represent overall activity value and behavioral flexibility of the subject, relative to the population 10111, respectively. The evaluated age along with additional characteristics of the user's overall physical activity and behavioural flexibility are stored at database 10112, provided the user has given written informed consent, and are transmitted to the front-end web service widget and are outputted to the user in the visual and interactive way as described in more details below.

FIG. 10.2 shows an example screenshot of an output for evaluated age. Evaluated age is outputted as “Biological age” 10201. If the user filled in the date of birth field, his or her actual (chronological) age 10202 is shown for comparison with evaluated age. If the user's actual age is greater than the evaluated “biological age”, the user may be notified with qualitative estimate that physical activity performance is “excellent” and if the user's actual age is below the “optimal range”, the performance may be depicted as “poor” 10203.

The method for evaluating hazard ratio of the subject described in Example 4 can be used to find or gain insight into associations between trends in hazard ratio and changes in human lifestyle habits such as achieving a certain level of total number of steps per day, number of hours with more than 250 steps, or sleeping time, etc. The hazard ratio may be calculated for each seven consecutive days based on a plurality of measurements of freely moving physical activity obtained from the sensor coupled (e.g externally or otherwise coupled) to the subject. In this particular example, lifestyle is quantitatively measured as the number of hours (during a day) with more than 250 steps. This lifestyle activity goal may be calculated for each day based on the same plurality of measurements. After this calculation there may be two time series, one of hazard ratio and lifestyle activity, and one of hazard ratio corresponding to the last day of the seven days used for evaluation thereof. Each of calculated time series of hazard ratio may be subjected to low-pass filtering procedure. A low pass filtering procedure may drop out all frequencies higher than 1/604800 Hz from the time series. After low-pass filtering each time series x_{f}(t) may be divided into monotonic intervals (trends) and then the trends having length more than 10 days and at which variance of |x_{f}(t)−x_{f}(t)| is less than |x_{f}(tₛₜₐᵣₜ)−x_{f}(t_{end})|, where tₛₜₐᵣₜ and t_{end} are start and end dates of monotonic intervals, respectively, may be selected. Then the trends of hazard ratio and lifestyle activity time-series may be collected into pairs such that each pair includes one trend from hazard ratio time series and one from lifestyle activity time series and their overlap is more than 0.7 of length. After selecting trends, raw time series of negative value of hazard ratio 10305 and lifestyle activity 10310 may be presented in two plots and each pair of trends 10315 or 10320 may be highlighted with same color or pattern as shown in FIG. 10.3. The value of minus hazard ratio 10305 may be named as Health Score, and can be interpreted by the user as a wellness parameter. The highlighted trends of Health Score (e.g. wellness parameter) and lifestyle activity may show associations between their dynamics for the subject. Showing associations between changes in Health Score (e.g. wellness parameter) and lifestyle activity may be the basis of a system for producing recommendations and/or insights into lifestyle or living environment intervention to improve the subject's health. For example, FIG. 10.3. shows that Health Score 10305 has an ascending trend 10315, suggesting further increasing of the Health Score. Also, Health Score 10305 and lifestyle activity 10310 have similar trends—descending 10320 and ascending 10315, suggesting that for this particular subject increasing of life activity status 10310 leads to an improved health.

Possibility to compare characteristics of the subject's physical activity with populational distribution may be beneficial for assessment of public health. Users can be grouped by lifestyle factors and these subpopulation groups can be further compared in terms of physical activity performance. Such information, accumulated and stored in a database 10112, can be useful for review by authorized third parties, including public healthcare and health insurance agencies.

Many modifications can be made to the present example. For example, as would be appreciated by a person skilled in the art, many commercially available fitness-trackers can be used instead of Fitbit fitness-tracker and the choice of a specific fitness-tracker does not limit the scope of invention. Fitness trackers which may have API to access collected data can also be used in an implementation of the present example.

In case a fitness-tracker with different settings is used, for example, fitness-tracker reporting step counts per 5 or more minutes, back-end should use a different pipeline for age evaluation, which is similar to disclosed pipeline, but may have different settings, and, potentially, different model for age evaluation. For example, the back-end should use the model suitable specifically for evaluation of age based on the specific step counts data, as reported by tracker used for data collection. For example, back-end can use age model, which is constructed as regularized linear regression, as for FitBit tracker, but with different size of transition matrix. Possible models of present example are similar in architecture, and all of them implement method, as described in examples above, and extensions to more complex models are obvious to a person skilled in the art.

In some instances, additional mobile applications can be used, which may be used in place of a web-interface and implements the same functionality. In many instances, wellness evaluation can be performed on a regular basis, for example, each week, or each month, or more, or in between, as needed and requested by user. In such a case, evaluation is performed using updated step counts data, which includes step counts data, collected after previous evaluation.

Evaluated wellness indications can be presented in many ways, for example, textual, numerical or graphical and specific way of representation does not limit the scope of the example. Although presented with FitBit fitness tracker, present example provides a general architecture and flows of operation, making extensions of the present system for other fitness-trackers and cloud architectures rather trivial. Present example can, therein, be implemented in various ways, using various commercially available fitness-trackers and on various infrastructure.

### Example 8

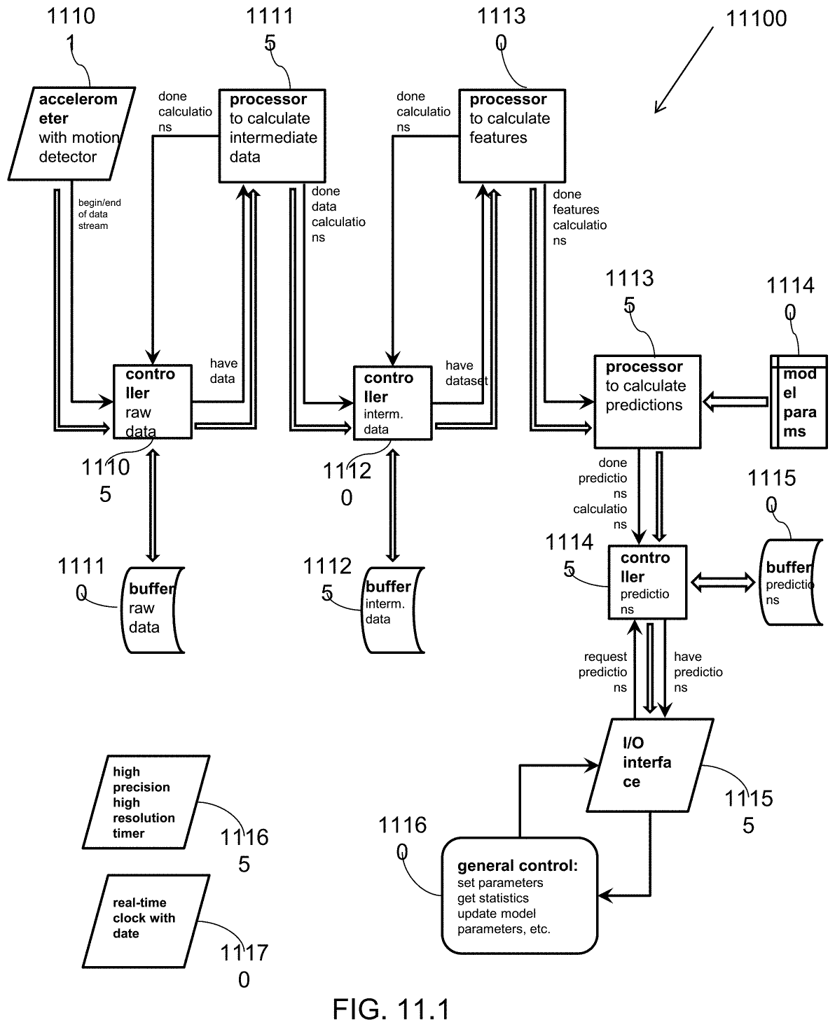

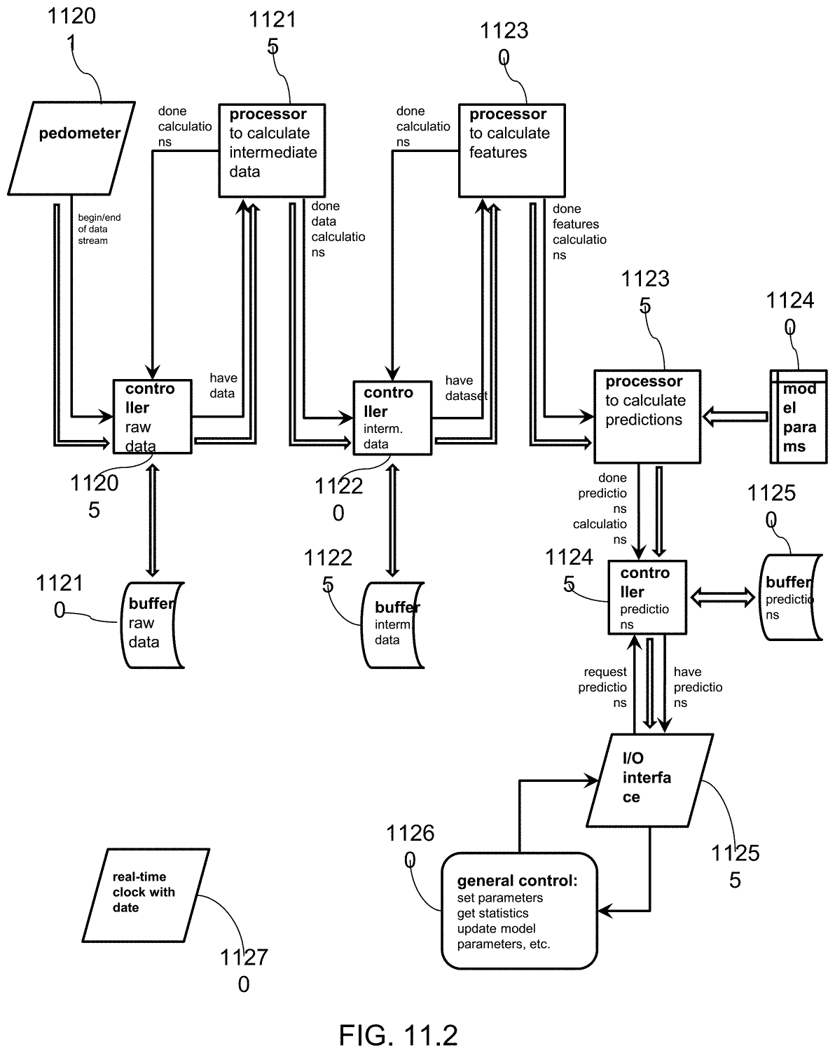

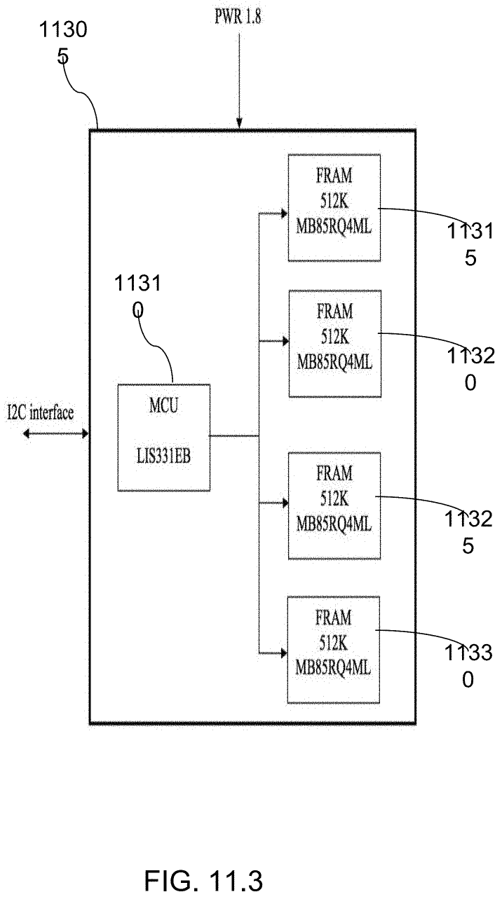

FIG. 11.1 shows an example schematic diagram of an electronic module implementing methods and systems described herein. In some embodiments, the sample electronic module 11100, when coupled to a subject during freely moving physical activity, receives a plurality of measurements of physical activity, extracts a plurality of features from received plurality of measurements and evaluates wellness indications, according to some embodiments described herein. The sample electronic module 11100 may be implemented into a form of a processing circuit. The module 11100 may be integrated into a wearable device.

In some instances, the accelerometer sensor 11101 continuously measures acceleration along at least one axis. In other instances, the accelerometer measures acceleration along two or three spatial axes. A signal is transmitted to the Controller 11105 when acceleration recording starts. Upon receiving the signal from accelerometer 11101, Controller 11105 may begin to save acceleration measurement data points into the buffer 11110. The data points saved into the buffer may be raw data output by the accelerometer. The accelerometer 11101 may send a signal to the Controller, indicating the end of recording. On receiving the end of recording signal, Controller 11105 may send a signal to the processor 11115. The signal may indicate that the acceleration measurements record is available, as defined by corresponding signals from the accelerometer. In some instances, begin and end signals from the accelerometer 11101 indicate the begin and end of series of sequential acceleration measurement records of predefined time duration.

Upon receiving signal from Controller 11105, processor 11115 starts to perform calculations on data received from Controller 11105. The calculations may comprise one or more of: 1) calculation of the magnitude of acceleration; 2) transformation of raw data (accelerometer output) to floating point format of predefined precision; 3) performing checks on the raw data, such as, for example, checking whether the raw data values are non-zero and valid according to predefined threshold levels; 4) downsampling the raw data; and 5) other operations and calculations required to transform the raw data for further calculations. In other embodiments, operations performed by the processor 11115 may be performed in various different combinations of sequential order, and one or more of the aforementioned operations may be omitted.

When processor 11115 completes the calculation, new intermediate data may be created from the raw data. The processor may send a signal to the Controller 11105 to indicate completion of calculation. In some instances, Controller 11105 may flush the buffer 11110 upon receiving such signal from processor 11115. When processor 11115 completes the intermediate data calculations, it may also send a signal to Controller 11120 to indicate that new intermediate data is available. Upon receiving the signal and the intermediate data from processor 11115, Controller 11120 may store the intermediate data in the buffer 11125. When one or more predefined requirements on amount of intermediate data is satisfied, Controller 11120 sends signal to processor 11130 to indicate that intermediate dataset is ready for further calculations.

In some embodiments, the controller 11120 may signal the processor 11130 when a predefined number of series of measurements are available in the buffer 11125. In other embodiments the pre-requisite for signaling may be based on the total number of measurement data points or predefined total time duration of the series of measurements. Other predefined conditions may be possible, according to specific evaluation type or implementation. The predefined condition may ensure that a plurality of features may be extracted from the intermediate data, collected in conformity with predefined condition. In some embodiments, one or more sets of predefined conditions may be used as necessary, for a specific evaluation type or implementation.

In some embodiments, raw data buffer 11110 is large enough to store enough data, as needed for extracting plurality of features, so that part of pipeline, working on intermediate data, such as processor 11115, controller 11120, intermediate buffer 11125 are not present in the module 11100, while processor 11130 performs all the necessary calculations, in relation to previously set. In other embodiments there may be several intermediate data buffers, with design either similar to or different from buffer 11125.

Upon receiving signal from controller 11120, processor 11130 starts extracting features from available intermediate data, wherein type of features extracted is determined by the specific evaluation type or implementation. In some instances, processor 11130 calculates the PSD of intermediate data and optionally applies logarithm to each component of power spectrum density. When processor 11130 finishes extraction of features, it sends a signal to processor 11135, wherein signal is indicating that feature extraction is finished.

Upon receiving a signal from processor 11130, processor 11135 evaluate wellness indication, wherein specific indication may be one or more of age, type 2 diabetes status, BMI, hazard rate, or hazard ratio or other as described in illustrative examples hereinabove. With the plurality of features, as extracted by processor 11130, processor 11135 performs evaluation using model parameters, which are stored in constant memory 11140. When processor 11135 finishes evaluation and has predictions available, it signals the controller 11145, which stores corresponding evaluated indication values in the buffer 11150. In certain embodiments the controller 11150 is configured to store the evaluated indications with time stamps of the end and start date and time of the period during which the acceleration measurements have been collected and processed.

All buffers, as described herein, may reside on either physically separate hardware components, or may be assembled on the same hardware component.

In many embodiments, model parameters may be hardwired in processor 11135 or in a separate hardware. In other embodiments, model parameters are stored on a non-rewritable memory, and in a yet other embodiments model parameters can be stored on rewritable non-volatile memory. In some embodiments, model parameters are stored in volatile memory, in case device is supposed to be never disconnected from power source during its functioning. Other implementations may be employed including those apparent to those of ordinary skill in the art, depending on implementation of other components, design requirements and other factors.

Controller 11145 also sends signals Input-Output interface 11155, wherein signal is indicating that new prediction is available. Upon receiving such signal with corresponding prediction, I/O interface outputs prediction and transmit and/or display it to subject by some means. In some embodiments, I/O interface displays wellness indication on a screen. I/O interface is also used to output wellness indication on demand, in which case a signal is sent to controller 11145, which outputs wellness predictions from buffer 11150, or pulls the prediction from previous components, as described herein. In some embodiments, predictions buffer 11150 is optional, thus, predictions are always pulled from upstream pipeline after receiving of a signal from I/O interface.

General control interface 11160 is used for control and setting up the device, including, but not limited to: setting device parameters, get statistics, update model parameters. In some embodiments, general control interface is optional, depending on specific implementation of other components of pipeline and wellness indications in consideration.

In order to maintain high precision time measurements, example device also comprises high precision high resolution timer 11165 and real-time clock/calendar 11170, coupled to all components, which need to account for time. In some embodiments, components 11165 and 11170 may be implemented in a single electronic component.

FIG. 11.2 shows an example schematic diagram of an electronic module which uses a pedometer, implementing methods and systems described herein. The pedometer 11201 produce a continuous stream of measurements, which are number of steps per given interval.

The raw data controller 11205 stores the pedometer data in circular buffer 11210. There are several time points programmed into the controller 11205, which split the day (24 hours) into N intervals. Once the said time point comes, as indicated by real-time clock 11270, the controller 11205 signals to the processor 11215 that the data are available and provides processor with the location of the pedometer data, corresponding to the interval preceding the time point which triggers the event.

The intermediate data are K×K integer-valued transitions-counting matrices C, stored in the intermediate values buffer 11225. Here K is the number of states as explained below. There are M·N such matrices: one matrix per per day per each of the N intervals of the day. Here M is the number of days used by the model to determine the wellness indicator. The transition-counting matrices are organized into a circular buffer.

The intermediate data are calculated by processor 11215. The transitions-counting matrix C is selected, based on date and time provided by the real-time clock 11270 and the matrix is set to zero. For each pedometer value taken from buffer 11210 the state number j is calculated according to the following rule: state 1 contains pedometer values p≤V₁, states 1<i<K contain values Vᵢ₋₁≤p<V₁, and state K contains values p≥V_{K-1}, where are K−1 values programmed into processor 11215. Given the just obtained state number j and the previous state number i the value Cⱼᵢ of transition-counting matrix is incremented by 1. The pedometer values are processed consecutively. Note that processor 11215 uses only simple integer arithmetic.

The feature used in this example is a set of N K×K real-valued transition matrices T. Once a day in a predetermined time based on the real-time clock 11270 readings, the controller 11220 triggers the calculation of the feature by processor 11230. The mentioned predetermined time is effectively considered to be the beginning of a day.

For each transition-counting matrix the sum S of all Cⱼᵢ over all j and i≥q is calculated. If for any of the matrices S<A, the data-quality warning flag F is set. Here q is the smallest state number still considered active and A is the minimum number of times the active state must be detected for the time interval be considered non-empty enough for wellness indicator estimation. The value of A is interval dependent: there are N values of A programmed into the processor 11230, one for each of the intervals day is divided into.

Each transition matrix with size. K×K (where K is the number of given bins) is calculated as follows. First, time series x is converted to time series y=[y₁, y₂, . . . ], where yₖ is the number of bin which contains xₖ. Then each cell is calculated as a number of n such that yₙ=j and yₙ₋₁=i. Finally each cell in divided by sum Wᵢⱼ over j and then sum Wᵢⱼ over j is subtracted from each diagonal cell Wᵢᵢ. Together, K×K values of N transition matrices forms the feature vector.

Once the feature vector is calculated, the wellness indicator estimation is calculated by processor 11235 using parameters stored in nonvolatile memory 11240. In implementation described in this example uses a simple linear model: the feature vector is multiplied element wise with the vector of coefficients, summed over and an interception coefficient is added. Linear model require N×K×K+1 coefficients per model. Several different wellness indicator can be estimated consecutively provided that they all use linear model described here and exactly the same feature vector.

The calculated wellness indicator estimations accompanied by the date from the real-time clock 11270 and the data-quality warning flag F are stored in the buffer 11250 by the controller 11245.

The control module 11260 allows important parameters of the apparatus to be setup. These parameters are: the frequency of pedometer output (must correspond to actual pedometer settings), the size of the raw data buffer (must be large enough to store raw pedometer data generated during a day), the the number of days M to be used by the models, the number N of the intervals within the day together with N time points to separate them, the number of states K together with K−1 values to separate them, the active state threshold q and N activity thresholds A, number of wellness indicators for which the estimations are to be provided together with N×K×K+1 coefficients for each of them and the time point when to calculate the feature vector and wellness indicator estimations. The control module also allows the real-time clock to be adjusted. The access to the intermediate buffers and reading of various parameters are not provided.

The I/O interface 11255 provides access to the buffer 11250 and interface to the control module 11260.

FIG. 11.3 shows an example circuit implementation of the apparatus and system described herein. The example described here is implemented around the STMicroelectronics LIS331EB signal processor 11310 featuring Cortex-M0 core with 64 KB Flash and 128 KB SRAM. In addition to the said processor, the apparatus also comprise the housing, the pedometer sensor chip, the rechargeable battery and an USB controller chip with an USB micro-B type receptacle. The built-in accelerometer of the LIS331EB is not used in this implementation.

The software used by the implementation is stored in the LIS331EB Flash. The LIS331EB internal RAM is used for all buffers and for storing intermediate results during calculations. The raw data are stored in the buffer 11210 using unsigned 16-bit integers, the transitions-counting matrices stored using 32-bit unsigned integers, the transition matrices and model parameters are stored in binary 32-bit floating point format, all real-valued calculations are performed using the same format. All processor are physically the same Cortex-M0 core of the LIS331EB. The controllers are implemented using the resources of the same core together with pointers stored in the chip memory using the timer-driven interrupts. In order to avoid data races the time to calculate the features shall not overlap with the period when the processor 11215 is working. This is achieved by offsetting the feature and estimation calculation from the nearest day-splitting point by 10 minutes.

Reference is made to the following claims which recite combinations that are part of the present disclosure, including combinations recited by multiple dependent claims dependent upon multiple dependent claims, which combinations will be understood by a person skilled in the art and are part of the present disclosure.

While preferred examples of the present invention have been shown and described herein, it will be obvious to those skilled in the art that such examples are provided by way of example only. It is not intended that the invention be limited by the specific examples provided within the specification. While the invention has been described with reference to the aforementioned specification, the descriptions and illustrations of the examples herein are not meant to be construed in a limiting sense.

Numerous variations, changes, and substitutions will now occur to those skilled in the art without departing from the invention. Furthermore, it shall be understood that all aspects are not limited to the specific depictions, configurations or relative proportions set forth herein which depend upon a variety of conditions and variables. It should be understood that various alternatives to the examples described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

The present invention also relates to the following embodiments (1)-(142):

1. A method to evaluate a wellness parameter of a subject in response to freely moving physical activity of the subject, the method comprising:

receiving a plurality of measurements of freely moving physical activity of the subject obtained by a sensor coupled to the subject;
extracting a feature from the plurality of measurements; and
evaluating the wellness parameter of the subject with a model in response to the feature, wherein the wellness parameter is selected from the group consisting of an age, a hazard rate, a hazard ratio, a type 2 diabetes status and a body mass index.

2. A method to evaluate a wellness parameter or a derived parameter of a subject in response to freely moving physical activity of the subject, the method comprising: evaluating the wellness parameter or the derived parameter with a model in response to the plurality of features, extracted from the plurality of measurements of freely moving physical activity of the subject received by a sensor externally coupled to the subject, wherein the wellness parameter is selected from the group consisting of an age, a hazard rate, a hazard ratio, a type 2 diabetes status and a body mass index, and wherein the derived parameter is evaluated in response to the evaluated wellness parameter and optionally wherein the derived parameter is evaluated in response to a plurality of evaluated wellness parameters.

3. A method to output or use a wellness parameter or a derived parameter of a subject, the method comprising outputting or using the wellness parameter or the derived parameter evaluated with a model in response to a plurality of features, extracted from a plurality of measurements of freely moving physical activity of the subject obtained by a sensor externally coupled to the subject, wherein the wellness parameter is selected from the group consisting of an age, a hazard rate, a hazard ratio, a type 2 diabetes status and a body mass index, and wherein the derived parameter is evaluated in response to the evaluated wellness parameter and optionally wherein the derived parameter is evaluated in response to a plurality of evaluated wellness parameters.

4. A tangible medium, configured with instructions that when executed cause a processor to perform the method of any one of embodiments [0545], 1 or 6.

5. An apparatus to evaluate a wellness parameter of a subject in response to freely moving physical activity of the subject, the apparatus comprising the processor comprising the tangible medium of embodiment 29.

6. The apparatus of embodiment 5, further comprising an activity sensor.

7. The apparatus of embodiment 5 or 6, further comprising:

a computer chip comprising the processor, the computer chip coupled to the activity sensor to receive the plurality of measurements from the activity sensor, the processor configured with instructions to extract a plurality of features from the plurality of measurements and output the wellness parameter of the subject, wherein the computer chip is configured to be carried by the subject and optionally comprises a length, a width and a height of no more than 5 mm, 5 mm and 3 mm, respectively.

8. A tangible medium to evaluate a wellness parameter of a subject in response to freely moving physical activity of the subject, the tangible medium configured with instructions that when executed cause a processor to,

receive a plurality of measurements of freely moving physical activity of the subject from a sensor coupled to the subject;
output the wellness parameter in response to the plurality of measurements with an output device, the output device selected from the group consisting of a display, an audio output, a haptic output and a brain computer interface, wherein the wellness parameter of the subject is selected from the group consisting of an age, a hazard rate, a hazard ratio, a body mass index and a diabetes type 2 status.

9. A method to evaluate a wellness parameter of a subject in response to freely moving physical activity of the subject, the method comprising:

receiving a plurality of measurements of freely moving physical activity of the subject obtained by a sensor coupled to the subject; and
outputting the wellness parameter, wherein the wellness parameter is selected from the group consisting of an age, a hazard rate, a hazard ratio, a type 2 diabetes status and a body mass index and optionally wherein the wellness parameter has been evaluated in response to a feature extracted from the plurality of measurements.

10. The apparatus, tangible medium, computer chip or method, as in any one of the preceding embodiments, further comprising:

evaluating a derived parameter with the model in response to a wellness parameters, the wellness parameter selected from the group consisting of an age, a hazard rate, a hazard ratio, a type 2 diabetes status and a body mass index and optionally wherein the derived parameter is evaluated in response to a plurality of wellness parameters selected from the group consisting of the age, the hazard rate, the hazard ratio, the type 2 diabetes status and the body mass index.

11. The apparatus, tangible medium, computer chip or method as in any one of the preceding embodiments, further comprising outputting the derived parameter and optionally outputting the wellness parameter used to evaluate the derived parameter and optionally outputting the plurality of wellness parameters used to evaluated the derived parameter.

12. The apparatus, tangible medium, computer chip or method, as in any one of the preceding embodiments wherein the feature is derived from frequencies of activity sensor data of no more than about 1 Hz and optionally wherein the feature is derived from frequencies above about 1 Hz.

13. The apparatus, tangible medium, computer chip or method, as in embodiment 12 wherein the feature is derived from activity sensor data having frequencies selected from the group consisting of no more than about 0.1 Hz, no more than about 0.01 Hz, no more than 0.001 Hz, no more than about 0.0001 Hz, no more than about 0.0002, no more than about 0.000011 Hz and no more than about 0.00001 Hz.

14. The apparatus, tangible medium, computer chip or method, as in embodiment 12 wherein the feature is derived from activity sensor data comprising an average power spectral density selected from the group consisting of no more than about 0.1 Hz, no more than about 0.01 Hz, no more than 0.001 Hz, no more than about 0.0001 Hz, no more than about 0.0002, no more than about 0.000011 Hz, and no more than about 0.00001 Hz.

15. The apparatus, tangible medium, computer chip or method, as in embodiment 12 wherein the feature comprises a plurality of features derived from frequencies from activity sensor data of no more than about 100 Hz.

16. The apparatus, tangible medium, computer chip or method, as in embodiment 12 wherein the feature comprises a plurality of features derived from frequencies from activity sensor data of no more than about 1 Hz.

17. The apparatus, tangible medium, computer chip or method, as in embodiment 16 wherein the plurality of features is derived from transitions among activity levels having frequencies selected from the group consisting of no more than about 0.1 Hz, no more than about 0.01 Hz, no more than 0.001 Hz, no more than about 0.0001 Hz, no more than about 0.0002, no more than about 0.000011 Hz, and no more than about 0.00001 Hz.

18. The apparatus, tangible medium, computer chip or method, as in embodiment 12 wherein the feature is derived from activity sensor data having frequencies within a range selected from the group consisting of any two of the following values: 0.1 Hz, 0.01 Hz, 0.001 Hz, 0.0001 Hz, 0.0002 Hz, 0.000011 Hz, 0.00001 and 0.000001 Hz.

19. The apparatus, tangible medium, computer chip or method of any one of the preceding embodiments, wherein the plurality of measurements comprises a series of data, wherein data of the series of data is separated by a time within a range selected from the group consisting of one millisecond to one second, one millisecond to one minute, one millisecond to one hour, one millisecond to one day, five seconds to one minute, five seconds to one hour five seconds to one day, one minute to one hour, and one minute to one day.

20. The apparatus, tangible medium, computer chip or method, of embodiment 19 wherein the plurality of measurements comprises of a low-resolution series of measurements.

21. The apparatus, tangible medium, computer chip or method, as in embodiment 19, wherein the plurality of measurements comprises a high-resolution series of measurements.

22. The apparatus, tangible medium, computer chip or method, as in any one of the preceding embodiments:

wherein the plurality of measurements comprises a series of measurements selected from the group consisting of a low-resolution series of measurements and a high-resolution series of measurements;
optionally wherein the low-resolution series of measurements represents a low resolution time evolution profile of physical activity levels of the subject with a time resolution longer than 1 s and optionally with the time resolution within a range from 5 seconds to 1 hour;
optionally wherein the high-resolution series of measurements represents a fast time evolution profile of a physical quantity related to motion of the subject with the time resolution shorter than 1 s and optionally with a time resolution shorter than 250 ms.

23. The apparatus, tangible medium, computer chip or method, as in any one of the preceding embodiments, wherein a shortest time resolution of the plurality of measurements is 0.01 s and optionally wherein the shortest time resolution of the plurality of measurements is 0.05 s.

24. The apparatus, tangible medium, computer chip, or method as in any one of the preceding embodiments, wherein each high-resolution series of measurements has time resolution selected from the group consisting of shorter than 100 ms, shorter than 50 ms and shorter than 20 ms.

25. The apparatus, tangible medium, computer chip, or method as in any one of the preceding embodiments, wherein the plurality of measurements comprises a plurality of measurements that meet requirements of preprocessing filtering, and optionally wherein the requirements correspond to the total accumulated amount of a low-resolution series of measurements, and optionally wherein the requirements correspond to a total accumulated daily amount of measurements sampled on one day from the subject, the total accumulated daily amount within a range from 8 to 16 hours after the subject woke up.

26. The apparatus, tangible medium, computer chip or method, as in any one of the preceding embodiments, wherein a total accumulated amount of a set of low-resolution series of measurements corresponds to at least about 12 hours of activity, at least about 24 hours, at least about 48 hours, and at least about 72 hours; and optionally wherein an interval between a first measurement and a last measurement of the total accumulated amount is selected from the group consisting of at least about 1 day, at least about 3 days and at least about 1 week and optionally wherein the total accumulated amount corresponds to freely moving physical activity over a plurality of days.

27. The apparatus, tangible medium, computer chip, or method as in any one of the preceding embodiments, wherein a set of low-resolution series of measurements comprises measurements sampled within a range from 8 to 16 hours after the subject woke up and a total accumulated amount of low-resolution series of measurements sampled within the range from 8 to 16 hours after the subject woke up corresponds to at least 4 hours of freely moving physical activity, and optionally wherein the total accumulated amount within the range corresponds to amounts measured over a plurality of days and optionally wherein the total accumulated amount is selected from the group consisting of at least 8 hours and at least 24 hours.

28. The apparatus, tangible medium, computer chip, or method as in any one of the preceding embodiments, wherein a set of high-resolution series measurements corresponds to at least about 3 hours, at least about 6 hours, at least about 12 hours, at least about 24 hours of freely moving physical activity of the subject; and optionally wherein an interval between a first measurement and a last measurement of the set of high resolution series measurements is selected from the group consisting of at least about 1 day, at least about 3 days, and at least about 1 week and optionally wherein the total accumulated amount corresponds to freely moving physical activity over a plurality of days.

29. The apparatus, tangible medium, computer chip, or method as in any one of the preceding embodiments, wherein a set of high-resolution series of measurements comprises measurements sampled between 8 and 16 hours after the subject woke up and total accumulated amount of high-resolution series of measurements sampled between 8 and 16 hours after the subject woke up corresponds to at least 3 hours of freely moving physical activity, and optionally corresponds to at least 6 hours of freely moving physical activity, and optionally corresponds to at least 12 hours of freely moving physical activity and optionally wherein the total accumulated amount within the range corresponds to amounts measured over a plurality of days.

30. The apparatus, tangible medium, computer chip, or method as in any one of the preceding embodiments, further comprising a preprocessing procedure to filter out series of measurements which span too short time range, or have inappropriate time resolution, or correspond to activity level period of inappropriate length, or otherwise are not appropriate for the evaluating of the wellness parameter.

31. The apparatus, tangible medium, computer chip, or method as in any one of the preceding embodiments, further comprising:

preprocessing the plurality of measurements with a transformation to generate output comprising a low-resolution series of measurements, transformation selected from the group consisting of calculating average, counting patterns of motion, switching to frequency domain and alike; and
optionally calculating desired activity level values in response to the low-resolution series of measurements;
optionally wherein the plurality of measurements comprises a plurality of series of measurements and each series of measurements undergoing the step of preprocessing spans a continuous time interval selected from the group consisting of at least 5 seconds long, at least 20 seconds long, at least 1 minute long, and optionally wherein said each series of measurements undergoing the preprocessing operation comprises a high-resolution series of measurements.

32. The apparatus, tangible medium, computer chip or method, as in any one of the preceding embodiments, wherein the plurality of measurements comprises a low-resolution series of measurements, the series of measurements comprising an interval between successive measurements of the series, wherein a physical activity level of the subject corresponds to a level of overall physical activity of the subject over a period of time, and wherein the period of time corresponding to the level of overall physical activity is not less than one tenth of the interval between successive measurements, not less than 5 s, and is not longer than ten times the length of the interval between measurements and is not longer than 1 hour.

33. The apparatus, tangible medium, computer chip or method, as in any one of the preceding embodiments, wherein the plurality of measurements comprises a low-resolution series of measurements, the series of measurements comprising an interval between successive measurements of the series, wherein a physical activity level of the subject corresponds to a level of overall physical activity of the subject over a period of time, and wherein the period of time corresponding to the level of overall physical activity is approximately equal to the interval between successive measurements.

34. The apparatus, tangible medium, computer chip or method, as in any one of the preceding embodiments, wherein a physical activity level of the subject during a period of time is selected from the group consisting of an integral characteristic of motion of the subject during said period, a number of specific patterns of motion during said period and a measured physiological quantity of the subject related to an amount of physical activity of the subject during said period;

wherein the integral characteristic of the subject motion is selected from the group consisting of an average, an area under curve, a total variation, a standard deviation and another similar characteristic of a physical motion signal, and wherein the integral characteristic is determined in a domain selected from the group consisting of a time domain and a frequency domain;
wherein the number of specific patterns of motion is selected from the group consisting of a number of steps, a number of steps of a specific type and a set of numbers of steps, wherein each number comprises a number of steps of a specific type or alike; and
optionally wherein the steps are classified according to a classification selected from the group consisting of upstairs steps, downstairs steps, walking steps, running steps and alike;
wherein the measured physiological quantity changes in response to the freely moving physical activity of the subject, wherein the physiological quantity is selected from the group consisting of a number of heartbeats, a peripheral oxygen saturation and another physiological parameter measured during an activity level interval, and.

35. The apparatus, tangible medium, computer chip or method, wherein the wellness parameter is output to a user within about an hour of receiving a last measurement received of the plurality of measurements from the sensor and optionally within about one minute of receiving the last measurement.

36. The apparatus, tangible medium, computer chip or method as in any one of the preceding embodiments, wherein the feature comprises a plurality of features.

37. The apparatus, tangible medium, computer chip or method as in any one of the preceding embodiments, wherein the feature comprises a single feature.

38. The apparatus, tangible medium, computer chip or method of any one of the preceding embodiments, wherein the feature, or the plurality of features is selected from the group consisting of time domain features, frequency domain features, and transition rates between different activity states.

39. The apparatus, tangible medium, computer chip or method of any one of the preceding embodiments, wherein the feature or the plurality of features comprises probability distribution properties or occupancy states of the plurality of measurements in a domain selected from the group consisting of a time domain and a frequency domain.

40. The apparatus, tangible medium, computer chip or method of any one of the preceding embodiments, wherein the feature, or the plurality of features comprise correlation properties of the plurality of measurements in a domain selected from the group consisting of a time domain and a frequency domain.

41. The apparatus, tangible medium, computer chip or method of any one of the preceding embodiments, wherein the feature or the plurality of features comprise correlation properties of the plurality of measurements, and wherein the correlation properties comprise autocorrelation in time domain.

42. The apparatus, tangible medium, computer chip or method of any one of the preceding embodiments, wherein feature or the plurality of features is selected from the group consisting of autocorrelation, power spectral density and transition rates between different activity states and probability distribution properties.

43. The apparatus, tangible medium, computer chip or method of any one of the preceding embodiments, wherein the transition rates between different activity states comprise a set of transition rates between activity states, and optionally wherein transition rates between different activity states comprise a matrix of transition rates between said activity states and optionally wherein said transition rates between different activity states comprise a full set of transition rates between all activity states and optionally wherein said transition rates between different activity states comprise the matrix of transition rates between all activity states.

44. The apparatus, tangible medium, computer chip or method, as in any one of the preceding embodiments wherein the output parameter is evaluated with a combined set of features comprising data from a transition matrix and a power spectral density from the plurality of measurements.

45. The apparatus, tangible medium, computer chip or method of any one of the preceding embodiments, wherein the tangible medium is configured with instructions selected from the group consisting of preprocessing the plurality of measurements prior to extracting the feature or the plurality of features and post-processing the feature or the plurality of features and optionally wherein said preprocessing or post-processing is performed prior to evaluating the wellness parameter.

46. The apparatus, tangible medium, computer chip or method of embodiment 45, wherein preprocessing comprises a procedure to filter the received plurality of measurements according to quality requirements for the wellness parameter being evaluated.

47. The apparatus, tangible medium, computer chip or method of any one of the preceding embodiments, further comprising preprocessing the plurality of measurements by performing a preprocessing operation.

48. The apparatus, tangible medium, computer chip or method of any one of the preceding embodiments, wherein preprocessing the plurality of measurements comprises determining whether the received measurements meet a quality requirement; and filtering out the measurements that do not meet the quality requirement.

49. The apparatus, tangible medium, computer chip or method of any one of the preceding embodiments, wherein the preprocessing operation is selected from the group consisting of down-sampling a series of measurements to lower resolution, calculating a length of vector physical quantity, splitting a series of measurements into slices of fixed or variable duration, filtering out slices of measurements with near-zero activity and logarithm scaling.

50. The apparatus, tangible medium, computer chip or method of any one of the preceding embodiments, wherein the preprocessing operation comprises a slicing operation that converts the plurality of measurements into a set of slices of predefined length along a time axis.

51. The apparatus, tangible medium, computer chip or method of any one of the preceding embodiments, wherein the sensor is selected from the group consisting of an accelerometer and a gyroscope and preprocessing the plurality of measurements comprises converting measurements of time evolution of acceleration or rotational quantity along individual axes into measurements of a time evolution selected from the group consisting of an absolute value of acceleration, an angular velocity, an angular acceleration, a rotational speed, and a rotational acceleration.

52. The apparatus, tangible medium, computer chip or method of any one of the preceding embodiments, wherein extracting a set of features from the plurality of measurements further comprises:

quantifying the plurality of measurements into bins of different activity states comprising binned measurements distributed among a plurality of bins;
analyzing the binned measurements in response to a statistical distribution among data points of signal levels of the binned measurements in each bin of the plurality of bins; and
calculating a transition rate between the signal levels of the measurements in each bin to yield an activity transition matrix, wherein said transition rate comprises a feature of the plurality of measurements.

53. The apparatus, tangible medium, computer chip or method of any one of the preceding embodiments, wherein the postprocessing procedure is selected from the group consisting of imputation of missing or near-zero values, logarithm scaling, and dimensionality reduction.

54. The apparatus, tangible medium, computer chip or method of any one of the preceding embodiments, wherein dimensionality reduction further comprises linear detrending or principal component analysis decomposition.

55. The apparatus, tangible medium, computer chip or method as in any one of the preceding embodiments, wherein the feature comprises a plurality of features and a single feature is extracted from the plurality of features and wherein the wellness parameter is evaluated in response to the single feature.

56. The apparatus, tangible medium, computer chip or method of any one of the preceding embodiments, wherein the single feature is extracted by dimensionality reduction of the plurality of features.

57. The apparatus, tangible medium, computer chip or method of any one of the preceding embodiments, wherein the feature or the plurality of features comprises a feature vector and the dimensionality reduction comprises a linear projection of the feature vector onto one or more vectors.

58. The apparatus, tangible medium, computer chip or method as in any one of the preceding embodiments, wherein the wellness parameter comprises a plurality of wellness parameters of the subject, the plurality of wellness parameters selected from the group consisting of the age, the hazard rate, the hazard ratio, the type 2 diabetes status and the body mass index and wherein the plurality of wellness parameters comprises a first wellness parameter and a second wellness parameter and wherein the second wellness parameter is evaluated in response to a combination of the first evaluated wellness parameter and the feature or the plurality of features

59. The apparatus, tangible medium, computer chip or method of any one of the preceding embodiments, wherein the plurality of measurements comprises a low-resolution series of measurements comprising a number specific patterns of motion during a period of time, and wherein an accuracy of the evaluated age corresponds to a Pearson correlation of about 0.55 or higher with actual age for a group of subjects with a uniform distribution of actual age in range from 20 to 70 years old and optionally wherein the Pearson correlation is within a range from about 0.55 to about 0.75, and optionally wherein the number of specific patterns of motion comprises a number of steps of the subject.

60. The apparatus, tangible medium, computer chip or method of any one of the preceding embodiments, wherein an accuracy of the evaluated age corresponds to a Pearson correlation of about 0.65 or higher with actual age for a group of subjects with a distribution of actual age in range from 20 to 70 years old and optionally wherein the distribution comprises a uniform distribution and optionally wherein the Pearson correlation is within a range from about 0.65 to about 0.85.

61. The apparatus, tangible medium, computer chip or method of any one of the preceding embodiments, wherein the accuracy of the evaluated age corresponds to Pearson correlation of about 0.7 or higher with actual age for a group of subjects with a uniform distribution of actual age in range from 40 to 70 years old and optionally wherein the Pearson correlation is within a range from about 0.7 to about 0.9 and optionally wherein the subject is a member of the group of subjects and optionally wherein the subject is not a member of the group of subjects.

62. The apparatus, tangible medium, computer chip or method, as in any one of the preceding embodiments wherein the evaluated age of the subject comprises a biological age.

63. The apparatus, tangible medium, computer chip or method of any one of the preceding embodiments, wherein the evaluated age of the subject is classified among a plurality of classes, the plurality of classes selected from the group consisting of young, adult, old and alike.

64. The apparatus, tangible medium or method, as in any one of the preceding embodiments wherein the age is evaluated without inputting an actual age of the subject.

65. The apparatus, tangible medium, computer chip or method of anyone of the preceding embodiments, wherein the wellness parameter comprises the diabetes type 2 status and wherein an accuracy of the evaluated diabetes type 2 status corresponds to a sensitivity and a selectivity selected from the group consisting of a sensitivity of at least 0.6 and at a selectivity of least 0.8, a sensitivity within a range from about 0.6 to about 0.9 and a selectivity within a range from about 0.8 to about 0.95, a sensitivity of at least 0.75 and a selectivity of at least 0.75, and a sensitivity within a range from about 0.75 to about 0.95 and a selectivity within a range from about 0.75 to about 0.95 and optionally wherein the accuracy is determined for a group of subjects and optionally wherein the subject is a member of the group of subjects and optionally wherein the subject is not a member of the group of subjects.

66. The apparatus, tangible medium, computer chip or method as in any one of the preceding embodiments, wherein the feature is associated with an age of the subject, and wherein the diabetes type 2 status of the subject is evaluated in response to the evaluated age of the subject combined with a body mass index of the subject and wherein the body mass index of the subject comprises the evaluated body mass index wellness parameter or a body mass index input from another source.

67. The apparatus, tangible medium, computer chip or method of any one of the preceding embodiments, wherein the evaluated diabetes type 2 status of the subject is classified among a plurality of classes, the plurality of classes selected from the group consisting of normal, borderline, diabetic and alike.

68. The apparatus, tangible medium, computer chip or method as in any of the preceding embodiments, further comprising evaluating hazard rate of a subject with the model in response to the evaluated hazard ratio of the subject combined with a reference hazard rate and optionally wherein the reference hazard rate comprises an average hazard rate of a reference population.

69. The apparatus, tangible medium, computer chip or method as in any of the preceding embodiments, further comprising evaluating hazard ratio of a subject with the model in response to the evaluated hazard rate of the subject combined with a reference hazard rate and optionally wherein the reference hazard rate comprises an average hazard rate of a reference population.

70. The apparatus, tangible medium, computer chip or method, as in any one of the preceding embodiments wherein an accuracy of the evaluated hazard rate or hazard ratio is greater than an ROC AUC of about 0.6 and optionally wherein the ROC AUC is within a range from about 0.6 to about 0.9 and optionally wherein the accuracy is determined for a group of subjects for which the ROC AUC is determined and optionally wherein the subject is a member of the group of subjects and optionally wherein the subject is not a member of the group of subjects.

71. The apparatus, tangible medium, computer chip or method, as in any one of the preceding embodiments wherein an accuracy of the evaluated hazard rate or hazard ratio is greater than a concordance index of about 0.6 and optionally wherein the concordance index is within a range from about 0.6 to about 0.9 and optionally wherein accuracy is determined for a group of subjects for which the concordance index is determined.

72. The apparatus, tangible medium, computer chip or method as in any one of the preceding embodiments, wherein the hazard ratio comprises a ratio of hazard rates between the subject and a reference hazard rate and optionally wherein the reference hazard rate comprises an average hazard rate of a reference population.

73. The apparatus, tangible medium, computer chip or method as in any one of the preceding embodiments, wherein the evaluated hazard rate or hazard ratio comprises a hazard rate or a hazard ratio for 5-year follow up.

74. The apparatus, tangible medium, computer chip or method as in any one of the preceding embodiments, wherein evaluating the hazard ratio comprises evaluating an age-dependent hazard ratio component and an age-independent hazard ratio component of a hazard ratio of the subject and optionally wherein evaluating the age-independent hazard ratio component comprises evaluating an age-detrended hazard ratio of the subject.

75. The apparatus, tangible medium, computer chip or method as in any one of the preceding embodiments, wherein evaluating the hazard rate comprises evaluating an age-dependent hazard rate component and an age-independent hazard rate component of a hazard rate of the subject and optionally wherein evaluating the age-independent hazard rate component comprises evaluating an age-detrended hazard rate of the subject.

76. The apparatus, tangible medium, computer chip or method as in any one of the preceding embodiments, wherein evaluating the hazard ratio of the subject is performed according to a Cox proportional hazards model.

77. The apparatus, tangible medium, computer chip or method as in any one of the preceding embodiments, wherein evaluating the hazard rate or hazard ratio of the subject is performed according to an accelerated failure time model.

78. The apparatus, tangible medium, computer chip or method as in any one of the preceding embodiments, wherein evaluating the hazard rate or hazard ratio of the subject is performed according to optimization parameters of a Gompertz-Makeham law of mortality.

79. The apparatus, tangible medium, computer chip or method, as in any one of the preceding embodiments, wherein the derived parameter is selected from the group comprising signal, information, action or other object evaluated or created or changed or used or transmitted or indexed or delivered in response to evaluated wellness parameter or in response to change in evaluated wellness parameter of the subject.

80. The apparatus, tangible medium, computer chip or method as in any one of the preceding embodiments, wherein the derived parameter is selected from the group consisting of a frailty index, a physiological resilience, a survival function, a force of mortality, a life expectancy, a life expectancy from birth, and a remaining life expectancy, life span, average life span, maximum life span, healthy life span, health span, fertile life span, age when menopause occurs of the subject, wherein the said derived parameter is evaluated in response to an evaluated hazard rate or hazard ratio of the subject.

81. The apparatus, tangible medium, computer chip or method as in any one of the preceding embodiments, wherein outputting of evaluated wellness parameter is made in the form of adjustment coefficient, or a customized information, content, setting, set of options, service, recommendation, price, term, product or in the form of generation or providing or using or indexation or changing of anything selected from the group: information or object or process, or in the form of triggering or stopping a process.

82. The apparatus, tangible medium, computer chip or method as in any one of the preceding embodiments, further comprising evaluating a status selected from the group consisting of a type 2 diabetes status and a smoking status of the subject in response to the evaluated hazard rate or hazard ratio of the subject.

83. The apparatus, tangible medium, computer chip or method of any one of the preceding embodiments, wherein the evaluated life expectancy, life span, average life span, maximum life span, healthy life span, health span, fertile life span, age when menopause occurs of the subject is classified among a plurality of classes, the plurality of classes selected from the group consisting of short, normal, long and alike.

84. The apparatus, tangible medium, computer chip or method of any one of the preceding embodiments, wherein the evaluated hazard rate or hazard ratio of the subject is classified among a plurality of classes, the plurality of classes selected from the group consisting of low, neutral, high and alike.

85. The apparatus, tangible medium, computer chip or method of any one of the preceding embodiments, wherein the evaluated body mass index of the subject is classified among a plurality of classes, the plurality of classes selected from the group consisting of slim, normal, overweight, and alike.

86. The apparatus, tangible medium, computer chip or method of any one of the preceding embodiments, further comprising evaluating a pregnancy status in response to changes in the body mass index of the subject and optionally wherein the change of the body mass index of the subject comprises a change from a first body mass index to a second body mass index greater than the first body mass index.

87. The apparatus, tangible medium, computer chip or method, as in any one of the preceding embodiments wherein the wellness parameter is evaluated exclusively in response to a combination selected from the group consisting of an input gender of the subject, the feature and the plurality of features extracted from the plurality of measurements obtained by the sensor coupled to the subject, and optionally wherein the wellness parameter is evaluated exclusively in response to a combination selected from the group consisting of feature and the plurality of features extracted from the plurality of measurements obtained by sensor coupled to the subject.

88. The apparatus, tangible medium, computer chip or method of any one of the preceding embodiments, wherein the sensor comprises a sensor externally coupled to the subject.

89. The apparatus, tangible medium, computer chip or method of any one of the preceding embodiments, wherein the sensor comprises a non-invasive sensor.

90. The apparatus, tangible medium, computer chip or method of any one of the preceding embodiments, wherein the sensor is selected from the group consisting of an accelerometer, a MEMS sensor, a MEMS accelerometer, a MEMS gyroscope, a MEMS magnetometer, a pedometer, an optical heart rate monitor and a pulse oximeter sensor.

91. The apparatus, tangible medium, computer chip or method, as in any one of the preceding embodiments, wherein sensor does not have electrodes contacting the subject.

92. The apparatus, tangible medium, computer chip or method, as in any one of the preceding embodiments, wherein sensor comprises a measuring device, wherein the measuring device measures a physical quantity related to physical activity of the subject.

93. The apparatus, tangible medium, computer chip or method, as in any one the preceding embodiments, wherein the measuring device comprises a MEMS sensor and optionally wherein the measuring device comprises a plurality of measuring devices.

94. The apparatus, tangible medium, computer chip or method, as in any one of preceding embodiments, wherein sensor comprises a measuring device selected from the group consisting of an accelerometer, a gyroscope, a magnetometer, and a high-precision location sensor or similar sensor, wherein the sensor is capable of detecting or measuring an aspect of a physical movements of the subject.

95. The apparatus, tangible medium, computer chip or method, as in the preceding embodiment, wherein the measuring device comprises an accelerometer, and optionally wherein the measuring device comprises of a plurality of measuring devices each of the plurality of measuring devices comprises an accelerometer.

96. The apparatus, tangible medium, computer chip or method, as in any one of preceding embodiments, wherein sensor comprises a measuring device, which measures a physical quantity related to a physical activity of the subject and wherein the plurality of measurements comprises readings from said measuring device.

97. The apparatus, tangible medium, computer chip or method, as in any one of preceding embodiments, wherein sensor comprises a set of one or more of measuring devices which measure a physical quantity related to physical activity of the subject; the sensor further comprises a processor which transforms the readings from the set of measuring devices; and wherein the plurality of measurements comprises the transformed readings.

98. The apparatus, tangible medium, computer chip or method, as in the preceding embodiment, wherein the transformations, performed by the processor within the sensor, comprise a combination of noise reduction, normalization according to the measuring device calibration, transformation of the raw measurements into physical activity level, detection of the patterns of motion, combining the measurements from several measuring devices into a single quantity and alike.

99. The apparatus, tangible medium, computer chip or method, as in any one of preceding embodiments, wherein sensor comprises a pedometer, a pulsometer, a pulse oximetry sensor or a similar device capable of reporting level of physical activity of the subject or a quantity related to it.

100. The apparatus, tangible medium, computer chip or method, as in any one of preceding embodiments, wherein sensor is embedded into a single physical embodiment designed to be carried by the subject or to be worn by the subject or to be otherwise attached to the subject.

101. The apparatus, tangible medium, computer chip or method, as in any one of preceding embodiments, wherein sensor comprises a sensor of a mobile device.

102. The apparatus, tangible medium, computer chip or method, as in any one of preceding embodiments, wherein the computer chip comprises one or more of measuring devices comprising a sensor.

103. The apparatus, tangible medium, computer chip or method, as in any one of preceding embodiments, wherein the computer chip comprises the sensor.

104. The apparatus, tangible medium, computer chip or method, as in any one of preceding embodiments, wherein the length, the width and the height of the computer chip are within a range from about 1 mm to about 5 mm, about 1 mm to about 5 mm and about 0.5 mm to about 3 mm, respectively and optionally wherein the length, the width and the height are within a range from about 0.5 mm to about 2.5 mm, about 0.5 mm to about 0.5 mm and about 0.25 mm to about 1.5 mm, respectively.

105. The apparatus, tangible medium, computer chip or method, as in any one of preceding embodiments, wherein the chip comprises an application specific integrated circuit (ASIC).

106. The apparatus, tangible medium, computer chip or method, as in any one of preceding embodiments, wherein the activity sensor comprises an accelerometer.

107. The apparatus, tangible medium, computer chip or method, as in any one of preceding embodiments, wherein the dimensions of the chip comprise a packaging of the chip.

108. The apparatus, tangible medium, computer chip or method of any one of the preceding embodiments, wherein the plurality of measurements received from the sensor is transmitted to a remote server and optionally wherein the plurality of measurements received from sensor is transmitted to a database of the remote server and optionally wherein the plurality of measurements received from sensor is transmitted over the Internet.

109. The apparatus, tangible medium, computer chip or method as in any one of the preceding embodiments, wherein the wellness parameter is evaluated by a processor selected from the group consisting of a remote server and a mobile device configured to be carried by the subject.

110. The apparatus, tangible medium, computer chip or method of any one of the preceding embodiments, wherein the wellness parameter is provided as an output to a user from an output device in proximity to the user, the user selected from the group consisting of the subject and a user who is not the subject.

111. The apparatus, tangible medium, computer chip or method of any one of the preceding embodiments, wherein the output is transmitted to the output device from a remote server and optionally wherein the output is transmitted to the output device from a database of the remote server and optionally wherein the output is transmitted to the output device over an Internet.

112. The apparatus, tangible medium, computer chip or method of any one of the preceding embodiments, wherein the plurality of measurements from the sensor is transmitted to the remote server over the Internet.

113. The apparatus, tangible medium, computer chip or method of any one of the preceding embodiments, wherein the output is selected from the group consisting of a visual display, a message, a message in a social network, a message from a chat-bot, a voice, a sound, a haptic device, a brain-computer interface, and a vibration.

114. The apparatus, tangible medium, computer chip or method of any one of the preceding embodiments, wherein sensor comprises a sensor of a mobile device carried by the subject and wherein the user is not the subject.

115. The apparatus, tangible medium, computer chip or method of any one of the preceding embodiments, wherein the output device is selected from the group consisting of a mobile device carried by the subject, a mobile device worn by the subject, a computer display of a smartphone carried by the subject, a wrist worn device carried by the subject, smart glasses carried by the subject, a smartwatch carried by the subject, and a wristband carried by the subject.

116. The apparatus, tangible medium, computer chip or method of any one of the preceding embodiments, wherein the mobile device comprises a processor configured with instructions to receive the plurality of measurements from the sensor and to transmit the output to the output device of the mobile device.

117. The apparatus, tangible medium, computer chip or method of any one of the preceding embodiments, further comprising a second mobile device configured to be carried by the subject, the second mobile device comprising the sensor.

118. The apparatus, tangible medium, computer chip or method of any one of the preceding embodiments, wherein the sensor comprises a sensor of a smartphone carried by the subject, the smartphone comprising the output device.

119. The apparatus, tangible medium, computer chip or method of any one of the preceding embodiments, wherein the freely moving physical activity of the subject is a physical activity of a free living subject.

120. The apparatus, tangible medium, computer chip or method of any one of the preceding embodiments, wherein subject is selected from the group consisting of a human, a pet, a farm animal, and a laboratory animal.

121. The apparatus, tangible medium, computer chip or method as in any one of the preceding embodiments, wherein the plurality of measurements from the sensor comprises measurement from an independent self-supported movement of the subject, and optionally wherein the independent self-supported movement is selected from the group consisting of crawling, walking, jogging, a movement of the subject over a distance of at least 3 meters, a movement of the subject over a distance of at least 30 meters, and a movement of the subject over a distance of no more than 30 meters.

122. The apparatus, tangible medium, computer chip or method as in any one of the preceding embodiments, wherein the processor comprises a plurality of processors.

123. A tangible medium as in any one of the preceding embodiments, wherein the tangible medium comprises a non-transitory computer readable medium.

124. The apparatus, tangible medium, computer chip or method, as in any one of preceding embodiments, wherein the evaluation of wellness parameter of the subject is performed in response to the received plurality of measurements and based on instructions and parameters generated using machine learning techniques for determining the wellness indication for the subject, and optionally wherein the feature or the plurality of features are extracted according to the instructions and parameters generated using machine learning techniques.

125. The method of embodiment [0545], wherein the method comprises a method for assessing the health and wellness of the subject and the wellness parameter comprise a wellness indication for the subject, the method comprising:

providing a plurality of automated evaluation pipelines, each automated evaluation pipeline independently performing the steps of:
extracting a set of features associated with the subject from the plurality of measurements; receiving instructions and parameters generated using machine learning techniques for determining the wellness indication for the subject; and
processing the extracted set of features and the received instructions and parameters to evaluate the wellness indication for the subject.

126. The method of embodiment 125, further comprising preprocessing the received measurements by performing a preprocessing operation.

127. The method of embodiment 126, wherein preprocessing the received measurements further comprises:

determining whether the received measurements meet a quality requirement; and filtering the measurements that meet the quality requirement by applying transformation to the measurements that meet the quality requirement.

128. The method of embodiment 126, wherein the preprocessing operation is selected from the group consisting of down-sampling a series of measurements to a lower frequency, calculating a magnitude of acceleration, splitting a series of measurements into slices of fixed or variable duration, filtering out slices of measurements with near-zero activity and a plurality of preprocessing operations.

129. The method of embodiment 128, wherein the preprocessing operation comprises a slicing operation that converts the received measurements into a set of slices of predefined length along a time axis to reduce computational costs associated with further processing.

130. The method of embodiment 127, wherein the sensor is selected from the group consisting of an accelerometer and a gyroscope and filtering the measurements comprises converting measurements of time evolution of acceleration along individual axes into measurements of time evolution, absolute value of acceleration, rotational speed, or rotational acceleration.

131. The method of embodiment 127, wherein filtering the measurements further comprises removing artifacts or outliers from the measurements and wherein the transformation is selected from the group consisting of threshold cutoff clipping, frequency band filtering, averaging or smoothing using a moving window, and logarithm scaling.

132. The method of embodiment 125, wherein each of the extracted features has the same predefined number of feature values.

133. The method of embodiment 125, wherein extracting a set of features from the measurements further comprises:

combining the extracted features into a single set of feature values; and
performing an operation to the single set of feature values to yield a final combined feature vector.

134. The method of embodiment 133, wherein the received measurements comprise high and low-frequency representations and wherein combining the extracted features comprises combining features of high and low-frequency representations into a single set of feature values to yield a final combined feature vector.

135. The method of embodiment 125, wherein extracting a set of features from the measurements further comprises:

quantifying the received measurements into bins of different activity levels of activity; analyzing the binned measurements based on determining a statistical distribution among data points of signal levels of the measurements in each bin; and
calculating a transition rate between the signal levels of the measurements in each bin to yield an activity transition matrix as a feature of the received measurements.

136. The method of embodiment 125, further comprising post-processing the extracted set of features by performing a post-processing operation.

137. The method of embodiment 136, wherein the operation is selected from the group consisting of imputation of missing values, logarithm scaling, and dimensionality reduction.

138. The method of embodiment 137, wherein dimensionality reduction further comprises linear detrending or principal component analysis decomposition.

139. The method of embodiment 125, wherein the instructions and parameters for determining a wellness indication for the subject are generated by training and validating a neural network using annotated measurements of freely moving physical activity of a plurality of subjects, each of the subjects having a known wellness indication.

140. The apparatus, tangible medium, computer chip or method of any one of the preceding embodiments, wherein the received measurements comprise high and low-frequency representations and wherein combining the extracted features comprises combining features of high- and low-resolution representations into a single set of features.

141. The apparatus, tangible medium, computer chip or method of any one of the preceding embodiments, wherein the instructions and parameters for extracting evaluating wellness parameter for the subject are generated by machine learning techniques using training and validation set of annotated measurements of freely moving physical activity of a plurality of subjects, each of the subjects having a known wellness indication.

142. The apparatus, tangible medium, computer chip or method of embodiment of anyone of the preceding embodiments, wherein in the method comprises a method of determining a health status of the subject, the wellness parameter corresponding to the health status, and wherein the health status is selected from the group consisting of the age, the hazard rate, the hazard ratio, the type 2 diabetes status and the body mass index.

We claim:

1. A method to evaluate a wellness parameter or a derived parameter of a subject in response to freely moving physical activity of the subject, the method comprising:
evaluating the wellness parameter or the derived parameter with a model in response to the plurality of features, extracted from the plurality of measurements of freely moving physical activity of the subject received by a sensor externally coupled to the subject, wherein the wellness parameter is selected from the group consisting of an age, a hazard rate, a hazard ratio, a type 2 diabetes status and a body mass index, and wherein the derived parameter is evaluated in response to the evaluated wellness parameter and optionally wherein the derived parameter is evaluated in response to a plurality of evaluated wellness parameters.

2. The method of claim 1, wherein the feature is derived from activity sensor data having frequencies within a range selected from the group consisting of any two of the following values: 0.1 Hz, 0.01 Hz, 0.001 Hz, 0.0001 Hz, 0.0002 Hz, 0.000011 Hz, 0.00001 Hz and 0.000001 Hz.

3. The method of claim 1, wherein the plurality of measurements comprises a low-resolution series of measurements, the series of measurements comprising an interval between successive measurements of the series, wherein a physical activity level of the subject corresponds to a level of overall physical activity of the subject over a period of time, and wherein the period of time corresponding to the level of overall physical activity is not less than one tenth of the interval between successive measurements, not less than 5 s, and is not longer than ten times the length of the interval between measurements and is not longer than 1 hour.

4. The method of claim 1, wherein feature or the plurality of features is selected from the group consisting of autocorrelation, power spectral density and transition rates between different activity states and probability distribution properties.

5. The method of claim 4, wherein the transition rates between different activity states comprise a set of transition rates between activity states, and optionally wherein transition rates between different activity states comprise a matrix of transition rates between said activity states and optionally wherein said transition rates between different activity states comprise a full set of transition rates between all activity states and optionally wherein said transition rates between different activity states comprise the matrix of transition rates between all activity states.

6. The method of claim 1, further comprising outputting wellness or derived parameter, wherein such parameter is evaluated with a combined set of features comprising data from a transition matrix and a power spectral density from the plurality of measurements.

7. The method of claim 1, further comprising step of extracting a feature from the plurality of measurements prior to evaluating of the wellness parameter and
step of post-processing the extracted set of features by performing a post-processing procedure, wherein the post-processing procedure is selected from the group consisting of imputation of missing or near-zero values, logarithm scaling, and dimensionality reduction.

8. The method of claim 7, wherein dimensionality reduction further comprises linear detrending or principal component analysis decomposition.

9. The method of claim 1, wherein the wellness parameter comprises a plurality of wellness parameters of the subject, the plurality of wellness parameters selected from the group consisting of the age, the hazard rate, the hazard ratio, the type 2 diabetes status and the body mass index and wherein the plurality of wellness parameters comprises a first wellness parameter and a second wellness parameter and wherein the second wellness parameter is evaluated in response to a combination of the first evaluated wellness parameter and the feature or the plurality of features.

10. The method of claim 1, wherein the wellness parameter comprises the diabetes type 2 status and wherein an accuracy of the evaluated diabetes type 2 status corresponds to a sensitivity and a selectivity selected from the group consisting of a sensitivity of at least 0.6 and at a selectivity of least 0.8, a sensitivity within a range from about 0.6 to about 0.9 and a selectivity within a range from about 0.8 to about 0.95, a sensitivity of at least 0.75 and a selectivity of at least 0.75, and a sensitivity within a range from about 0.75 to about 0.95 and a selectivity within a range from about 0.75 to about 0.95 and optionally wherein the accuracy is determined for a group of subjects and optionally wherein the subject is a member of the group of subjects and optionally wherein the subject is not a member of the group of subjects.

11. The method of claim 1, further comprising evaluating hazard rate of a subject with the model in response to the evaluated hazard ratio of the subject combined with a reference hazard rate and optionally wherein the reference hazard rate comprises an average hazard rate of a reference population.

12. The method of claim 1, further comprising evaluating hazard ratio of a subject with the model in response to the evaluated hazard rate of the subject combined with a reference hazard rate and optionally wherein the reference hazard rate comprises an average hazard rate of a reference population.

13. The method of claim 1, wherein an accuracy of the evaluated hazard rate or hazard ratio is greater than an area under a receiver operating curve (ROC AUC) of about 0.6 and optionally wherein the ROC AUC is within a range from about 0.6 to about 0.9 and optionally wherein the accuracy is determined for a group of subjects for which the ROC AUC is determined and optionally wherein the subject is a member of the group of subjects and optionally wherein the subject is not a member of the group of subjects.

14. The method of claim 1, wherein evaluating the hazard ratio comprises evaluating an age-dependent hazard ratio component and an age-independent hazard ratio component of a hazard ratio of the subject and optionally wherein evaluating the age-independent hazard ratio component comprises evaluating an age-detrended hazard ratio of the subject.

15. The method of claim 1, wherein evaluating the hazard rate comprises evaluating an age-dependent hazard rate component and an age-independent hazard rate component of a hazard rate of the subject and optionally wherein evaluating the age-independent hazard rate component comprises evaluating an age-detrended hazard rate of the subject.

16. The method of claim 1, wherein evaluating the hazard ratio of the subject is performed according to a Cox proportional hazards model.

17. The method of claim 1, wherein evaluating the hazard rate or hazard ratio of the subject is performed according to an accelerated failure time model.

18. The method of claim 1, wherein evaluating the hazard rate or hazard ratio of the subject is performed according to optimization parameters of a Gompertz-Makeham law of mortality.

19. The method of claim 1, wherein the derived parameter is selected from the group comprising signal, information, action or other object evaluated or created or changed or used or transmitted or indexed or delivered in response to evaluated wellness parameter or in response to change in evaluated wellness parameter of the subject.

20. The method of claim 1, wherein the derived parameter is selected from the group consisting of a frailty index, a physiological resilience, a survival function, a force of mortality, a life expectancy, a life expectancy from birth, and a remaining life expectancy of the subject, life span, average life span, maximum life span, healthy life span, health span, fertile life span, age when menopause occurs, wherein the said derived parameter is evaluated in response to an evaluated hazard rate or hazard ratio of the subject.

21. The method of claim 1, further comprising an outputting of evaluated wellness parameter, wherein such outputting is made in the form of adjustment coefficient, or a customized information, content, setting, set of options, service, recommendation, price, term, product or in the form of generation or providing or using or indexation or changing of anything selected from the group: information or object or process, or in the form of triggering or stopping a process.

22. The method of claim 1, further comprising evaluating a status selected from the group consisting of a type 2 diabetes status and a smoking status of the subject in response to the evaluated hazard rate or hazard ratio of the subject.

23. The method of claim 1, wherein the wellness parameter is evaluated exclusively in response to a combination selected from the group consisting of an input gender of the subject, the feature and the plurality of features extracted from the plurality of measurements obtained by the sensor coupled to the subject, and optionally wherein the wellness parameter is evaluated exclusively in response to a combination selected from the group consisting of feature and the plurality of features extracted from the plurality of measurements obtained by sensor coupled to the subject.

24. The method of claim 1, wherein sensor comprises a measuring device, wherein the measuring device measures a physical quantity related to physical activity of the subject, wherein the measuring device comprises an accelerometer, and optionally wherein the measuring device comprises of a plurality of measuring devices each of the plurality of measuring devices comprises an accelerometer.

25. The method of claim 1, wherein the plurality of measurements received from the sensor is transmitted to a remote server and optionally wherein the plurality of measurements received from sensor is transmitted to a database of the remote server and optionally wherein the plurality of measurements received from sensor is transmitted over the Internet.

26. The method of claim 1, wherein the freely moving physical activity of the subject is a physical activity of a free living subject.

27. The method of claim 1, wherein the evaluation of wellness parameter of the subject is performed in response to the received plurality of measurements and based on instructions and parameters generated using machine learning techniques for determining the wellness indication for the subject, and optionally wherein the feature or the plurality of features are extracted according to the instructions and parameters generated using machine learning techniques.

28. The method of claim 27, wherein the instructions and parameters for determining a wellness indication for the subject are generated by training and validating a neural network using annotated measurements of freely moving physical activity of a plurality of subjects, each of the subjects having a known wellness indication.

29. A non-transitory readable medium comprising computer-executable instructions stored thereon, wherein the computer-executable instructions instruct one or more processors to perform a method comprising:
evaluating the wellness parameter or the derived parameter with a model in response to the plurality of features, extracted from the plurality of measurements of freely moving physical activity of the subject received by a sensor externally coupled to the subject, wherein the wellness parameter is selected from the group consisting of an age, a hazard rate, a hazard ratio, a type 2 diabetes status and a body mass index, and wherein the derived parameter is evaluated in response to the evaluated wellness parameter and optionally wherein the derived parameter is evaluated in response to a plurality of evaluated wellness parameters.
